SEQUENCE LISTING (17 sequences)

SEQ ID NO: 1 - DNA, 6083 bp, synthetic construct
  misc_feature 1..6083: note = Description of sequence: Plasmid, pSens4NB2
  source 1..6083: mol_type = other DNA
  variation 3629..3630: note = n is a, c, g or t
tggttcgttgtgatggcggtaggaatgtaatcgttaatccgcaaataacgtaaaaacccgcttcggcgggtttttttatggggggagtttagggaaagagcatttgtcatcccgttgaatatggctcgcatcttatcgagcatactatcacgtcggcgaccactagtcagttaacgcaagggcatgggctgtcgacctttgaaaagtaccttgacggcgtatctttgctttctataatgagtgcttactcactcatacaatagtcagtcataagtctgggctaagcccactgatgagtcgctgaaatgcgacgaaacttatgacctctacaaataattttgtttaacgtaaacctccgggttaataaggagtaattatggcatccaagggcgaggagctctttactggcgtagtaccaattctcgtagagctcgatggcgatgtaaatggccataagttttccgtacgcggcgagggcgagggcgatgcaactaacggcaagctcactctcaagtttatttgtactactggcaagctcccagtaccatggccaactctcgtaactactctgacctatggcgtacaatgtttttcccgctatccagatcacatgaagcaacatgatttttttaagtccgcaatgccagagggctatgtacaagagcgcactattagctttaaggatgatggcacctataagactcgcgcagaggtaaagtttgagggcgatactctcgtaaatcgcattgagctcaagggcattgattttaaggaggatggcaatattctcggccataagctggagtataatttcaattcccataatgtatatattaccgcagataagcaaaagaatggcattaaggcgaattttaagattcgccataatgtggaggatggctccgtacaactcgcagatcattatcaacaaaatactccaattggcgatggcccagtactcctcccagataatcattatctctccactcaatccgtgctctccaaagatccaaatgagaagcgcgatcacatggtactcctggagtttgtaactgcagcaggcattactcatggcatggatgagctctataagctcgagcaccaccaccaccaccactgataatccaaacctgttatatgttagctgagactagttggaagtgtggctgtcctcaagcgttttagttcgtcggtcagtttcacctgatttacgtaaaaacccgcttcggcgggtttttgcttttggaggggcagaaagatgaatgactgtcggccattcgatggtgtcgggtagcataaccccttgtgatacctttgccatgtttcagaaacaactctggcgcatcgggcttggaccaaaacgaaaaaaggccgctttcgcggcctcttttctggaatttggtaccgagctcacagcggcgatagtcagatagctagaccgtatgttaccgagcctgcactcctcgaattcttaggattattactgctcttcgcgcgtaagtgcgcgccacatagcctcgaagcccaacgcaatgtactcaccagcgcgagccgggtcgcgcgcagcgaaatccatagtcgtctcagcaagcgccaagaacaacccgtcgccgaaggcgcggtactcgtcggacataaacaccataagaacgccacggtggtcgaggtcgcgtaactccgggaacatatcatccgcgcgttgttcggtttccttcgtcaacttttcagaaaccgccaactgacgaatggcacgatggcgagctgggtggttcaatccccagctaatataactgttccagataaaacgggtcatcatcttagcgtcagtaatagaacgatccaattccatgatcattgattggcacatgtcctgggtcaaatgtaagtaaagggtgttgatcaactcatctttcgttgcgaaatagcggaacaacgtcccttccgcaactcccgcattgcgtgcaattacagcggtactagcggcaatgcctgattgcgcgatggcttgagttgccgcttcaagcaatgcctgctttttgtcctcagactttgggcgagcaaccatatactaacctccttctgatacgtggttccgttaaacaaaattatttgtagaggccccatttcgtccttttggactcatcaggggtggtacacaccaccctatggggctcgtaattgctagcataatccctaggactgagctagctatcagggtacttttcaaaggtcgacagcccatgcccttgcgttcggcaggtgtacaatgatacgaggtaatgaagatgaagtccatacaatcgatagattgggaccaaaacgaaaaaaggggagcggtttcccgctcccctcttttctggaatttggtaccgagtcgcacctgattgcccgacattatcgcacggtgtctcatctctgataacgcatattgtcgttagaactcggcgcggccgctcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcattttagcttccttagctcctgaaaatctcgataactcaaaaaatacgcccggtagtgatcttatttcattatggtgaaagttggaacctcttacgtgccgatcacgtctcattttcgccaaagttggccagggcttcccggtatcaacagggacaccaggatttatttatnntgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccatcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgacgcggccgcggcatcagagcagattgtactgtgtcctcaa

SEQ ID NO: 2 - DNA, 3987 bp, synthetic construct
  misc_feature 1..3987: note = Description of sequence: Plasmid, Nb4OMT-A53M
  source 1..3987: mol_type = other DNA
atcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccgcagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaaggccctctccaagaccgagccatcaacaaagcgtctcgctgaggtttcatggagcctctggttcatctccggcaattaaaaaagcggctaaccacgccgctttttttacgtctgcaggaacgggctgtcgacctttgaaaagttcgtttaccgctagctcagtcctaggtacaattacagccatcgtacgagccctggctgagcacagctgtcaccggatgtgctttccggtctgatgagtccgtgaggacgaaacagcctctacaaataattttgtttaaactagtgaaccacgaggcctacatatgggtgcttcaattgatgactactccttggtacataaaaacatcttgcattccgaagatctgctgaaatacattcttgaaaccagtgcatatcctcgcgaacacgaacaattaaagggtctgcgtgaggttactgaaaagcacgaatggtcatccatgttagtaccggcagacgaaggtttattcctgtcaatgttactgaagttgatgaatgcaaaacgtactatcgaaatcggcgtgtacacgggatacagcctgttaacaacggcattggctttaccggaggatggtaaaattacggcgatcgatgtaaataagagttattacgaaatcggattgccctttattcagaaggcgggcgtggaacacaagatcaactttattgagtcggaagcgcttcccgtgctggatcaaatgttagaggagatgaaggaggaggatttatacgactacgcttttgtggatgctgataaaagcaattatgccaattaccatgagcgtcttgtaaaacttgtacgtatcggtggtgccatcctgtacgacaatacactgtggtatggttctgttgcgtacccggaataccccggtctgcatccagaggaagaagtcgcgcgtctgagctttcgtaacttgaatacctttttagcagcagatcctcgcgtagaaattagtcaagtctcaattggtgatggcgtgaccatttgtcgccgcttgtattaataatcctatcgccactttcagccaaaaaacttaagaccgccggtcttgtccactaccttgcagtaatgcggtggacaggatcggcggttttcttttctcttctcaacacccttcgcgtcaacacttttccgccaaggagacggttggtcaggttttcgggaggtgtggctggaagttcctatactttctagagaataggaacttctttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatcgcgcggtatcattgcagcactggggccagatggtaagccctcccgtatcgtagttatctacacgacggggagtcaggcaactatggatgaacgaaatagacagatcgctgagataggtgcctcactgattaagcattggtaagttgtgatggcggtaggaatgtaatcgttaatccgcaaataacgtaaaaacccgcttcggcgggtttttttatggggggagtttagggaaagagcatttgtcatcccgttgaatatggctcccttaacgtgaggaagttcctatactttctagagaataggaacttctacagatggacttgggttggcggtttcaggagtctgcaaaacgtctgcgacctgagcaacaacatgaatggtcatcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaa

SEQ ID NO: 3 - protein, 239 aa, synthetic construct
  REGION 1..239: note = Description of sequence: Natural enzyme, norbelladine 4'-O-Methyltransferase
MGASIDDYSLVHKNILHSEDLLKYILETSAYPREHEQLKGLREVTEKHEWSSALVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 4 - protein, 239 aa, synthetic construct
  REGION 1..239: note = Description of sequence: Improved enzyme variant A53M
MGASIDDYSLVHKNILHSEDLLKYILETSAYPREHEQLKGLREVTEKHEWSSMLVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 5 - protein, 239 aa, synthetic construct
  REGION 1..239: note = Description of sequence: Improved enzyme variant S159E
MGASIDDYSLVHKNILHSEDLLKYILETSAYPREHEQLKGLREVTEKHEWSSALVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKENYANYHERLVKLVRIGGAILYDNTLWYGSVAYPEYPGLHPEEEVARLSFRNLNTFLAADPRVEISQVSIGDGVTICRRLY

SEQ ID NO: 6 - protein, 239 aa, synthetic construct
  REGION 1..239: note = Description of sequence: Improved enzyme variant V203E
MGASIDDYSLVHKNILHSEDLLKYILETSAYPREHEQLKGLREVTEKHEWSSALVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKSNYANYHERLVKLVRIGGAILYDNTLWYGSVAYPEYPGLHPEEEEARLSFRNLNTFLAADPRVEISQVSIGDGVTICRRLY

SEQ ID NO: 7 - protein, 239 aa, synthetic construct
  REGION 1..239: note = Description of sequence: Improved enzyme variant H17K
MGASIDDYSLVHKNILKSEDLLKYILETSAYPREHEQLKGLREVTEKHEWSSALVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 8 - protein, 239 aa, synthetic construct
  REGION 1..239: note = Description of sequence: Improved enzyme variant E36P-G40E
MGASIDDYSLVHKNILHSEDLLKYILETSAYPREHPQLKELREVTEKHEWSSALVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 9 - protein, 194 aa, synthetic construct
  REGION 1..194: note = Description of sequence: Natural biosensor
MVARPKSEDKKQALLEAATQAIAQSGIAASTAVIARNAGVAEGTLFRYFATKDELINTLYLHLKQDLCQSMIMELDRSITDAKMMTRFIWNSYISWGLNHPARHRAIRQLAVSEKLTKETEQRADDMFPELRDLCHRSVLMVFMSDEYRAFGDGLFLALAETTMDFAARDPARAGEYIALGFEAMWRALTREEQ

SEQ ID NO: 10 - protein, 194 aa, synthetic construct
  REGION 1..194: note = Description of sequence: Improved biosensor variant L133T, C134E, S127T
MVARPKSEDKKQALLEAATQAIAQSGIAASTAVIARNAGVAEGTLFRYFATKDELINTLYLHLKQDLCQSMIMELDRSITDAKMMTRFIWNSYISWGLNHPARHRAIRQLAVSEKLTKETEQRADDMFPELRDTEHRTVLMVFMSDEYRAFGDGLFLALAETTMDFAARDPARAGEYIALGFEAMWRALTREEQ

SEQ ID NO: 11 - protein, 194 aa, synthetic construct
  REGION 1..194: note = Description of sequence: Improved biosensor variant K63T, L66M
MVARPKSEDKKQALLEAATQAIAQSGIAASTAVIARNAGVAEGTLFRYFATKDELINTLYLHLTQDMCQSMIMELDRSITDAKMMTRFIWNSYISWGLNHPARHRAIRQLAVSEKLTKETEQRADDMFPELRDLCHRSVLMVFMSDEYRAFGDGLFLALAETTMDFAARDPARAGEYIALGFEAMWRALTREEQ

SEQ ID NO: 12 - protein, 194 aa, synthetic construct
  REGION 1..194: note = Description of sequence: Improved biosensor variant K63R, M70T
MVARPKSEDKKQALLEAATQAIAQSGIAASTAVIARNAGVAEGTLFRYFATKDELINTLYLHLRQDLCQSTIMELDRSITDAKMMTRFIWNSYISWGLNHPARHRAIRQLAVSEKLTKETEQRADDMFPELRDLCHRSVLMVFMSDEYRAFGDGLFLALAETTMDFAARDPARAGEYIALGFEAMWRALTREEQ

SEQ ID NO: 13 - protein, 194 aa, synthetic construct
  REGION 1..194: note = Description of sequence: Improved biosensor variant K63T, L66M, C134D, S137G
MVARPKSEDKKQALLEAATQAIAQSGIAASTAVIARNAGVAEGTLFRYFATKDELINTLYLHLTQDMCQSMIMELDRSITDAKMMTRFIWNSYISWGLNHPARHRAIRQLAVSEKLTKETEQRADDMFPELRDLDHRGVLMVFMSDEYRAFGDGLFLALAETTMDFAARDPARAGEYIALGFEAMWRALTREEQ

SEQ ID NO: 14 - protein, 194 aa, synthetic construct
  REGION 1..194: note = Description of sequence: Improved biosensor variant K63T, L66M, C134D, S137N
MVARPKSEDKKQALLEAATQAIAQSGIAASTAVIARNAGVAEGTLFRYFATKDELINTLYLHLTQDMCQSMIMELDRSITDAKMMTRFIWNSYISWGLNHPARHRAIRQLAVSEKLTKETEQRADDMFPELRDLDHRNVLMVFMSDEYRAFGDGLFLALAETTMDFAARDPARAGEYIALGFEAMWRALTREEQ

SEQ ID NO: 15 - protein, 194 aa, synthetic construct
  REGION 1..194: note = Description of sequence: Improved biosensor variant K63T, L66M, C134E, S137D
MVARPKSEDKKQALLEAATQAIAQSGIAASTAVIARNAGVAEGTLFRYFATKDELINTLYLHLTQDMCQSMIMELDRSITDAKMMTRFIWNSYISWGLNHPARHRAIRQLAVSEKLTKETEQRADDMFPELRDLCHRDVLMVFMSDEYRAFGDGLFLALAETTMDFAARDPARAGEYIALGFEAMWRALTREEQ

SEQ ID NO: 16 - protein, 194 aa, synthetic construct
  REGION 1..194: note = Description of sequence: Improved biosensor variant K63T, L66M, C134N, S137G
MVARPKSEDKKQALLEAATQAIAQSGIAASTAVIARNAGVAEGTLFRYFATKDELINTLYLHLTQDMCQSMIMELDRSITDAKMMTRFIWNSYISWGLNHPARHRAIRQLAVSEKLTKETEQRADDMFPELRDLNHRGVLMVFMSDEYRAFGDGLFLALAETTMDFAARDPARAGEYIALGFEAMWRALTREEQ

SEQ ID NO: 17 - protein, 239 aa, synthetic construct
  REGION 1..239: note = Description of sequence: Improved enzyme variant H17R Mutation
MGASIDDYSLVHKNILRSEDLLKYILETSAYPREHEQLKGLREVTEKHEWSSALVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKSNYANYHERLVKLVRIGGAILYDNTLWYGSVAYPEYPGLHPEEEVARLSFRNLNTFLAADPRVEISQVSIGDGVTICRRLY

(19) United States
(12) Patent Application Publication
(10) Pub. No.: US 2024/0336900 A1
(43) Pub. Date: Oct. 10, 2024

(54) METHODS AND COMPOSITIONS RELATED TO MODIFIED METHYLTRANSFERASES AND ENGINEERED BIOSENSORS

(71) Applicant: BOARD OF REGENTS, THE UNIVERSITY OF TEXAS SYSTEM, Austin, TX (US)

(72) Inventors: Andrew Ellington, Austin, TX (US); Simon d’Oelsnitz, Austin, TX (US); Daniel Diaz, Austin, TX (US)

(21) Appl. No.: 18/436,635

(22) Filed: Feb. 8, 2024

Related U.S. Application Data
(60) Provisional application No. 63/493,065, filed on Mar. 30, 2023.

(51) Int. Cl. C12N 9/10; C12P 17/18; G16B 15/00; G16B 40/20

(52) U.S. Cl. CPC C12N 9/1007; C12P 17/18; G16B 15/00; G16B 40/20

(57) ABSTRACT

This invention relates to non-naturally occurring methyltransferase, wherein said methyltransferase can methylate norbelladine to form 4-O'Methylnorbelladine. The invention is also drawn to a biosensor for detecting 4-O'Methylnorbelladine, wherein the biosensor comprises an engineered substrate-promiscuous regulator, wherein said substrate-promiscuous regulator has been engineered to interact more efficiently with 4-O'Methylnorbelladine than does a naturally occurring substrate promiscuous regulator; and further wherein the biosensor is engineered to provide an output signal, wherein said output signal is generated when the biosensor interacts with 4-O'Methylnorbelladine.

### CROSS-REFERENCE TO RELATED APPLICATION

This application claims benefit of U.S. Provisional Application No. 63/493,065, filed Mar. 30, 2023, incorporated herein by reference in its entirety.

### GOVERNMENT SUPPORT CLAUSE

This invention was made with government support under Grant no. R01 EB026533 awarded by the National Institutes of Health, Grant no. 70NANB21H100 awarded by the National Institute of Standards and Technology and Grant no. FA9550-14-1-0089 awarded by the Air Force Office of Scientific Research. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Jan. 31, 2024, is entitled “10046-517US1_ST26.xml”, and is 35,922 bytes in size.

### BACKGROUND

Alkaloids produced by the Amaryllidoideae subfamily of flowering plants have great therapeutic promise, including anticancer, fungicidal, antiviral, and acetylcholinesterase inhibition properties. Among the approximate ˜600 reported AAs, those derived from the lycorine, haemanthamine, and narciclasine scaffolds have been used as lead molecules in anticancer research (Berkov, 2020; Evidente, 2009; Cahlikova, 2021; Roy; 2018). One of the most notable Amaryllidoideae alkaloids (AAs) is galantamine, a selective and reversible acetylcholinesterase inhibitor that is a licensed treatment for mild to moderate symptoms of Alzheimer's disease and a promising scaffold for drug design (Bhattacharya, 2015; Mucke, 2015). Due to galantamine's challenging synthesis, global supplies largely rely on isolating the low quantities (0.3% dry weight) that accumulate in harvested daffodils, ultimately resulting in an extremely expensive and environmentally-dependent supply chain (Akram, 2021; Marco-COntelles, 2006). In an effort to improve galantamine production, new agricultural techniques are currently being tested to boost daffodil-sourced yields (Fraser, 2021; Effect of Fertilizers on Galanthamine). The biosynthesis of galantamine is described in Mehta et al. 2023.

A promising alternative to amaryllidaceae alkaloid extraction from plants is microbial fermentation. Recently, long plant pathways have been reconstituted into microbial hosts for the production of therapeutic benzylisoquinoline alkaloids (Thodey, 2014; Payne, 2021), tropane alkaloids (Srinivasan, 2020), and monoterpene indole alkaloids (Zhang, 2022). While the complete biosynthetic pathway for any AA with therapeutic value has not yet been elucidated, recent studies have characterized early pathway enzymes responsible for the biosynthesis of 4′-O-Methyl-Norbelladine, the last common intermediate before AA pathway branches diverge (Kilgore, 2016). Furthermore, semi-synthetic methods have been proposed using characterized enzymes to generate advanced intermediates (Ehrenworth, 2017).

What is needed in the art are both methyl transferases for biosynthesis of 4′-O-Methyl-Norbelladine, as well as high-throughput screens using genetic biosensors (d′Oelsnitz, 2022; Schendzielorz, 2014; Zhang, 2020; Tang, 2013). Further, what is needed is using artificial intelligence to guide protein design (Lu, 2022; Hie, 2022; Greenhalgh, 2021; Wu, 2019), yielding enzymes and pathways with improved stability and activity.

### SUMMARY

Disclosed herein is a non-naturally occurring methyltransferase, wherein said methyltransferase can methylate norbelladine to form 4-O'Methylnorbelladine.

Also disclosed herein is a method of preparing an amaryllidaceae alkaloid, wherein the amaryllidaceae alkaloid composition requires methylation of norbelladine to form 4-O'Methylnorbelladine, the method comprising: (a) culturing a host cell under suitable conditions, wherein the host cell comprises nucleic acid encoding a non-naturally occurring methyltransferase; (b) exposing the methyltransferase to norbelladine; and (c) allowing the methyltransferase to methylate norbelladine, thereby producing a methylated composition of interest.

Also disclosed is a biosensor for detecting 4-O'Methylnorbelladine, wherein the biosensor comprises an engineered substrate-promiscuous regulator, wherein said substrate-promiscuous regulator has been engineered to interact more efficiently with 4-O'Methylnorbelladine than does a naturally occurring substrate promiscuous regulator; and further wherein the biosensor is engineered to provide an output signal, wherein said output signal is generated when the biosensor interacts with 4-O'Methylnorbelladine.

Further disclosed is a kit comprising a 4-O'Methylnorbelladine biosensor comprising an engineered substrate-promiscuous regulator, wherein said substrate-promiscuous regulator has been engineered to interact more efficiently with 4-O'Methylnorbelladine than does the naturally occurring substrate promiscuous regulator.

### DESCRIPTION OF DRAWINGS

FIG. 1A-C shows identifying a biosensor responsive to amaryllidaceae alkaloid intermediates. (a). Abbreviated biosynthetic plant pathways for therapeutic amaryllidaceae alkaloids. (b) Response of the RamR transcription factor to amaryllidaccac alkaloid pathway intermediates norbelladine and 4-Ome-norbelladine. Error bars represent the S.E.M+/− the mean. Experiments were conducted in biological triplicate. (c) Structure of RamR (PDB: 3VVX) docked with 4-OMe-norbelladine using GNINA (see Methods). Predicted ligand-interacting residues are highlighted green.

FIG. 2A-G shows evolving a highly specific biosensor for 4′-O-Methyl-Norbelladine. (a) Schematic illustrating the mutations that resulted after round one (4NB1) and round two (4NB2) of RamR evolution towards 4-OMe-norbelladine. (b) Dose response measurements of WT RamR, 4NB1, and 4NB2 mutants with 4-OMe-norbelladine. (c) Relative response of WT RamR, 4NB1, and 4NB2 mutants to norbelladine and 4-Ome-norbelladine. (d, c) Structure of Alpha Folded 4NB2 docked with 4-OMe-norbelladine. Predicted ligand interactions with WT residues, mutations that arose in 4NB1, and mutations that arose in 4NB2 are colored gray, orange, and green, respectively. (f) Correlation between fluorescent response measured with the 4NB2 sensor and 4-OMe-norbelladine measured with HPLC. (g) The distribution of fluorescent cell populations in response to 4-OMe-norbelladine concentration. All data was performed in biological triplicate. Error bars represent the S.E.M+/− the mean.

FIG. 3A-C shows monitoring 40MT activity with the 4NB2 biosensor. (a) Schematic representation of the biosensor-monitored enzymatic reaction within E. coli cells. Light blue and dark blue hexagons denote norbelladine and 4-OMe-norbelladine, respectively. (b) Correlation between cell population fluorescence and biosynthesized 4-OMe-norbelladine, measured by HPLC. Error bars represent S.E.M. +/− the mean. (c) Chromatographic traces of supernatant collected from E. coli cells expressing an active Nb4OMT enzyme (green) or an empty plasmid (red). Traces for norbelladine and 4-OMe-norbelladine standards are shown in light blue and dark blue, respectively.

FIG. 4A-C shows synergizing AI and biosensors to improve 4NB titers. (a) Fluorescent signal produced from E. coli cells containing the 4-OMe-norbelladine reporter plasmid (pSens-4NB2) and expressing cither an empty plasmid (TAA), the wild-type Nb4OMT enzyme (WT), or AI-designed Nb4OMT mutants, when cultured with 100 uM of norbelladine at 37° C. The purple horizontal line denotes the fluorescent signal produced from culturing the wild-type Nb4OMT enzyme. Error bars represent the S.E.M. +/− the mean (b) Time-dependent fluorescent signals produced by E. coli cells containing the 4-OMe-norbelladine reporter plasmid (pSens-4NB2) and expressing Nb4OMT (WT) or AI-designed mutants. (c) Ion-extracted chromatograms of chemical standards (blue) or the supernatant of cells expressing Nb4OMT or AI-designed mutants (purple) cultured with norbelladine.

FIG. 5 shows structural depiction of RamR library designs. The structure of RamR (PDB: 3VVX) was docked with 4-OMe-norbelladine (purple) using the GNINA1.0 docking software. The side chains of residues targeted for site-saturation mutagenesis are color coded as follows. Orange: K63, L66, M71; Green: E120, A123, D124; Blue: L133, C134, S137.

FIG. 6A-D shows sensitivity and selectivity of RamR mutants evolved for 4-OMe-norbelladine. (a, c) Dose response measurements and genotype of generation one RamR sensors. (b,d) Selectivity

FIG. 7 shows plasmid architecture for the one-plasmid 4-O'Methyl-norbelladine reporter system.

FIG. 8 shows fluorescent response of 4NB2 with varying levels of Nb40MT expression and precursor (norbelladine) supplementation. TAA represents an empty plasmid control in place of the Nb4OMT gene. Measurements were performed in biological triplicate and error bars represent the S.E.M. +/− the mean.

FIG. 9 shows substrate and product of in vivo Nb4OMT reaction measured with HPLC. E. coli cells expressing the wild-type Nb4OMT enzyme were cultured with varying amounts of norbelladine for 18 hours and the concentrations of norbelladine and 4-OMe-norbelladine were subsequently measured. Measurements were performed in biological triplicate and error bars represent the S.E.M. +/− the mean.

FIG. 10A-B shows LC/MS analysis of the Nb4OMT-catalyzed in vivo reaction. E. coli cells expressing wild-type Nb4OMT were cultured for 24 hours with 500 uM of norbelladine and the culture supernatant was filtered and analyzed using LC/MS. (a) Ion-extracted chromatogram of the reaction product. The 274.1438 m/z ratio was used for extraction, since this m/z ratio is expected from all single methylated norbelladine products. (b) Statistics covering relative ion counts for the minor and major products.

FIG. 11A-C shows architecture details of 3DResNet: A) Residual Identity block B) Residual Convolutional block C) Full architecture of 3DResNet describing how a voxelized microenvironment (batch_size, 9 channels, 20x, 20y, 20z) is fed through the 3D residual feature extractor and converted into a 400-dimensional feature vector and then passed into a classifier to generate 20 amino acid probability distribution. Conv 3D: 3D Convolution layer, convolution kernel dimensions: 1×1×1 or 3×3×3, F1 and F2 are the number of feature maps generated by the convolution layer. S=2: stride of 2 used for that convolution layer else S=1 was used, ReLU: Rectified Linear Unit, Batch Norm: 3D Batch Normalization layer. Each convolution layer had L2 weight decay regularization set to 0.001. Batch normalization was instantiated with default hyperparameters.

FIG. 12A-F shows fluorescent response of cells containing single 40MT variants across two precursor supplementation concentrations and three fermentation temperatures. In panels a-c, 100 uM of norbelladine was supplemented in the media, whereas for panels d-f, 1000 uM was supplemented. Measurements were performed in biological triplicate. Error bars represent the S.E.M. +/− the mean.

FIG. 13 shows HPLC-measured substrate and product concentrations resulting from in vivo reactions with Nb4OMT variants. Reactions were carried out within E. coli cells cultured for 24 hours at 37° C. with 500 uM of norbelladine supplemented in the media. The resulting culture supernatant was filtered and compound concentrations were determined using HPLC. Mutations relative to the wild-type Nb4OMT sequence are labeled (for example, “53M”). Measurements were performed in biological triplicate. Error bars represent the S.E.M. +/− the mean.

FIG. 14A-B shows LC/MS analysis of the Nb4OMT variant-catalyzed in vivo reaction. E. coli cells expressing Nb4OMT variants were cultured for 24 hours with 500 uM of norbelladine and the culture supernatant was filtered and analyzed using LC/MS. (a) Ion-extracted chromatogram of the reaction product. The 274.1438 m/z ratio was used for extraction, since this m/z ratio is expected from all single methylated norbelladine products. (b) Statistics covering relative ion counts for the minor and major products. Variant name to mutation mapping is as follows, WT: the natural Nb4OMT sequence; 53: A53M; 36-40: E36P+G40E; 36-40-53: E36P+G40E+A53M.

FIG. 15A-B shows chemical synthesis (A) and NMR validation (B) of norbelladine. NMR spectra were taken on the 500 MHZ Bruker prodigy at University of Texas at Austin. NMR solvents (CD3OD). ¹H NMR (500 MHZ, MeOD) δ 6.87 (d, J=8.2 Hz, 2H), 6.76 (m, J=7.8 Hz, 2H), 6.67 (d, J=8.2 Hz, 2H), 6.63 (d, J=8.1 Hz, 1H), 3.97 (q, J=5.6 Hz, 1H), 3.46 (q, J=7.6 Hz, 1H), 2.87 (m, J=4.6 Hz, 2H), 2.74 (m, J=7.6 Hz, 1H), 2.61 (m, J=3.5 Hz, 1H).

FIG. 16 shows sequences with color-coded annotations of plasmids used. SEQ ID NOS: 1 and 2 are depicted.

FIG. 17A-17D shows the MutComputeX pipeline. FIG. 17A shows the A53 microenvironment inputs for MutCompute (top), which does not include non-protein atoms, and for MutComputeX (bottom), which includes both ligand and cofactor atoms (PDB: 8UKE). FIG. 17B The microenvironment is voxelized into seven elemental and two physical channels. All halogen atoms are combined into a single channel. FIG. 17C An overview of MutComputeX residual neural network architectures. FIG. 17D Workflow using MutComputeX for enzyme engineering. In the A53 masked microenvironment that is shown, the light blue spheres represents the masked alanine, the norbelladine ligand is shown in aqua, protein residues are shown in gray, and S-adenosyl-homocysteine (SAH) is shown in pink.

FIG. 18A-18E shows a crystal structure of an example engineered norbelladine 4′-O-methyltransferase. FIG. 18A shows the global structure of Nb4OMT^{E36P/G40E/A53M} solved in 2.4A resolution. One dimer is colored blue while the other dimer is transparent. FIG. 18B shows a comparison of N-termini of Nb4OMT crystal structure (orange) and wild-type Nb4OMT AlphaFold2 structure (blue). FIG. 18C shows local context of the A53M mutant residue. FIG. 18D shows a local context of the E36P and G40E mutant residues. Black arrow indicates the shift of glutamate from position 36 to position 40. FIG. 28E shows an active site context of Nb4OMT^{E36P/G40E/A53M} in complex with SAH and docked with norbelladine. For FIGS. 17A, 17C, 17D, and 17E, the color coding is as follows-calcium ions: green, s-adenosylhomocysteine: purple, mutant residues: orange, non-mutant residues: gray, docked norbelladine: seafoam green, interactions: yellow dashed lines.

FIG. 19 shows MutComputeX predictions for the 53 position with and without ligand docking. AF and Exp indicate that either the AlphaFold2 structure model or the experimentally-determined crystal structure were passed as inputs to MutComputeX, respectively. “Apo” indicates that the structure was not docked with any norbelladine using GNINA1.0, where “Holo” indicates that the structure was docked.

FIG. 20A-20C shows steady state kinetics of Nb4OMT variants. Steady state experiments were set up as described in the methods section. Plots show the best fits by simulation in KinTek Explorer for the concentration of product at each substrate concentration as a function of time. Red, green, blue, yellow, cyan, and purple lines show data at 15.625, 31.25, 62.5, 125, 250, and 500 μM of substrate, respectively. FIG. 20A is a plot of WT, FIG. 20B is a plot of A53M, and FIG. 20C is a plot of Triple.

FIG. 21 shows size exclusion chromatography profiles of purified Nb4OMT variants.

FIG. 22 shows an omit Fo-Fc map (contoured at 3.00) shown as green meshes superimposed on the stick and sphere model of S-adenosyl-L-homocysteine (purple) and Ca²⁺ ion.

FIG. 23 shows alignment of the crystal structure and AlphaFill model of Nb4OMT Color coding is as follows: AlphaFill model: purple; Crystal structure: blue; Ca²⁺ ions: green; SAH cofactor in the AlphaFill model: red; SAH cofactor in the Crystal structure: yellow. The norbelladine substrate was not included in the AlphaFill model.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Reference also is made herein to peptides, polypeptides, proteins and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

As disclosed herein, exemplary peptides, polypeptides, proteins may comprise, consist essentially of, or consist of any reference amino acid sequence disclosed herein, or variants of the peptides, polypeptides, and proteins may comprise, consist essentially of, or consist of an amino acid sequence having at least about 80%, 90%, 95%, 96%, 97%, 98%, or 99% sequence identity to any amino acid sequence disclosed herein. Variant peptides, polypeptides, and proteins may include peptides, polypeptides, and proteins having one or more amino acid substitutions, deletions, additions and/or amino acid insertions relative to a reference peptide, polypeptide, or protein. Also disclosed are nucleic acid molecules that encode the disclosed peptides, polypeptides, and proteins (e.g., polynucleotides that encode any of the peptides, polypeptides, and proteins disclosed herein and variants thereof).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full-length polynucleotide or full-length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

Fusion proteins and fusion polynucleotides are also contemplated herein. A “fusion protein” refers to a protein formed by the fusion of at least one peptide, polypeptide, protein or variant thereof as disclosed herein to at least one molecule of a heterologous peptide, polypeptide, protein or variant thereof. The heterologous protein(s) may be fused at the N-terminus, the C-terminus, or both termini. A fusion protein comprises at least a fragment or variant of the heterologous protein(s) that are fused with one another, preferably by genetic fusion (i.e., the fusion protein is generated by translation of a nucleic acid in which a polynucleotide encoding all or a portion of a first heterologous protein is joined in-frame with a polynucleotide encoding all or a portion of a second heterologous protein). The heterologous protein(s), once part of the fusion protein, may each be referred to herein as a “portion”, “region” or “moiety” of the fusion protein.

A fusion polynucleotide refers to the fusion of the nucleotide sequence of a first polynucleotide to the nucleotide sequence of a second heterologous polynucleotide (e.g., the 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

“Homology” refers to sequence similarity or, interchangeably, sequence identity, between two or more polypeptide sequences or polynucleotide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (Sec, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit one or more biological activities associated with binding a ligand and/or binding DNA at a specific binding site.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (Sec, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 1 3, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The term “cDNA” as used herein refers to all polynucleotides that share the arrangement of sequence elements found in native mature mRNA species, where sequence elements are exons and 5′ and 3′ non-coding regions. Normally mRNA species have contiguous exons, with the intervening introns, when present, being removed by nuclear RNA splicing, to create a continuous open reading frame encoding the protein.

The term “homologous” as used herein in reference to polynucleotides and polynucleotide sequences is intended to mean obtainable from the same biological species, i.e. a first and second polynucleotide sequence are homologous when they are obtainable from the same biological species, and conversely, a first and second polynucleotide sequence are non-homologous when they are obtainable or obtained from two different biological species.

The term “in vitro” as used herein refers to the performance of a biochemical reaction outside a living cell, including, for example, in a microwell plate, a tube, a flask, a tank, a reactor and the like, for example a reaction to form an alkaloid compound.

The term “in vivo” as used herein refers to the performance of a biochemical reaction within a living cell, including, for example, a microbial cell, or a plant cell, for example to form an alkaloid compound.

The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity, however in each case less than 100%, compared to a reference polynucleotide sequence using the programs.

Norbelladine 4′-O-Methyltransferase (EC 2.1.1.336) is an enzyme involved in Amaryllidaceae alkaloid biosynthesis that utilizes the co-substrate S-adenosyl methionine to methylate norbelladine to form 4′-O-methylnorbelladine. The terms “Norbelladine 4′-O-Methyltransferase”, also referred to herein as “Nb4OMT”, which may be used interchangeably herein, refer to any and all enzymes comprising a sequence of amino acid residues which is (i) substantially identical to the amino acid sequences constituting any Nb4OMT polypeptide set forth herein, including, for example, SEQ. ID NO: 3, or variants thereof, such as SEQ ID NOS: 4-8, 17, or 18, or (ii) encoded by a nucleic acid sequence capable of hybridizing under at least moderately stringent conditions to any nucleic acid sequence encoding any Nb4OMT polypeptide set forth herein, but for the use of synonymous codons.

“Transformation” describes a process by which exogenous DNA is introduced into a recipient cell. Transformation may occur under natural or artificial conditions according to various methods well known in the art and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

The disclosed technology relates to “biosensors.” As disclosed herein, a “biosensor” is a molecule or a system of molecules that can be used to bind to a ligand (or target molecule) and provide a detectable response based on binding the ligand. In some cases, “biosensors” may be referred to as “molecular switches.” Biosensors and molecular switches are disclosed in the art. (Scc, e.g., Ostermeier, Protein Eng. Des. Sel. 2005 August; 18 (8): 359-64; Wright et al., Curr. Opin. Chem. Biol. 2007 June; 11 (3): 342-6; Roberts, Chem. Biol. 2004 Nov.; 11 (11): 1475-6; and U.S. Pat. Nos. 8,771,679; 8,679,753; and 8,338,138; the contents of which are incorporated herein by reference in their entireties). Biosensors and molecular switches have been utilized in recombinant microorganisms. (See, e.g., Rogers et al., Curr. Opin. Biotechnol. 2016 Mar. 18; 42:84-91; and U.S. Published Application Nos. 2010/0242345 and 2013/0059295; the contents of which are incorporated herein by reference in their entireties).

A “substrate-promiscuous regulator” refers to any protein with the ability to bind to and report on the concentration of more than one chemical. For instance, the naturally occurring promiscuous regulators from which the biosensors disclosed herein are derived has been reported to bind to several different unrelated chemicals (Yamasaki, S., Nikaido, E., Nakashima, R. et al. Nat Commun 2013) Another common feature of substrate-promiscuous regulators is that the chemicals they bind are often structurally unrelated, but share some common general feature, such as being hydrophobic.

The systems, components, and methods disclosed herein may be utilized for sensing a ligand or a substrate or a metabolite in a cell or a reaction mixture. The disclosed systems, components, and methods typically include and/or utilize an engineered (non-naturally occurring) biosensor. The biosensors disclosed herein bind the ligand and modulate expression of an output signal, such as a reporter gene, which can be operably linked to a promoter that is engineered to include specific binding sites for the input signal. The difference in expression of the output signal in the presence of the ligand versus expression of the output signal in the absence of the ligand can be correlated to the concentration of the ligand in a reaction mixture.

As used herein, “modulating expression” may include “repressing expression” and/or “inhibiting expression,” and “modulating expression may include “de-repressing expression” and/or “activating expression.” As such, in some embodiments, when the biosensor is not bound to a ligand, the biosensor may repress expression and/or inhibit expression from a promoter that is engineered to include specific binding sites for the DNA-binding protein, and when the biosensor is bound to the ligand the biosensor may de-repress and/or activate expression from the promoter. De-repression and/or activation of the expression of the reporter gene then can be correlated with the presence of the ligand. In other embodiments, when the biosensor is bound to a ligand, the biosensor may repress expression and/or inhibit expression, and when the biosensor is not bound to the ligand the biosensor may de-repress expression and/or activate expression. A decrease in expression of the reporter gene then can be correlated with the presence of the ligand.

The disclosed biosensors, systems, and methods may be utilized and/or performed using any suitable cell. Suitable cells may include prokaryotic cells and eukaryotic cells. It can also be carried out in a cell-free environment.

### General Description of Invention

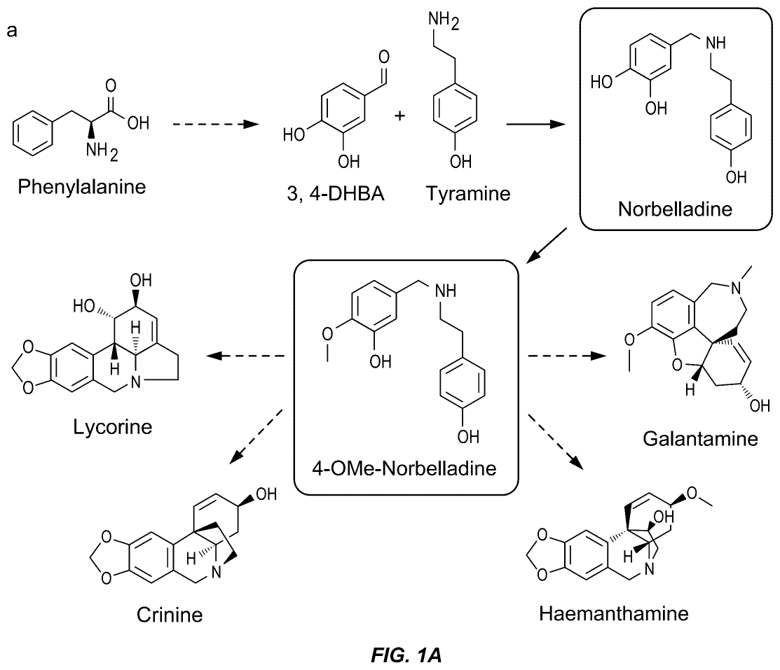

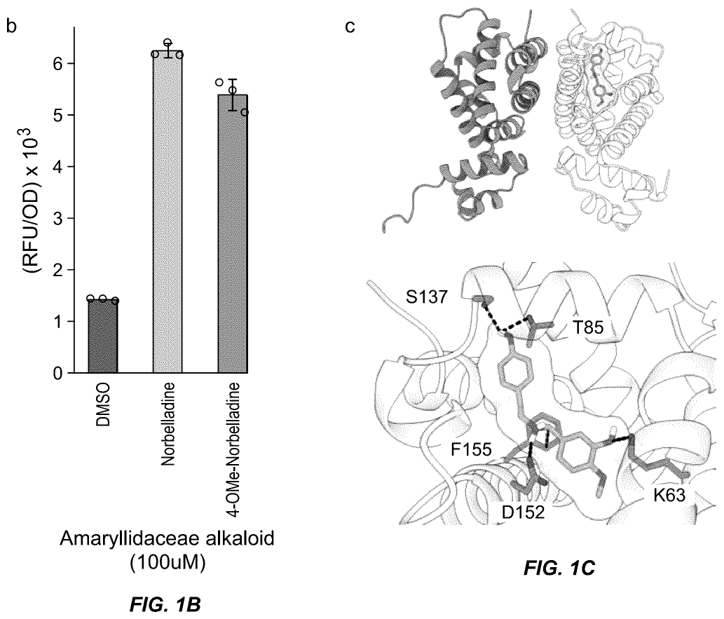

A major challenge to achieving industry-scale biomanufacturing of therapeutic alkaloids is the slow process of biocatalyst engineering. Amaryllidaceae alkaloids, such as the Alzheimer's medication galantamine, are complex plant secondary metabolites with recognized therapeutic value. Due to their difficult synthesis they are regularly sourced by extraction and purification from low-yielding plants, including the wild daffodil Narcissus pseudonarcissus. Engineered biocatalytic methods have the potential to stabilize the supply chain of amaryllidaceae alkaloids. Disclosed herein is an engineered methyltransferase, wherein said methyltransferase can methylate norbelladine to form 4-O'Methylnorbelladine. As can be seen in FIG. 1, this is an important step in the formation of several amaryllidaceae alkaloids, including galantamine, haemanthamine, crinine, and lycorine.

Also disclosed is a highly efficient biosensor for biocatalyst development, which has been applied to engineer amaryllidaceae alkaloid production in Escherichia coli (Example 1). Directed evolution was used to develop a highly sensitive (EC₅₀=20 uM) and specific biosensor for the key amaryllidaceae alkaloid branchpoint 4-O'Methylnorbelladine. A machine learning model (MutComputeX) was subsequently developed and used to generate activity-enriched variants of a plant methyltransferase, which were rapidly screened with the biosensor. Functional enzyme variants were identified that yielded a 60% improvement in product titer, 17-fold reduced remnant substrate, and 3-fold lower off-product regioisomer formation (Example 1).

### Engineered Methyltransferases

Disclosed herein are non-naturally occurring methyltransferases, wherein said methyltransferases can methylate norbelladine to form 4-O'Methylnorbelladine. These methyltransferases can be, for example, Norbelladine 4′-O-Methyltransferases (Nb4OMT).

These engineered methyltransferases have advantages over native norbelladine methyltransferases. (It is noted that an example of native norbelladine methyltransferase is represented by SEQ ID NO: 3.) For example, the engineered methyltransferases of the invention can form less 3-O'Methylnorbelladine (an undesirable byproduct of amaryllidaceae alkaloid synthesis) compared to a native norbelladine methyltransferase. By “less” is meant 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% less 3-O'Methylnorbelladine is produced.

In another example of an advantage over native norbelladine methyltransferase, the engineered methyltransferases of the present invention can be more active than the native norbelladine methyltransferase. By “more active” is meant that a higher percentage of conversion from norbelladine to 4-O'Methylnorbelladine takes place. The engineered methyltransferases can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% more active compared to native, or non-engineered norbelladine methyltransferase.

The engineered methyltransferase disclosed herein can be about 80, 81, 82, 83, 84, 85, 86, 87, 88, 89, 90, 91, 92, 93, 94, 95, 96, 97, 98, or 99% identical to SEQ ID NO: 3. Viewed another way, the engineered methyltransferase can have 1, 2, 3, 4, 5, 6, 7, 8, 9, 10, 11, 12, 13, 14, 15, 16, 17, 18, 19, or 20 or more amino acid variations when compared to SEQ ID NO 3. Such variations can be substitutions, deletions, or insertions. For example, disclosed herein is an engineered methyltransferase comprising any of SEQ ID NOS: 4-8 or 17 or 18. SEQ ID NOS: 4-8 and 17 and 18 vary from SEQ ID NO: 3 in that SEQ ID NO: 4 comprises a mutation of A53M; SEQ ID NO: 5 comprises a mutation of S159E; SEQ ID NO: 6 comprises a mutation of V203E; SEQ ID NO: 7 comprises a mutation of H17K; SEQ ID NO: 8 comprises mutations of E36P and G40E, SEQ ID NO: 17 comprises a mutation of H17R, and SEQ ID NO: 18 comprises a mutation of E36P, G40E, and A53M. It is noted that any of SEQ ID NOS: 4-8 and 17-18 can vary by 90%, 91%, 92%; 93%, 94%, 95%, 96%, 97%, 98%, 99%, or any amount above, below or in between these amounts. In a specific example, although other amino acid sequences can vary, with respect to SEQ ID NO: 4, position 53A does not vary; for SEQ ID NO: 5, position 159E does not vary; for SEQ ID NO: 6, position 203E does not vary; for SEQ ID NO: 7, position 17K does not vary; for SEQ ID NO: 8, neither position 36P nor position 40E vary, for SEQ ID NO: 17, 17R does not vary, and SEQ ID NO: 18, none of E36P, G40E, and A53M. vary.

Also disclosed herein is a nucleic acid encoding the methyltransferases disclosed herein, as well as host cells. The host cells may also be modified to possess one or more genetic alterations (nucleic acids) to accommodate the heterologous coding sequences. Alterations of the native host genome include, but are not limited to, modifying the genome to reduce or ablate expression of a specific enzyme that may interfere with the desired pathway. The presence of such native enzymes may rapidly convert one of the intermediates or final products of the pathway into a metabolite or other compound that is not usable in the desired pathway. Thus, if the activity of the native enzyme were reduced or altogether absent, the produced intermediates would be more readily available for incorporation into the desired product. Genetic alterations may also include modifying the promoters of endogenous genes to increase expression and/or introducing additional copies of endogenous genes. Examples of this include the construction/use of strains which overexpress the endogenous yeast NADPH-P450 reductase CPR1 to increase activity of heterologous P450 enzymes, or the overexpression of the endogenous S-adenosylmethionine synthetase for higher S-adenosylmethionine cofactor generation. In addition, endogenous enzymes such as ARO8, 9, and 10, which are directly involved in the synthesis of intermediate metabolites, may also be overexpressed.

Alternatively, the methyltransferase, methods of using the methyltransferase, and systems and kits which make use of the methyltransferase can be done in a cell-free (in vitro) environment. One of skill in the art will readily appreciate how this can be done.

The heterologous coding sequences of the present invention are sequences that encode enzymes, either wild-type or equivalent sequences, which are normally responsible for the production of amaryllidaceae alkaloids (also referred to herein as AA) in plants. The enzymes for which the heterologous sequences code can be any of the enzymes in the AA pathway and can be from any known source. The choice and number of enzymes encoded by the heterologous coding sequences for the particular synthetic pathway should be chosen based upon the desired product. For example, the host cells of the present invention may comprise at least 1, 2, 3, 4, 5, 6, 7, 8, 9, 10, 11, 12, 13, 14, 15 or more heterologous coding sequences (nucleic acids). Methods of preparing AAs using these modified cells are discussed in more detail below.

The amaryllidaceae alkaloids represent a large and still expanding group of isoquinoline alkaloids, usually classified into nine skeleton types whose representative compounds are: norbelladine, lycorine, homolycorine, crinine, hacmanthamine, narciclasine, tazettine, montanine and galanthamine (Guo et al., Natural Product Communications; 2014 Vol. 9, No. 8, pages 1081-1086). These AAs are examples of those which can be synthesized using the norbelladine methyltransferase described herein include.

### Methods of Preparing Amaryllidaceae Alkaloids

Disclosed herein is a method of preparing amaryllidaceae alkaloid (AA) compositions wherein the AA composition requires methylation of norbelladine to form 4-O'Methylnorbelladine. This method can comprise the following steps: culturing a host cell under suitable conditions, wherein the host cell comprises nucleic acid encoding a non-naturally occurring methyltransferase; exposing the methyltransferase to norbelladine; and allowing the methyltransferase to methylate norbelladine, thereby producing a methylated composition of interest.

As mentioned above, disclosed herein is a host cell that produces one or more AAs of interest. Any convenient cells may be utilized in the subject host cells and methods. In some cases, the host cells are non-plant cells. In some instances, the host cells may be characterized as microbial cells. In certain cases, the host cells are mammalian cells, bacterial cells, or yeast cells.

Host cells of interest include, but are not limited to, bacterial cells, such as Bacillus subtilis, Escherichia coli, Streptomyces and Salmonella typhimuium cells, and yeast cells such as Saccharomyces cerevisiae, Schizosaccharomyces pombe, and Pichia pastoris cells. In some embodiments, the host cells are yeast cells or E. coli cells. In some cases, the host cell is a yeast cell. In some instances, the host cell is from a strain of yeast engineered to produce a AA of interest. In certain embodiments, the yeast cells may be of the species Saccharomyces cerevisiae (S. cerevisiae). In certain embodiments, the yeast cells may be of the species Schizosaccharomyces pombe. In certain embodiments, the yeast cells may be of the species Pichia pastoris. Yeast is of interest as a host cell because cytochrome P450 proteins, which are involved in some biosynthetic pathways of interest, are able to fold properly into the endoplasmic reticulum membrane so that their activity is maintained.

Yeast strains of interest that find use in the invention include, but are not limited to, CEN.PK (Genotype: MATa/α ura3-52/ura3-52 trp1-289/trp1-289 leu2-3_112/leu2-3_112 his3 Δ1/his3 Δ1 MAL2-8C/MAL2-8C SUC2/SUC2), S288C, W303, D273-10B, X2180, A364A, Σ1278B, AB972, SKI, and FL100. In certain cases, the yeast strain is any of S288C (MATα; SUC2 mal mel gal2 CUP1 flo1 flo8-1 hap1), BY4741 (MATα; his3Δ1; leu2Δ0; met15Δ0; ura3Δ0), BY4742 (MATα; his3Δ1; leu2Δ0; lys2Δ0; ura3Δ0), BY4743 (MATa/MATα; his3Δ1/his3Δ1; leu2Δ0/leu2Δ0; met15Δ0/MET15; LYS2/lys2Δ0; ura3Δ0/ura3Δ0), and WAT11 or W(R), derivatives of the W303-B strain (MATa; ade2-1; his3-11,-15; leu2-3,-112; ura3-1; canR; cyr+) which express the Arabidopsis thaliana NADPH-P450 reductase ATR1 and the yeast NADPH-P450 reductase CPR1, respectively. In another embodiment, the yeast cell is W303alpha (MATα; his3-11, 15 trp1-1 leu2-3 ura3-1 ade2-1). The identity and genotype of additional yeast strains of interest may be found at EUROSCARF (web.uni-frankfurt.de/fb15/mikro/euroscarf/col_index.html).

The host cells may be engineered to include one or more modifications (such as two or more, three or more, four or more, five or more, or even more modifications) that provide for the production of AAs of interest. In some cases, by modification is meant a genetic modification, such as a mutation, addition, or deletion of a gene or fragment thereof, or transcription regulation of a gene or fragment thereof. In some cases, the one or more (such as two or more, three or more, or four or more) modifications is selected from: a feedback inhibition alleviating mutation in a biosynthetic enzyme gene native to the cell; a transcriptional modulation modification of a biosynthetic enzyme gene native to the cell; an inactivating mutation in an enzyme native to the cell; and a heterologous coding sequence that encodes an enzyme. A cell that includes one or more modifications may be referred to as a modified cell.

A modified cell may overproduce one or more precursor AA, AA, or modified AA molecules. By overproduce is meant that the cell has an improved or increased production of a AA molecule of interest relative to a control cell (e.g., an unmodified cell). By improved or increased production is meant both the production of some amount of the AA of interest where the control has no AA precursor production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some AA of interest production.

In some cases, the host cell is capable of producing an increased amount of tetrahydropapaverine relative to a control host cell that lacks the modified methyltransferase described herein In certain instances, the increased amount of tetrahydropapaverine is about 10% or more relative to the control host cell, such as about 20% or more, about 30% or more, about 40% or more, about 50% or more, about 60% or more, about 80% or more, about 100% or more, 2-fold or more, 5-fold or more, or even 10-fold or more relative to the control host cell.

In some embodiments of the host cell, when the cell includes one or more heterologous coding sequences that encode one or more enzymes, it includes at least one additional modification selected from the group consisting of: a feedback inhibition alleviating mutations in a biosynthetic enzyme gene native to the cell; a transcriptional modulation modification of a biosynthetic enzyme gene native to the cell; and an inactivating mutation in an enzyme native to the cell. In certain embodiments of the host cell, when the cell includes one or more feedback inhibition alleviating mutations in one or more biosynthetic enzyme genes native to the cell, it includes a least one additional modification selected from the group consisting of: a transcriptional modulation modification of a biosynthetic enzyme gene native to the cell; an inactivating mutation in an enzyme native to the cell; and a heterologous coding sequence that encode an enzyme. In some embodiments of the host cell, when the cell includes one or more transcriptional modulation modifications of one or more biosynthetic enzyme genes native to the cell, it includes at least one additional modification selected from the group consisting of: a feedback inhibition alleviating mutation in a biosynthetic enzyme gene native to the cell; an inactivating mutation in an enzyme native to the cell; and a heterologous coding sequence that encodes an enzyme. In certain instances of the host cell, when the cell includes one or more inactivating mutations in one or more enzymes native to the cell, it includes at least one additional modification selected from the group consisting of: a feedback inhibition alleviating mutation in a biosynthetic enzyme gene native to the cell; a transcriptional modulation modification of a biosynthetic enzyme gene native to the cell; and a heterologous coding sequence that encodes an enzyme.

Also disclosed herein is a kit comprising: a non-naturally occurring methyltransferase, wherein said methyltransferase can methylate norbelladine. The kit can include one or more additional components as outlined above.

### Engineered Biosensors

Disclosed herein is a biosensor for detecting 4-O'Methylnorbelladine, wherein the biosensor comprises an engineered substrate-promiscuous regulator, wherein said substrate-promiscuous regulator has been engineered to interact more efficiently with 4-O'Methylnorbelladine than does a naturally occurring substrate promiscuous regulator; and further wherein the biosensor is engineered to provide an output signal, wherein said output signal is generated when the biosensor interacts with 4-O'Methylnorbelladine.

Designing genetic biosensors is known in the art (Hossain et al., “Genetic Biosensor Design for Natural Product Biosynthesis in Microorganisms, Trends in Biotechnology 38 (7), p797-810, April 2020, herein incorporated by reference in its entirety for its teaching concerning biosensors). A genetic biosensor is made up of a sensing device and a transduction device, which can be formed by genetic parts. The sensing device serves to detect the existence of an input signal such as a ligand. It contains a TF (transcriptional activator, transcriptional repressor) consisting of a DNA-binding domain (DBD) and a ligand-binding domain (LBD), or an element such as a riboswitch comprising an RNA aptamer. The transduction device translates the input signal into an output signal (e.g., fluorescence, colorimetry, or a genetic trait, such as antibiotic resistance, for example). It contains a reporter gene or pathway genes. The sensing device can be functionally linked to the transduction device through the binding of the input signal to a TF or a riboswitch, for example, activating or repressing transcription or translation of genes of interest. In TF-based biosensors, mediated by DBD and/or LBD, transcriptional activators activate transcription of reporter genes by binding to promoters, and transcriptional repressors repress transcription of actuator genes by dissociating from promoters or binding to a co-repressing ligand in an allosteric manner.

Substrate-promiscuous regulators can be used as a starting platform to engineer biosensors that are specific for a certain ligand (referred to alternatively herein as a target). Because these promiscuous regulators can have a high degree of evolvability, they can be engineered with relative case to be specific for a ligand. In one example, a person of skill in the art can identify a potential substrate-promiscuous regulator that can be engineered for a specific ligand by identifying a substrate promiscuous regulator that shows some degree of affinity for the ligand, then evolving the substrate-promiscuous regulator through mutation to create a biosensor with a much higher degree of specificity for the ligand than the naturally occurring regulator. For example, the engineered substrate-promiscuous regulator can be 2, 3, 4, 5, 6, 7, 8, 9, 10, 20, 30, 40, 50, 60, 70, 80, 90, or 100 times (or more) more efficient at interacting with the ligand than the naturally occurring regulator.

In one example, the substrate-promiscuous regulator disclosed herein can be a genetically engineered regulator, such as a multidrug resistance regulator. Regulators in this family contain a poly-specific substrate binding pocket that enables them to bind and extrude a diverse array of compounds from the periplasm to the exterior of the cell, including the majority of clinically used antibiotics (Aron et al., Res Microbiol. 2018 Sep.-Oct.; 169 (7-8): 393-400). In order to have utility in microbial engineering for plant metabolites, sensors must be highly specific and sensitive to their target molecule to avoid false positives and report on low-activity pathways, respectively, making multidrug resistance regulators an ideal candidate for engineered biosensors. In a specific example, the substrate-promiscuous regulator can comprise a large hydrophobic binding pocket that contains numerous aromatic residues, such as phenylalanine, tyrosine, and/or tryptophan.

An example of naturally occurring multidrug resistance regulator that can be used as a platform from which to engineer the biosensors of the present invention includes, but is not limited to, RamR (WP_000113609.1, represented by SEQ ID NO: 9).

The engineered biosensor can have 90, 91, 92, 93, 94, 95, 96, 97, 98, or 99% identity with a naturally occurring substrate-promiscuous regulator. Viewed another way, the engineered biosensor can vary from a naturally occurring substrate-promiscuous regulator by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. This variation can be in the form of an insertion, deletion, or substitution, or a combination of two or more of these. Given the teachings disclosed herein, one of skill in the art can readily engineer a naturally occurring substrate promiscuous regulator to be highly specific for a desired target molecule (ligand). Specifically, the engineered biosensor of the present invention can vary with regard to SEQ ID NO: 9.

For example, disclosed herein is an engineered methyltransferase comprising any of SEQ ID NOS: 10-16. SEQ ID NOS: 10-16 vary from SEQ ID NO: 9 in that SEQ ID NO: 10 comprises a mutation of L133T, C134E, and S127T; SEQ ID NO: 11 comprises a mutation of K63T and M70T; SEQ ID NO: 12 comprises a mutation of K63R and M70T; SEQ ID NO: 13 comprises a mutation of K63T, L66M, C134D, and S137G; and SEQ ID NO: 14 comprises mutations of K63T, L66M, C134D, and S137N; SEQ ID NO: 15 comprises mutations of K63T, L66M, C134E, and S137D; SEQ ID NO: 16 comprises mutations of K63T, L66M, C134N, and S137G.

By way of further specific example, the biosensor of the present invention can comprise at least one substitution of K63T and/or L66M compared to native RamR (as represented by SEQ ID NO: 9). In another embodiment, the biosensor can comprise a substitution at C134D compared to native RamR (as represented by SEQ ID NO: 9). It is noted that any of SEQ ID NOS: 10-16 can vary by 90%, 91%, 92%; 93%, 94%, 95%, 96%, 97%, 98%, 99%, or any amount above, below or in between these amounts. In a specific example, although other amino acid sequences can vary, with respect to SEQ ID NO: 10, the sequence does not vary at positions 133T, 134E, or 127T. With respect to SEQ ID NO: 11, the sequence does not vary at positions 63T or 66M. With respect to SEQ ID NO: 12 the sequence does not vary at positions 63R or 70T. With respect to SEQ ID NO: 13, the sequence does not vary at positions 63T, 66M, 134D, or 137G. With respect to SEQ ID NO: 14, the sequence does not vary at positions 63T, 66M, 134D, or 137N. With respect to SEQ ID NO: 15, the sequence does not vary at positions 63T, 66M, 134E, or 137D. With respect to SEQ ID NO: 16, the sequence does not vary at positions 63T, 66M, 134N, or 137G.

With respect to the biosensor, the “input signal” can be 4-O'Methylnorbelladine. The “output signal” refers to any detectable signal that indicates the presence of the input signal. For example, the output signal can be the expression, or repression of expression, of a gene. The output signal can be fluorescence, luminescence, or a colorimetric signal. Examples include, but are not limited to, bioluminescent proteins such as a luciferase, a ß-galactosidase, a lactamase, a horseradish peroxidase, an alkaline phosphatase, a β-glucuronidase or a β-glucosidase. Examples of luciferases include, but are not necessarily limited to, a Renilla luciferase, a Firefly luciferase, a Coelenterate luciferase, a North American glow worm luciferase, a click beetle luciferase, a railroad worm luciferase, a bacterial luciferase, a Gaussia luciferase, Acquorin, an Arachnocampa luciferase, or a biologically active variant or fragment of any one, or chimera of two or more, thereof. The output signal can be fluorescent. Examples include, but are not limited to, green fluorescent protein (GFP), blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Venus, mOrange, Topaz, GFPuv, destabilized EGFP (dEGFP), destabilized ECFP (dECFP), destabilised EYFP (dEYFP), HcRed, t-HcRed, DsRed, DsRed2, t-dimer2, t-dimer2 (12), mRFP1, pocilloporin, Renilla GFP, Monster GFP, paGFP, Kaede protein or a Phycobiliprotein, or a biologically active variant or fragment of any one thereof. The fluorescent molecule can also be a non-protein. Examples include, but are not necessarily limited to, an Alexa Fluor dye, Bodipy dye, Cy dye, fluorescein, dansyl, umbelliferone, fluorescent microsphere, luminescent microsphere, fluorescent nanocrystal, Marina Blue, Cascade Blue, Cascade Yellow, Pacific Blue, Oregon Green, Tetramethylrhodamine, Rhodamine, Texas Red, rare earth element chelates, or any combination or derivatives thereof.

The input signal (such as 4-O'Methylnorbelladine) can be converted to the output signal by a transduction system. The transduction system can comprise a transcriptional activator or transcriptional repressor of the output signal. For example, the transcriptional activator or transcriptional repressor is encoded with the engineered substrate promiscuous regulator. The transduction system can further comprise a promoter or operator and a regulator. Methods of using transduction systems in a biosensor are known to those of skill in the art and can be deployed with the method disclosed herein. Interaction between the input signal and the transduction system can be covalent or non-covalent.

### Cells and Plasmids Comprising Engineered Biosensors

The disclosed biosensors, systems, and methods may be utilized and/or performed in vitro. In other words, the biosensors, systems, and methods disclosed herein can take place in a cell-free environment. One of ordinary skill in the art will understand how this can be done. Alternatively, the biosensors, systems, and methods disclosed herein can be carried out using any suitable cell. For example, the biosensors disclosed herein can be integrated into a host genome, or can be in a plasmid. Disclosed herein is a host cell that produces one or more ligands, such as an AA. Any convenient type of host cell may be utilized in producing the ligand, see, e.g., US2008/0176754, the disclosure of which is incorporated by reference in its entirety.

Any convenient cells may be utilized in the subject host cells and methods. In some cases, the host cells are non-plant cells. In certain cases, the host cells are mammalian cells, bacterial cells or yeast cells. Host cells of interest include, but are not limited to, bacterial cells, such as Bacillus subtilis, Escherichia coli, Streptomyces and Salmonella typhimuium cells. In some embodiments, the host cells are yeast cells or E. coli cells. In certain embodiments, the yeast cells can be of the species Saccharomyces cerevisiae (S. cerevisiae).

The term “host cells,” as used herein, are cells that harbor one or more heterologous coding sequences which encode activity (ies) that enable the host cells to produce desired ligands e.g., as described herein. The heterologous coding sequences could be integrated stably into the genome of the host cells, or the heterologous coding sequences can be transiently inserted into the host cell. As used herein, the term “heterologous coding sequence” is used to indicate any polynucleotide that codes for, or ultimately codes for, a peptide or protein or its equivalent amino acid sequence, e.g., an enzyme, that is not normally present in the host organism and can be expressed in the host cell under proper conditions. As such, “heterologous coding sequences” includes multiple copies of coding sequences that are normally present in the host cell, such that the cell is expressing additional copies of a coding sequence that are not normally present in the cells. The heterologous coding sequences can be RNA or any type thereof, e.g., mRNA, DNA or any type thereof, e.g., cDNA, or a hybrid of RNA/DNA. Examples of coding sequences include, but are not limited to, full-length transcription units that comprise such features as the coding sequence, introns, promoter regions, 3′-UTRs and enhancer regions.

As used herein, the term “heterologous coding sequences” also includes the coding portion of the peptide or enzyme, i.e., the cDNA or mRNA sequence, of the peptide or enzyme, as well as the coding portion of the full-length transcriptional unit, i.e., the gene comprising introns and exons, as well as “codon optimized” sequences, truncated sequences or other forms of altered sequences that code for the enzyme or code for its equivalent amino acid sequence, provided that the equivalent amino acid sequence produces a functional protein. Such equivalent amino acid sequences can have a deletion of one or more amino acids, with the deletion being N-terminal, C-terminal or internal. Truncated forms are envisioned as long as they have the catalytic capability indicated herein. Fusions of two or more enzymes are also envisioned to facilitate the transfer of metabolites in the pathway, provided that catalytic activities are maintained.

Operable fragments, mutants or truncated forms may be identified by modeling and/or screening. This is made possible by deletion of, for example, N-terminal, C-terminal or internal regions of the protein in a step-wise fashion, followed by analysis of the resulting derivative with regard to its activity for the desired reaction compared to the original sequence. If the derivative in question operates in this capacity, it is considered to constitute an equivalent derivative of the enzyme proper.

The host cells may also be modified to possess one or more genetic alterations to accommodate the heterologous coding sequences. Alterations of the native host genome include, but are not limited to, modifying the genome to reduce or ablate expression of a specific protein that may interfere with the desired pathway. The presence of such native proteins may rapidly convert one of the intermediates or final products of the pathway into a metabolite or other compound that is not usable in the desired pathway. Thus, if the activity of the native enzyme were reduced or altogether absent, the produced intermediates would be more readily available for incorporation into the desired product.

Such gene deletions may lead to improved ligand production. The expression of cytochrome P450s may induce the unfolded protein response and may cause the ER to proliferate. Deletion of genes associated with these stress responses may control or reduce overall burden on the host cell and improve pathway performance. Genetic alterations may also include modifying the promoters of endogenous genes to increase expression and/or introducing additional copies of endogenous genes. Examples of this include the construction/use of strains which overexpress the endogenous yeast NADPH-P450 reductase CPR1 to increase activity of heterologous P450 enzymes. In addition, endogenous enzymes such as ARO8, 9, and 10, which are directly involved in the synthesis of intermediate metabolites, may also be overexpressed.

In some instances, the expression of each type of ligand is increased through additional gene copies (i.e., multiple copies), which increases intermediate accumulation and ultimately ligand production. Embodiments of the present invention include increased ligand production in a host cell through simultaneous expression of multiple species variants of a single or multiple enzymes. In some cases, additional gene copies of a single or multiple enzymes are included in the host cell. Any convenient methods may be utilized in including multiple copies of a heterologous coding sequence for an enzyme in the host cell.

In some embodiments, the host cell includes multiple copies of a heterologous coding sequence for an enzyme, such as 2 or more, 3 or more, 4 or more, 5 or more, or even 10 or more copies. In certain embodiments, the host cell include multiple copies of heterologous coding sequences for one or more enzymes, such as multiple copies of two or more, three or more, four or more, etc. In some cases, the multiple copies of the heterologous coding sequence for an enzyme are derived from two or more different source organisms as compared to the host cell. For example, the host cell may include multiple copies of one heterologous coding sequence, where each of the copies is derived from a different source organism. As such, each copy may include some variations in explicit sequences based on inter-species differences of the enzyme of interest that is encoded by the heterologous coding sequence.

### Kits and Proteins/Nucleic Acids

Also disclosed herein is a kit, wherein the kit comprises a 4-O'Methylnorbelladine biosensor comprising an engineered substrate-promiscuous regulator, wherein said substrate-promiscuous regulator has been engineered to interact more efficiently with 4-O'Methylnorbelladine than does the naturally occurring substrate promiscuous regulator, such as SEQ ID NO: 9. Such biosensors are described in detail above. The kit disclosed herein can be customized to be specific for a given ligand, for example, or for a series of different ligands.

The kit can comprise a plasmid encoding the engineered biosensor, or a cell with these elements integrated within its genome. The cell can have the biosensor and corresponding elements needed for expression engineered into the cell, or, alternatively, the cell can be transformed with a plasmid. The kit can further comprise components needed for detection of expression of a target molecule, such as the individual biosensor proteins themselves. The protein sensors may be purified individually and used outside a cellular context. One of skill in the art will understand what components can be included in such a kit.

### EXAMPLES

### Example 1: Synthetic Microbial Sensing and Biosynthesis of Amaryllidaceae Alkaloids

Disclosed herein is the development of custom biosensors with machine learning-guided protein design as a paradigm for rapidly prototyping and improving new pathways. In particular, in order to improve microbial fermentation of the branchpoint AA 4-O'Methylnorbelladine (4NB) a generalist transcription factor, RamR, was evolved into a highly sensitive biosensor for 4NB that precisely discriminates against the non-methylated precursor norbelladine, and the new biosensor was then used to monitor the activity of norbelladine 4-O'Methyltransferase (Nb4OMT) from the daffodil Narcissus pseudonarcissus in Escherichia coli. A structure-based self-supervised 3D residual neural network (3DResNet) trained to generalize at protein: non-protein interfaces, and the evolved biosensor was used to screen a panel of deep learning-guided Nb4OMT designs. Functional variants of the Nb4OMT enzyme were rapidly identified that yielded a 60% improvement in product titer, 2-fold higher catalytic activity, and 3-fold lower off-product formation.

### Results

### Identifying a Biosensor for the Branchpoint Amaryllidaceae Alkaloid 4-O'Methyl-Norbelladine

4-O'Me-norbelladine (4NB) is the branchpoint intermediate for the entire amaryllidaceae alkaloid (AA) family (FIG. 1A), and therefore was the target compound for biosensor generation. Previously the highly malleable TetR-family Salmonella typhimurium repressor RamR had been used as a starting point for identifying biosensors for a variety of benzylisoquinoline alkaloids (d′Oelsnitz, 2022). Given the chemical similarities between AAs and BIAs, and RamR's proven ability to rapidly evolve novel ligand specificity, RamR was again used as a starting point for directed evolution.

The wild-type RamR sensor was constitutively expressed on one plasmid (pReg-RamR) in parallel with another plasmid bearing the regulator's cognate promoter upstream of the sfGFP gene (Pramr-GFP). Upon induction with various AAs, RamR was found to be slightly responsive to both 4NB and its immediate precursor norbelladine, yielding 3.8 and 4.4-fold increases in fluorescence, respectively (FIG. 1B). To better understand this promiscuous binding activity, norbelladine was docked within the ligand binding pocket of RamR using GNINA 1.0 (Jumper, 2021), and a conformational pose was identified whereby the phenol moiety of norbelladine forms hydrogen bonds with S137 and T85, while the catechol moiety forms a hydrogen bond with K63. This docking pose also suggested that norbelladine's secondary amine may hydrogen bond with D152 and further interact with the aromatic ring of F155 (FIG. 1C).

### Evolving a Highly Specific Biosensor for 4-O'Methyl-Norbelladine

While the native responsiveness was promising, for practical use in metabolic engineering applications the sensitivity and specificity of RamR for 4NB needed to be greatly improved. The simulated molecular interactions between RamR and 4NB informed a rational approach to library design. Three site-saturated (NNS) RamR libraries that each targeted three residues facing inwards toward the ligand binding cavity were generated (FIG. 5). The 32,000 unique genotypes per library could be readily plumbed using our previously described method, Seamless Enrichment of Ligand Inducible Sensors (SELIS) (d′Oelsnitz, 2022). Briefly, this method involves a growth-based selection to first filter out biosensor variants that are incapable of repressing transcription from their cognate promoter, followed by a fluorescence-based screen to isolate sensor variants highly responsive to the target analyte.

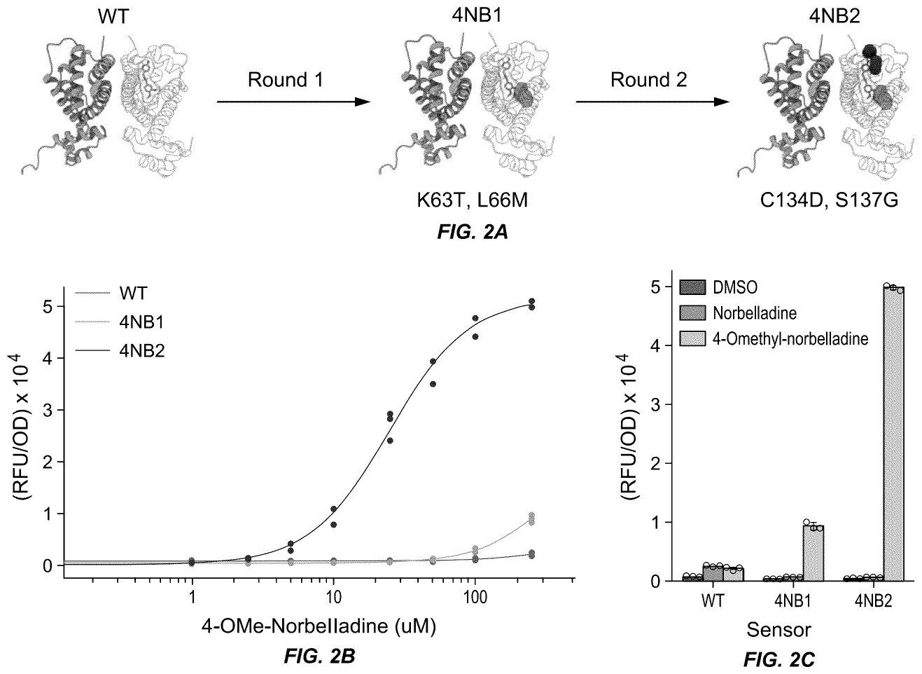

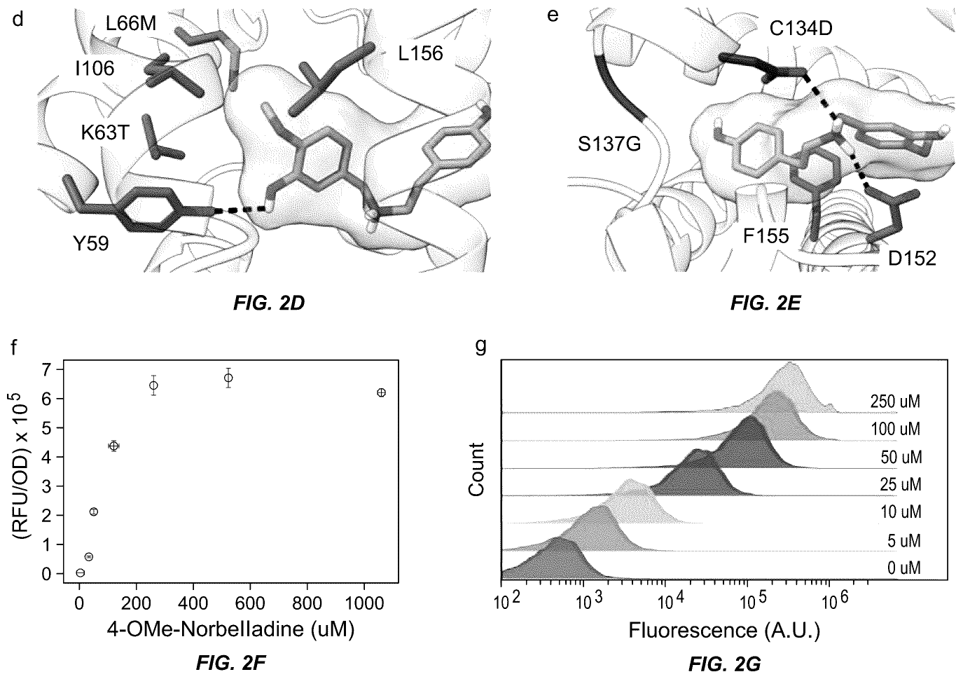

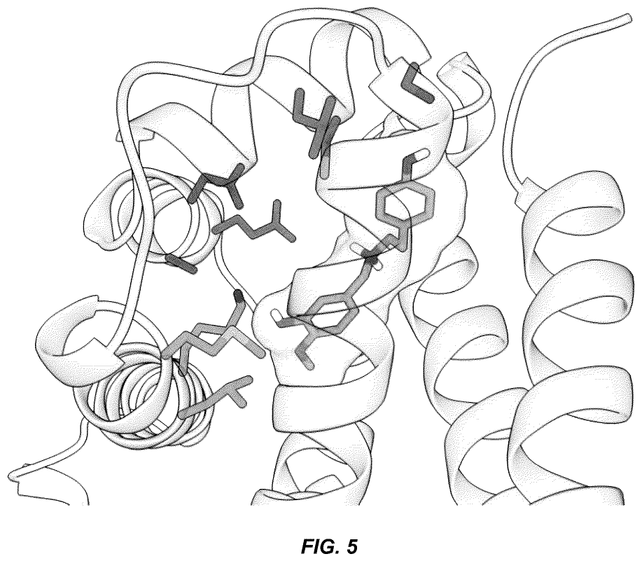

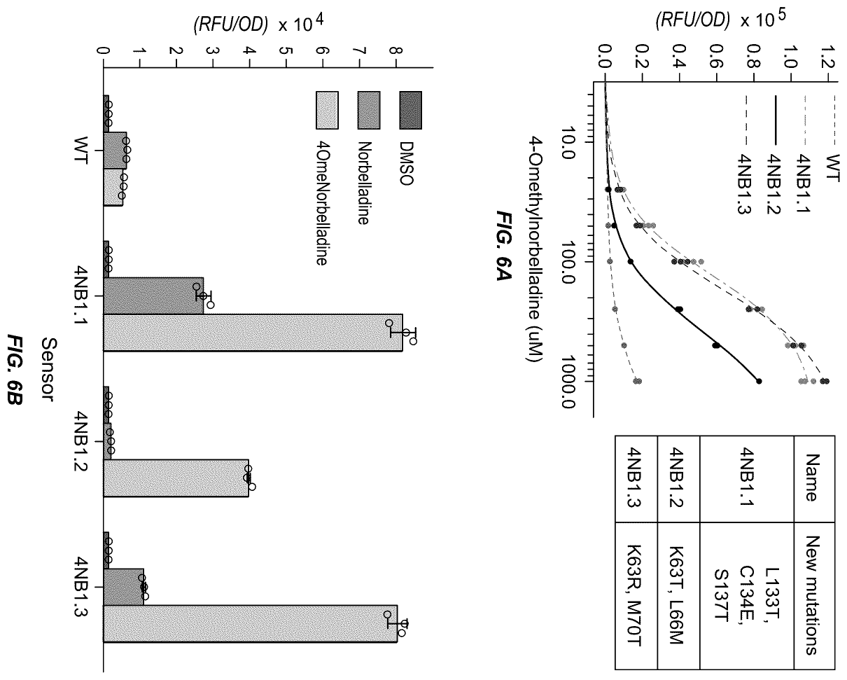

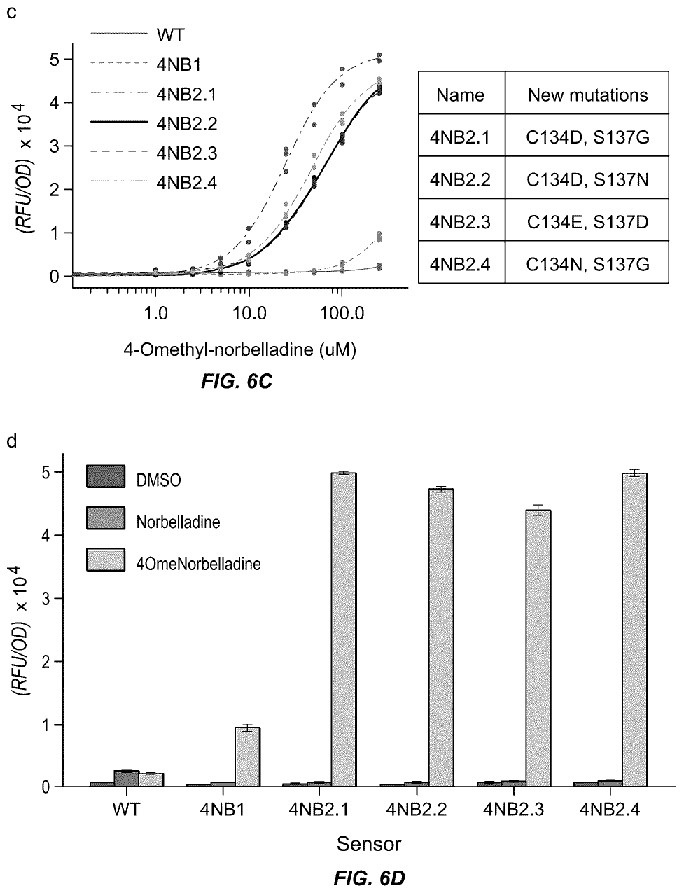

After the first round of directed evolution, several RamR variants were found to be substantially more responsive to 4NB, even in the absence of a negative selection against norbelladine. In fact, one variant bearing two amino acid substitutions (4NB1.2, K63T and L66M) displayed a 20-fold selectivity for 4NB over norbelladine (FIG. 6a, b). Using 4NB1.2 as a starting point, additional libraries were generated that encompassed the other, previously randomized positions. SELIS was now performed with a growth-based counter-selection against norbelladine (100 uM). The top four biosensor variants were again highly specific for 4NB but now also became significantly more sensitive, with the best variant, 4NB2.1 (C134D and S137G), achieving a limit of detection of approximately 2.5 uM (FIG. 6c, d; FIG. 2). Ultimately, the 4NB2.1 sensor was extremely selective for 4NB over norbelladine, displaying an over 80-fold preference for the former, despite the two effectors differing by only a single methyl group.

To again explore the structural basis for precise methyl group discrimination a structural model of 4NB2.1 was generated using AlphaFold 2.0 (Jumper, 2021), and 4NB was docked into this model using GNINA 1.0 (McNutt, 2021). The docked pose suggests that the K63T substitution repositions the hydroxyl group at position 3 of 4NB to hydrogen bond with the wild-type Y59 residue, while the L66M substitution strengthens a hydrophobic pocket around the 4-O'Methyl group of 4NB (along with the native 1106 and L156 residues; FIG. 2d). This analysis is in agreement with the fluorescence assay data, since only RamR variants bearing the K63T and L66M mutations are highly selective for 4NB over norbelladine (FIG. 6). The model also positions the new aspartate at position 134 (C134D) to hydrogen bond with the amine of 4NB; several other RamR variants also placed a hydrogen bond donor (glutamate, glutamine, asparagine) at the 134 position (FIG. 6). Overall, as a consequence of these substitutions the 4NB ligand may shift in position to allow for more favorable pi-pi stacking with F155 (FIG. 2c).

To evaluate the utility of the 4NB2.1 sensor for high-throughput screening of AAs, its performance was compared to an HPLC method adapted from the literature (Kilgore, 2014). The concentration range of 4NB can be discerned between 2.5 uM and 250 uM, while the equivalent range for the HPLC method is between 25 uM and 1000 uM (FIG. 2f). The dynamic range of sensing could potentially be further increased via less sensitive biosensor intermediates characterized during evolution (see FIG. 6). Most importantly, the 4NB2.1 sensor is approximately 10-fold more sensitive than the HPLC method, making it well-suited for screening transplanted biosynthetic enzymes from plants, which often initially show low flux (Cravens, 2019). Flow cytometry analysis indicated that the sensor's response at the population level was highly uniform (FIG. 2g), ensuring low noise measurements.

Monitoring Norbelladine O-Methyltransferase Activity in Escherichia coli

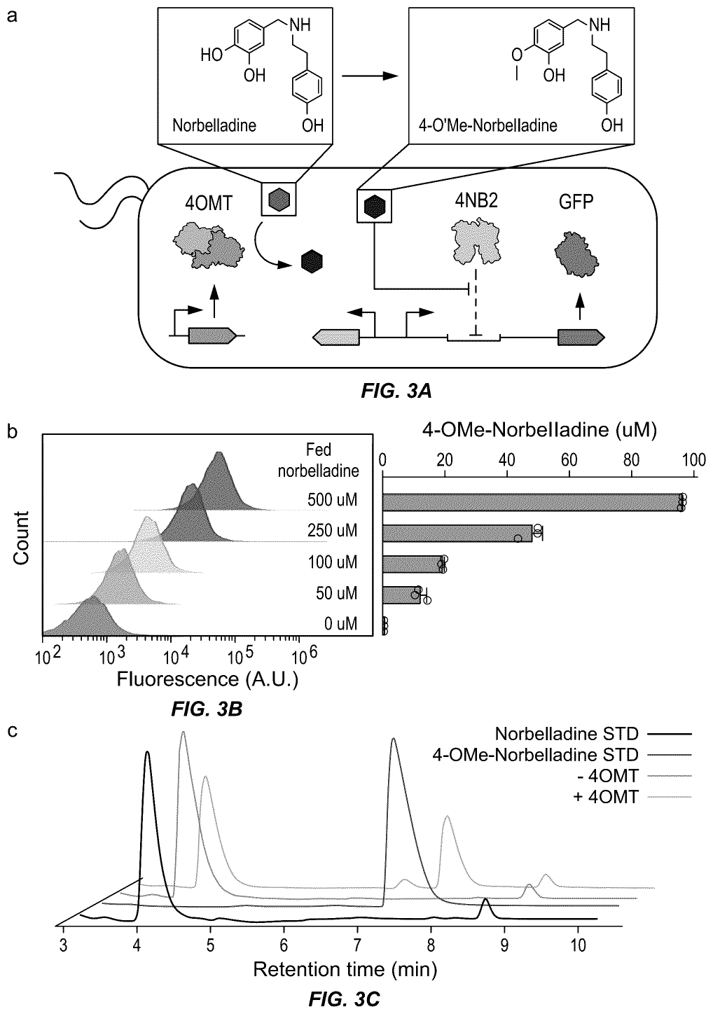

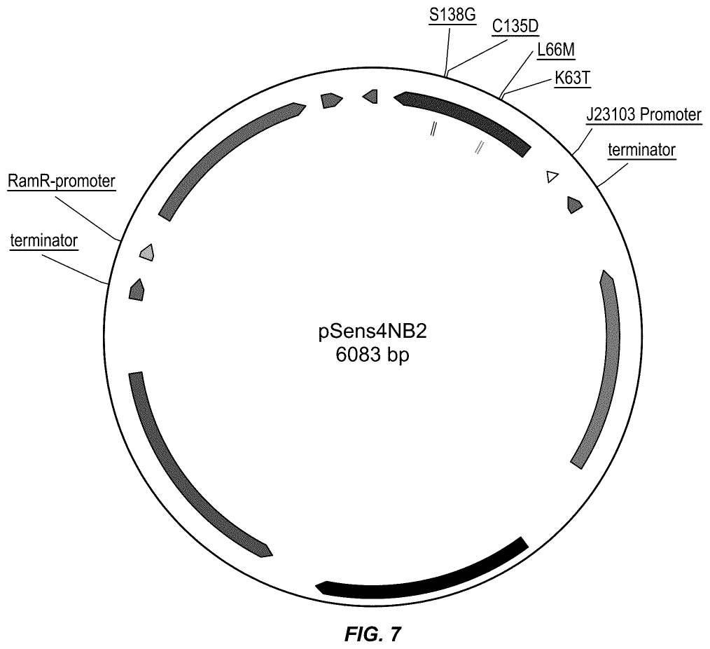

Although several AAs have been recognized for their therapeutic value, there have so far been no attempts to reconstitute AA pathways in microbial hosts. Since norbelladine 4-O-methyltransferase (Nb4OMT) from the wild daffodil Narcissus sp. aff. pseudonarcissus, is directly responsible for 4NB production from norbelladine, this was chosen as a starting point for development of a fuller pathway. A 4NB reporter plasmid (pSens4NB2; FIG. 7) was co-transformed with a plasmid constitutively expressing Nb4OMT. When this strain was grown in media supplemented with the substrate norbelladine, Nb4OMT activity could be observed, monitored, and quantified via fluorescence (FIG. 3a). The level of cell fluorescence correlated positively with enzyme expression strength (FIG. 8), with the concentration of norbelladine supplemented into the culture media, and with 4NB titer measured via HPLC (FIG. 3b). As was the case with measuring 4NB supplemented media, the fluorescence of cellular populations was uniformly distributed, again indicating that there was little noise during production or sensing. The independent measurements of noise via the 4NB biosensor will likely prove important as high yield strains are further developed and translated.

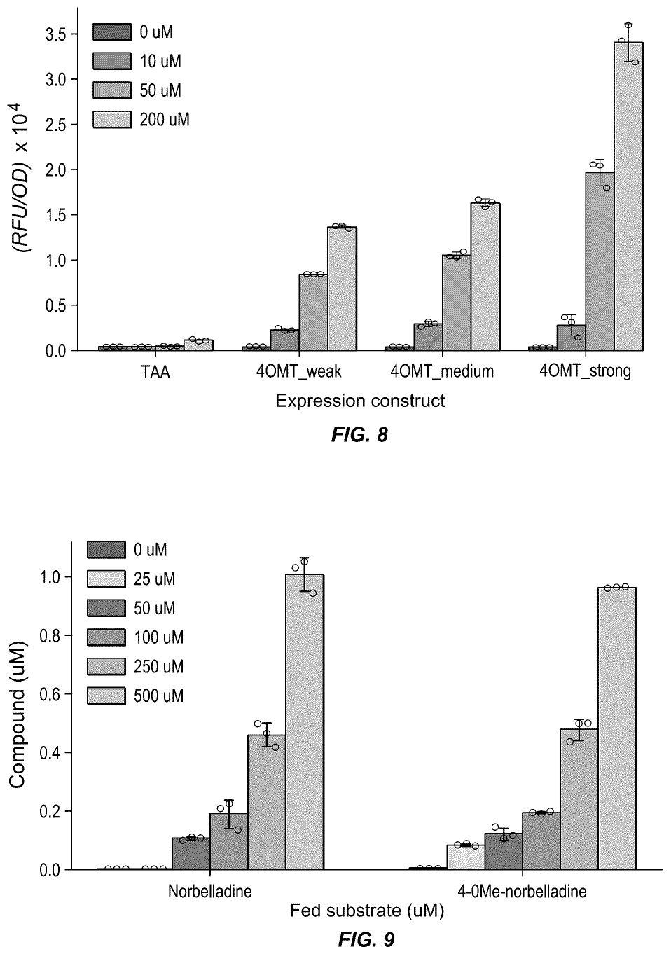

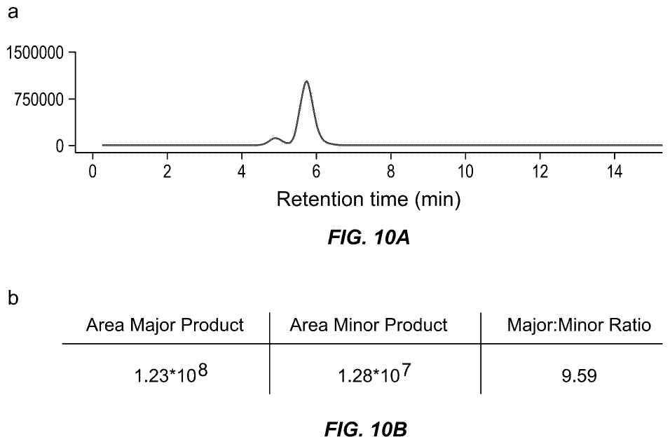

While these results demonstrated the utility of the evolved biosensor for monitoring Nb4OMT activity, they also revealed the catalytic inefficiency of the enzyme. HPLC analysis indicated that a significant amount of supplemented norbelladine remained after culturing for 24 hours (FIG. 3c). Indeed, leftover norbelladine was identified when as low as 50 uM of norbelladine was supplemented into the culture media (FIG. 9). Furthermore, LC/MS analysis identified 3-OMe-norbelladine as a minor component, indicating that the wild-type Nb4OMT enzyme was not highly regiospecific (FIG. 10). These observations all showed that Nb4OMT activity and specificity can be improved by enzyme engineering.

### Combining Custom Biosensors and Machine Learning to Improve Pathway Activity

To improve Nb4OMT activity in a microbial host directed evolution was carried out starting from randomly mutagenized libraries, via error-prone PCR, which generated an average of the three mutations per gene. The library of enzyme variants was transformed into cells containing the pSens4NB2.1 plasmid, plated on solid media containing norbelladine, and highly-fluorescent colonies were isolated and then individually phenotyped in a secondary liquid-based fluorescence screen. Interestingly, while this approach had previously proven effective for identifying improved enzyme variants in other pathways (d′Oelsnitz, 2022), it failed to enhance Nb4OMT activity.

A complementary approach to enzyme engineering was therefore pursued, using machine learning to better identify variants and potential library designs. A structure-based convolutional neural network (CNN; MutCompute) had previously proven adept at predicting mutations that improved protein functionalities, including fluorescence (BFP) (Shroff, 2020), expression (PMI) (Shroff, 2020), stability (polymerase) (Paik, 2023), and catalytic activity (PETase) (Lu, 2022). Unfortunately, the structure of the Nb4OMT enzyme had not been solved, preventing the generation of structure-based CNN predictions for substitutions. Instead, a de novo structural model for Nb4OMT was generated using Alphafold2 (Jumper, 2021), and both the S-adenosyl-homocysteine (SAH) cofactor and norbelladine were docked using GNINA1.0 (McNutt, 2021). The SAH cofactor was chosen instead of SAM because the nearest structure, of Alfalfa caffeoyl coenzyme A 30-methyltransferase (PDB: 1SUI; sequence similarity: 60.79%), contained this cofactor, and its SAH pose was transplanted to the AlphaFolded Nb4OMT scaffold. GNINA scored the minimized SAH pose with a 0.835 probability of being within 2 Å RMSD from the real pose and predicted an affinity of −7.9 kcal/mol (Table 1). The GNINA pose was guided by the supposition that either D155 or K158 must be the general-base that deprotonates the 4-hydroxyl group during the SN2 reaction, and that a potential cation-pi interaction with K158 would orient the plane of the catechol ring in the active site. GNINA scored the minimized norbelladine pose with a 0.824 probability of being within 2 Å RMSD from the real pose and a predicted affinity of −7.3 kcal/mol (Table 1).

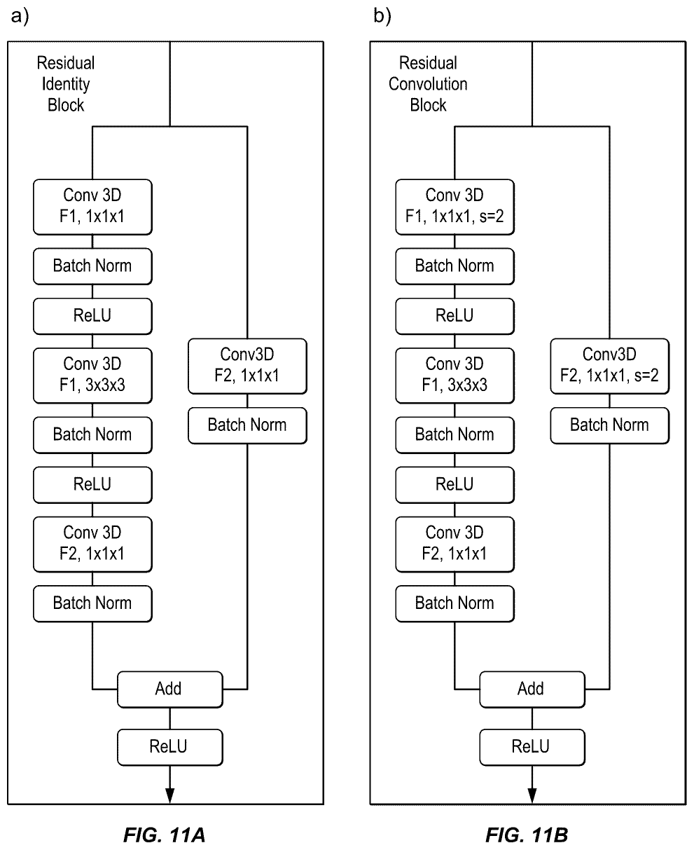

The original data engineering pipelines established for MutCompute restricted its training to microenvironments with atoms belonging to the 20 amino acids, and therefore MutCompute was unable to provide contextualized predictions in microenvironments that possessed atoms from cofactors or ligands (Shroff, 2020; Kulikova, 2021). To address this, the following took place: 1) rebuilt the data engineering pipelines to enable training on heterogenous microenvironments (see Methods), 2) curated new training and testing datasets that prioritized sampling these heterogeneous microenvironments (see Methods), and 3) developed a novel residual convolutional architecture to improve feature extraction capabilities and in turn the predictive power of the model (FIG. 11). The new self-supervised 3D residual neural network (3DResNet) achieved improved wildtype prediction accuracy ˜80% on a ˜250K residue test set compared to 69% on a 6K test set from the previous 3DCNN model (Shroff, 2020; Kulikova, 2021). Furthermore, the 3DResNets were shown to generalize to protein-ligand interaction interfaces without any drop in wildtype accuracy (81% wildtype accuracy on a protein-ligand interface test set compared to 62.1% from the previous 3DCNN model). After training numerous models, models for ensembling and ml-engineering of norbelladine methlytransferase were selected based on their zero-shot capability to correlate with ATM point mutations from FireProtDB (He, 2015) (zero-shot correlation described in methods). The ensembled 3DresNet model had an overall wildtype accuracy of 67.3% and protein-ligand interface wildtype accuracy of 66. %. The ternary computational Nb4OMT docked complex was passed to the improved 3DResNet model, and predictions were generated for each amino acid throughout the Nb4OMT protein. Based on these predictions, predicted substitutions were manually curated, prioritizing those that were near the active site and that were likely to form known stabilizing motifs such as salt bridges.

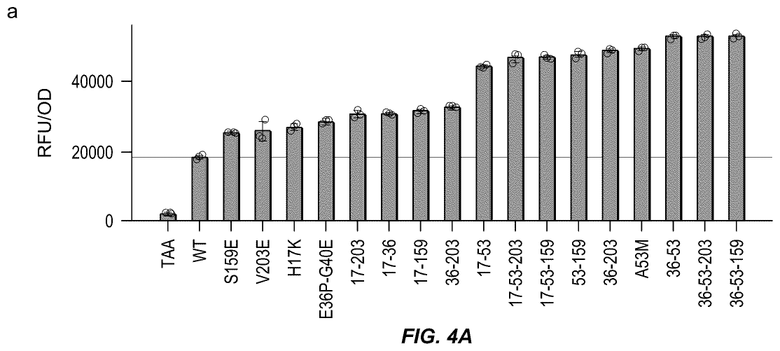

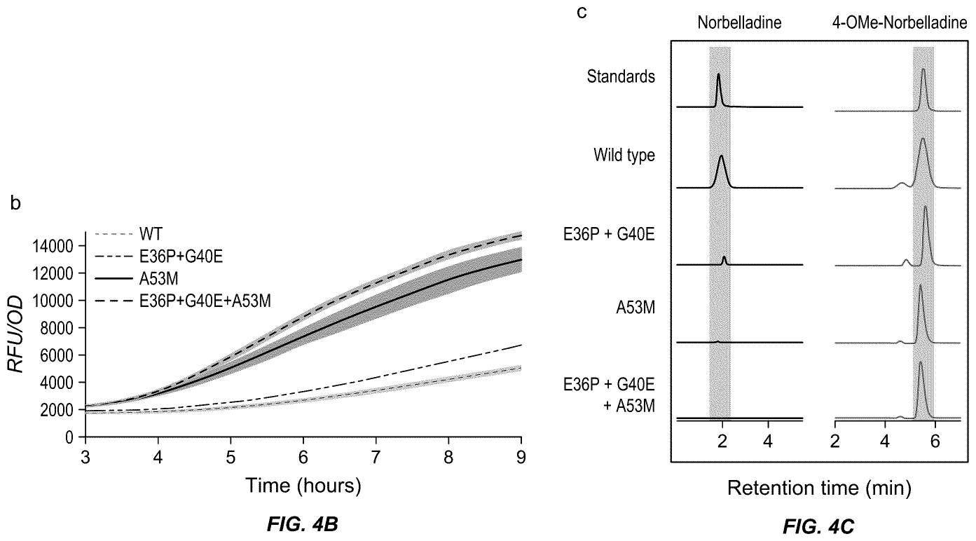

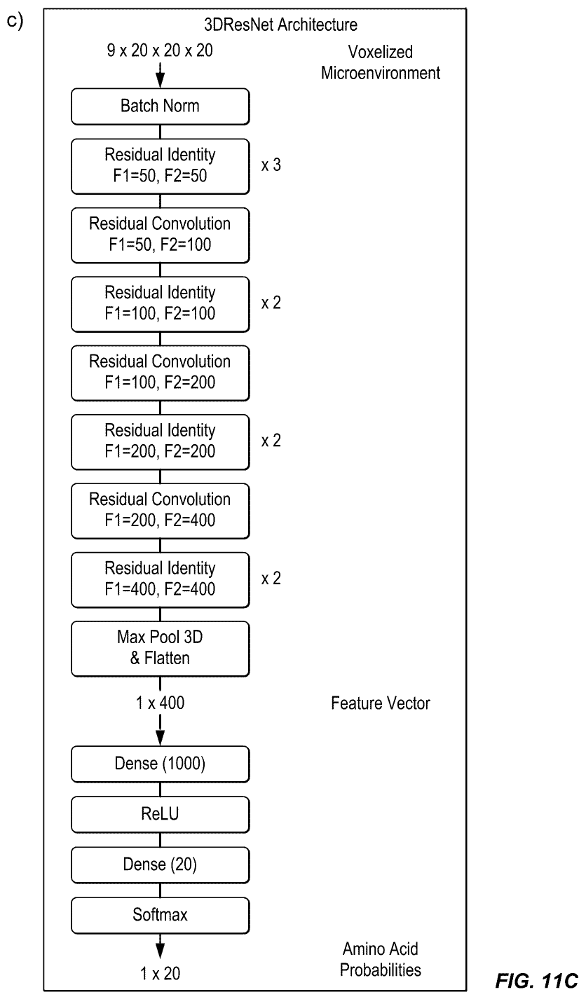

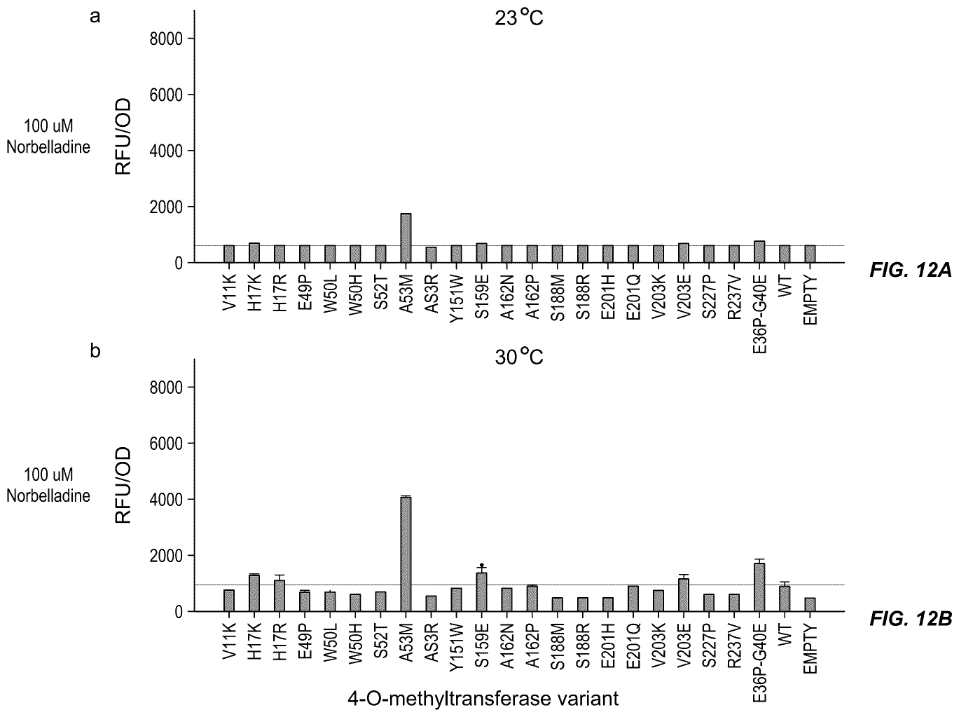

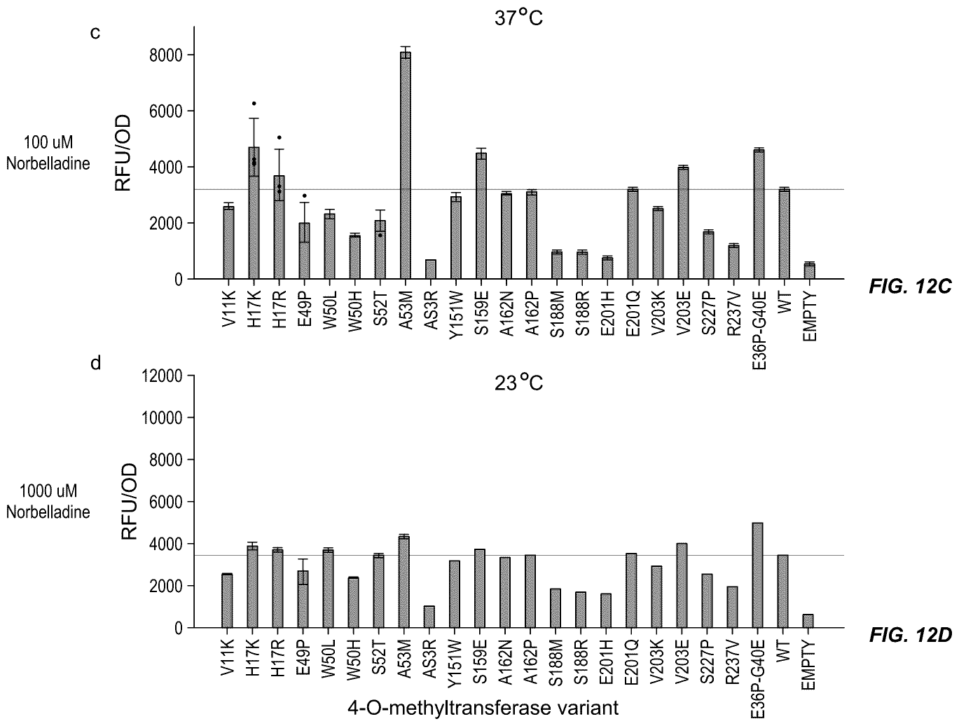

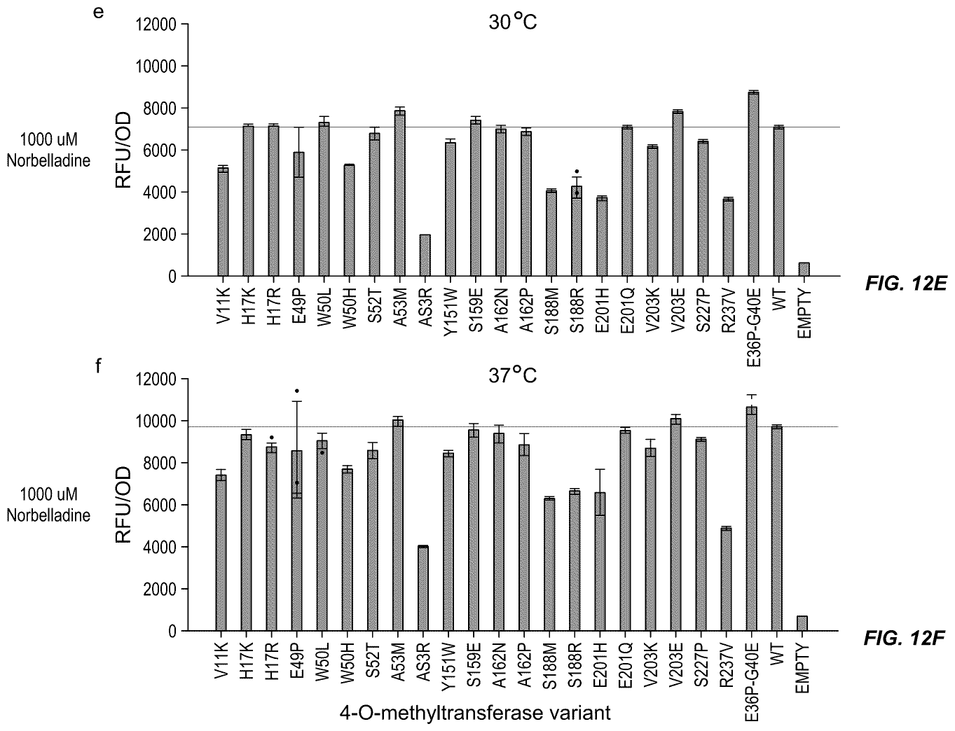

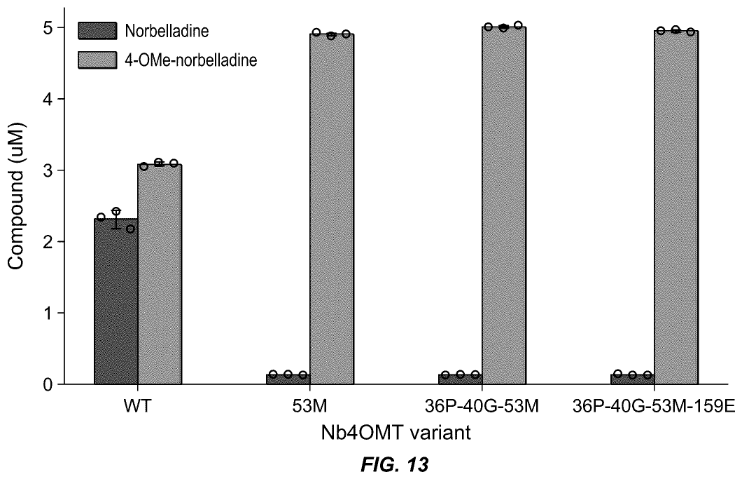

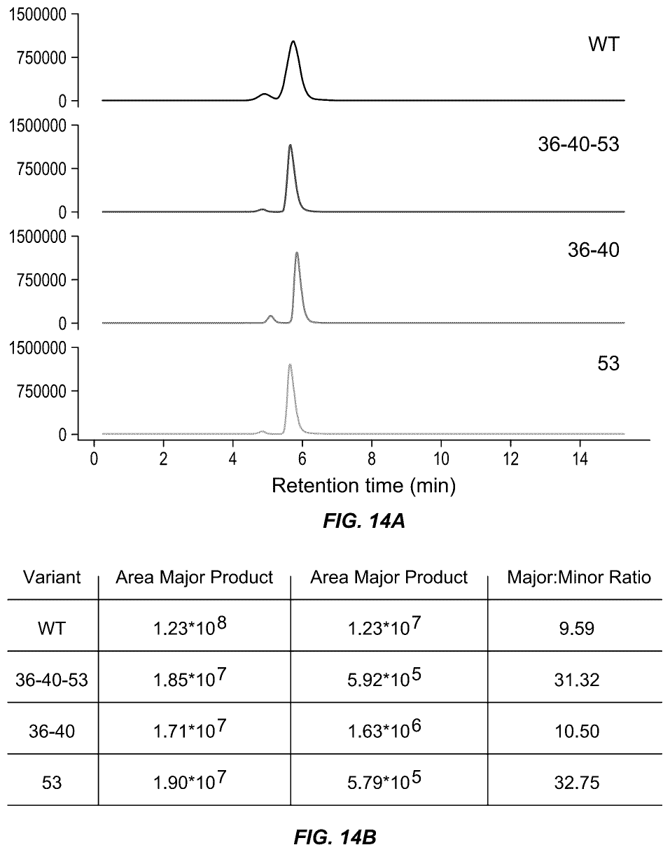

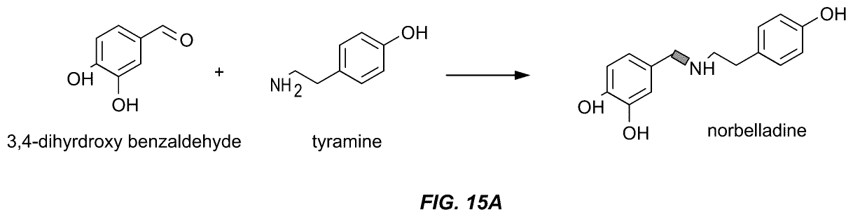

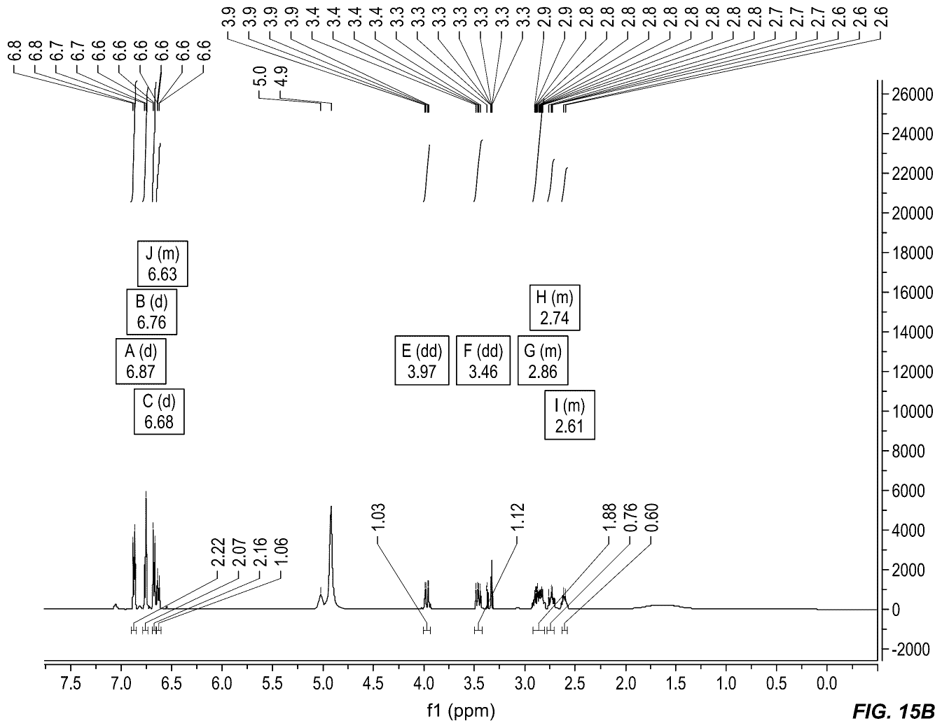

Ultimately, 22 mutational designs were experimentally validated in E. coli. Leveraging the biosensor-enabled high-throughput screen, each of the 22 mutants were quickly assessed across three temperatures (25° C., 30° C., 37° C.) and two substrate concentrations (100 uM, 1 mM). In all tested conditions, the A53M mutation consistently produced a fluorescent signal significantly above the wild-type enzyme, while the H17K, H17R, S159E, V203E, and E36P-G40E substitutions produced signals above wild-type in at least one tested condition (FIG. 12). Increasing the reaction temperature to 37° C. improved product formation (despite the fact that the Narcissus pseudonarcissus plant grows in 10-23° C. climates (He, 2016)). Double and triple mutants incorporating the H17K, A53M, S159E, V203E, and E36P-G40E substitutions were generated and screened; as with the initial screens, variants bearing the A53M mutation produced the greatest signals (FIG. 4A. In time course reactions, after media supplementation with norbelladine the rate of fluorescence increase for the E36P-G40E variant was similar to that of the wild-type enzyme, but the rates produced by the two A53M-bearing variants were significantly higher (FIG. 4B). LC/MS analyses were carried out on supernatants from the E36P-G40E, A53M, and E36P-G40E-A53M variants, and in agreement with our fluorescence-based assay the level of 4NB product increased by 60% while the level of remnant norbelladine decreased 17-fold (FIG. 4C, FIG. 13). The A53M mutation reduced levels of the 3-O'Methyl-norbelladine off-product by about 3-fold (FIG. 14).

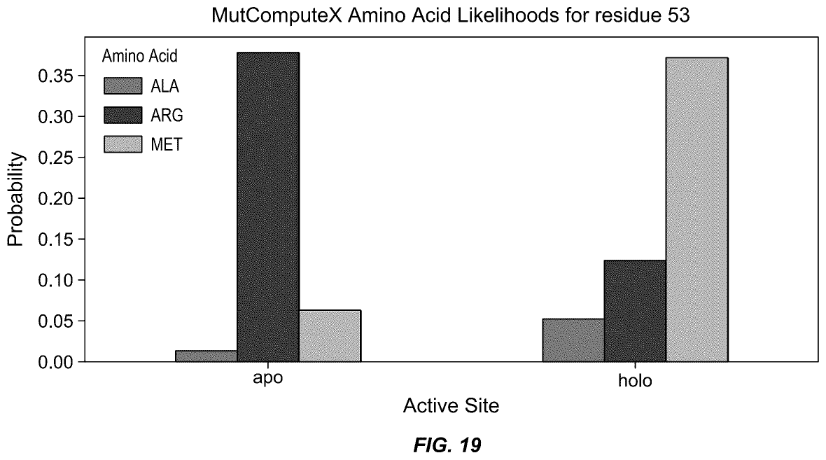

The beneficial A53M substitution was predicted by MutComputeX when the Nb40MT structure model was docked with SAH and norbelladine; in contrast, A53R was predicted when docking was not performed, a substitution that reduced activity under all tested conditions (FIGS. 12A-12F; FIG. 19). These results clearly demonstrate that the incorporation of ligand atoms to the microenvironment greatly improves MutComputeX's ability to engineer the active site of enzymes.

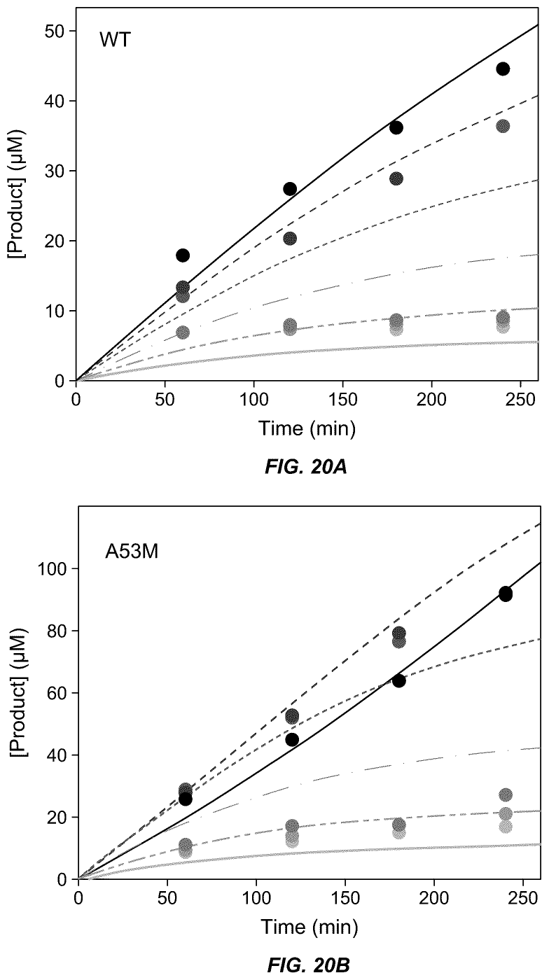

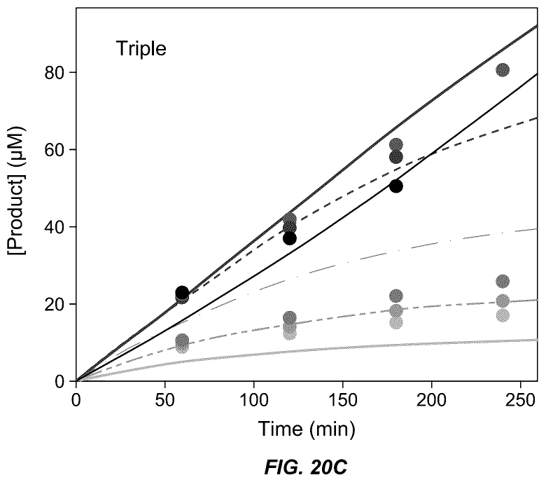

To further understand the mechanism behind beneficial mutations, the steady state kinetic and thermal properties of NbOMT bearing the A53M substitution alone or in combination with the E36P and G40E substitutions were characterized. To further understand the mechanism behind beneficial mutations, we characterized the steady state kinetic and thermal properties of NbOMT bearing the A53M substitution alone or in combination with the E36P and G40E substitutions. The A53M substitution increased kcal/Kₘ by a factor of about 2, due to a >2.1-fold increase in keut, and increased the Tₘ by 1.7° C. relative to the wild-type enzyme (Table 7; FIGS. 20A-20C). The Nb4OMT^{E36P/G40E/A53M} triple substitutions appeared to have keut and Kₘ values similar to the Nb4OMT^{A53M} single mutant, but a 5.6° C. increase in Tₘ relative to the wild-type Nb4OMT enzyme. Steady state kinetic data suggested that the Nb4OMT^{A53M} and Nb4OMT^{E36P/G40E/A53M} mutant enzymes were affected by substrate inhibition (FIGS. 20A-20C). These in vitro characterization data agree with the in vivo data collected with the 4NB-responsive biosensor (FIG. 4A).

### Crystal Structure of an Improved Norbelladine Methyltransferase

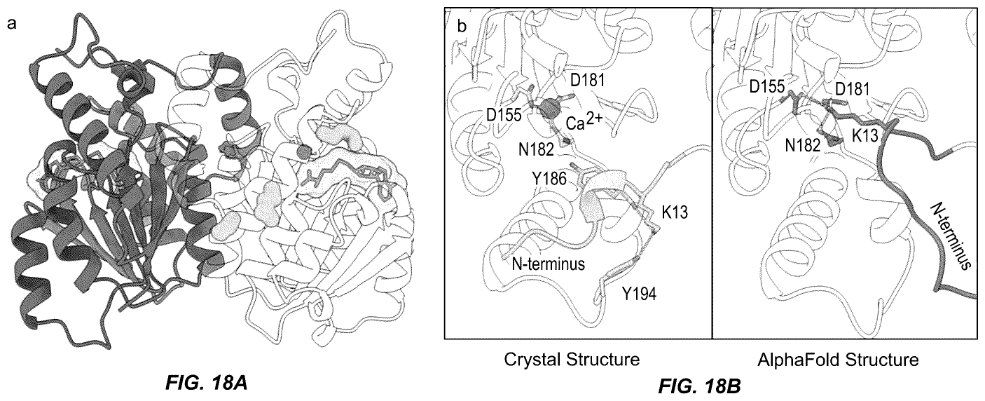

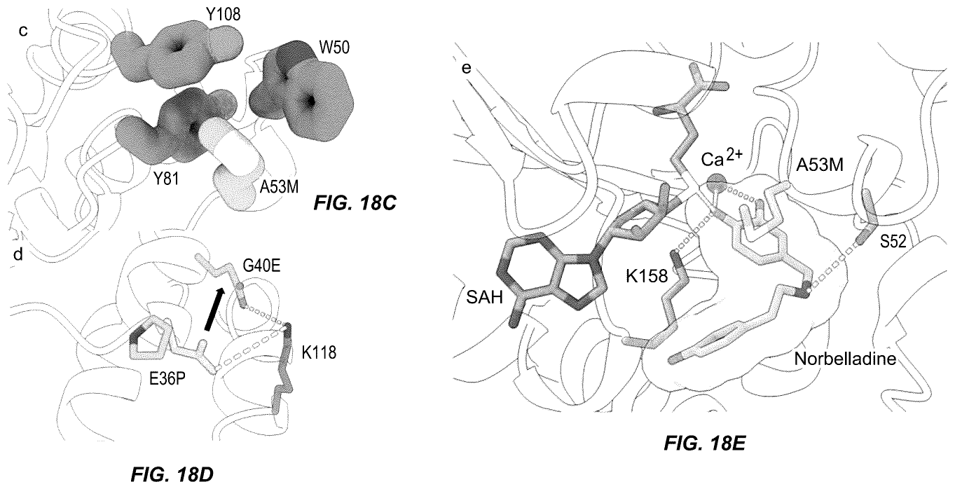

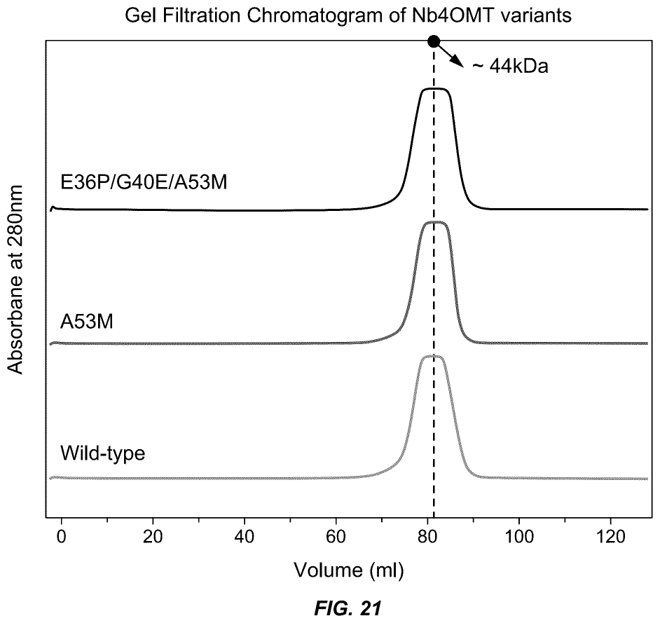

To better understand the mechanism underlying the three beneficial substitutions in the Nb4OMT^{E36P/G40E/A53M} variant, the structure of the Nb4OMT^{E36P/G40E/A53M} variant in complex with S-adenosyl-L-homocysteine (SAH) at 2.4 A resolution was determined. The Nb40MT variant exists as a homodimer in the crystalline form (FIG. 18A), consistent with its size exclusion chromatogram (FIG. 21). The overall fold of the protein was almost identical to the predicted AlphaFold2 structure, except for the N-terminal region (FIG. 18B). AlphaFold2 predicts that Lys13 forms tight salt bridge interaction with Asp155, Asp181, and Asn182 in the enzyme active site, while the experimental structure showed that Asp155, Asp181, and Asn182 instead coordinate a Ca²⁺ ion and Lys13 forms hydrogen bonds with the backbone of Tyr186 and the sidechain of Tyr 194 (FIG. 18B).

The experimental structure of Nb4OMT^{E36P/G40E/A53M} provided a basis for the improved thermostability of the enzyme (an increase in Tₘ from 52.8° C. to 58.4° C.). The A53M substitution inserted a larger hydrophobic methionine inside the hydrophobic pocket formed by Trp50, Tyr81, and Tyr108 (FIG. 18C), stabilizing the active site of Nb4OMT. The E36P-G40E double mutant shifted a glutamate from position 36 to position 40 and thereby preserved the salt bridge interaction with Lys118 while proline capping the alpha helix (FIG. 18D).

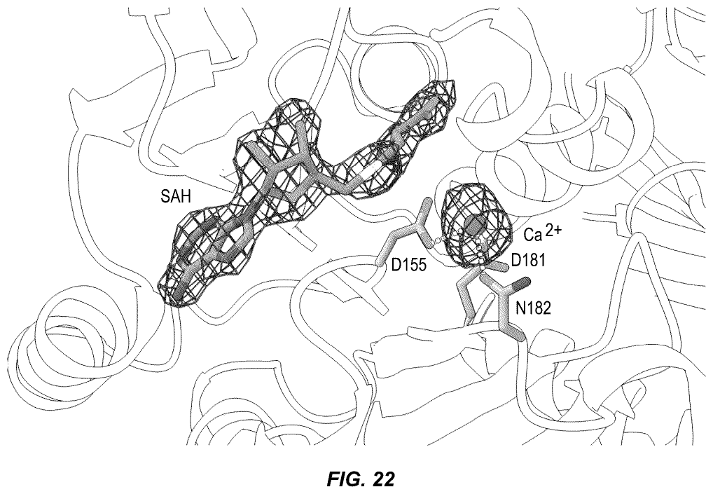

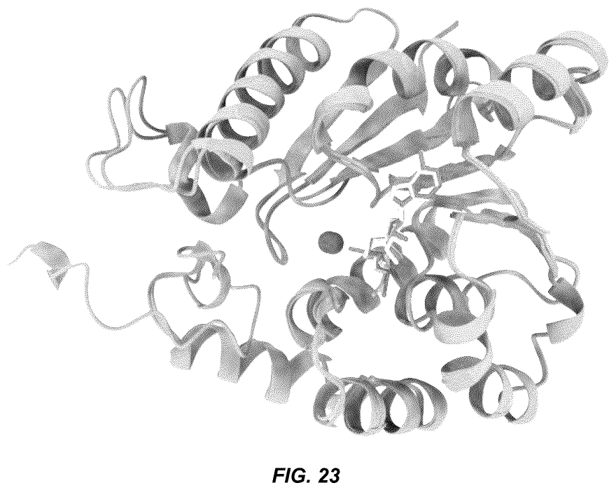

To better determine how the A53M substitution affects the substrate recognition of Nb4OMT, GNINA 1.0 was used to dock norbelladine into the crystal structure of Nb4OMT^{E36P/G40E/A53M} with SAH and Ca²⁺ already in the active site (based on Fo-Fc electron densities; FIG. 22). In the docked structure, the Ca²⁺ ion positions the catechol moiety of the substrate adjacent to the SAH binding site (FIG. 18E). A similar substrate recruitment by divalent metal ions was found in other, homologous methyltransferases (Ferrer, 2005; Jin, 2023). A sulfur-π interaction between the catechol group of norbelladine and Methionine 53 may also restrict the rotation of the catechol group, thereby reducing the cross-methylation of the 3′ position and improving specificity.

### DISCUSSION

Herein the use of directed evolution and machine learning-guided design for the development of custom microbial biosensors that could be used to monitor substantive improvements in amaryllidaceae alkaloid pathway activity are reported. The RamR transcription factor was evolved to respond to low micromolar levels of the pathway branchpoint 4NB, and after only four substitutions exquisite specificity emerges for the methylated oxygen moiety in 4NB, with a barely detectable response to the non-methylated precursor norbelladine. Overall, these results highlight the powerful capability of using evolved biosensors for precisely reporting on pathway intermediates while avoiding cross-reactivity with closely related precursor molecules. The RamR protein is now well positioned as an ideal starting point for the generation of biosensors for not only benzylisoquinoline alkaloids, but also for AAs such as galantamine, hacmanthaminc, lycorine, and their intermediates.

The high specificity was also used for measuring the real-time activity of the plant-derived Nb4OMT enzyme in E. coli, which in turn allowed for leveraging of the state-of-the-art 3DResNet, MutComputeX for enzyme engineering. Unlike structure prediction models (such as AlphaFold2 (Jumper, 2021), RosettaFold (Back, 2021), ESMfold (Lin, 2023), and OmegaFold (Wu, 2022)), or structure-based generative models (such as Rfdiffusion (Watson, 2023) and Ig-VAE (Eguchi, 2022)), MutcomputeX is a structure-based model designed to assess sequence substitutions, and that has been explicitly trained to generalize to non-protein atoms, such as nucleic acids and ligands. By leveraging recent developments in structure prediction (AlphaFold2) and ligand docking (GNINA1.0), a solved crystal structure is not needed to generate activity-enriched enzyme designs. MutComputeX was trained on ˜2.3M microenvironments sampled from over 23,000 protein structures, and predicted functional variants of the Nb4OMT enzyme with 60% improvement in product titer, 17-fold reduced remnant substrate, and 3-fold lower off-product formation. Starting for the first time from an AlphaFold structure model docked with its substrate and cofactor, MutComputeX designs yielded variants with not only improved product: substrate ratios, but also improved regiospecificities, as determined by LC/MS analysis.

Synergizing custom biosensor-enabled screens with self-supervised machine learning-guided protein design can fundamentally accelerate the pace of strain and enzyme engineering as a whole. Custom biosensor-enabled screens enable rapid collection of phenotype data under a wide variety of experimental conditions, including determining the kinetics of product formation among strain and enzyme variants, values that are nearly impossible to measure using traditional analytical instruments. The importance of machine learning is further highlighted by failed attempts to engineer Nb4OMT using random mutagenesis alone. Microbial semi-synthesis of galantamine and other AAs can provide faster production cycles, a more reliable supply chain, and reduced land and water use compared to traditional plant harvesting methods, and the biosensor-AI hybrid technology stack which have been advanced herein can greatly accelerate the engineering of upstream enzymes in the pathway, such as norbelladine synthase and norcraugsodine reductase (Back, 2021; Wu, 2022).

### Methods

### Strains, Plasmids and Media

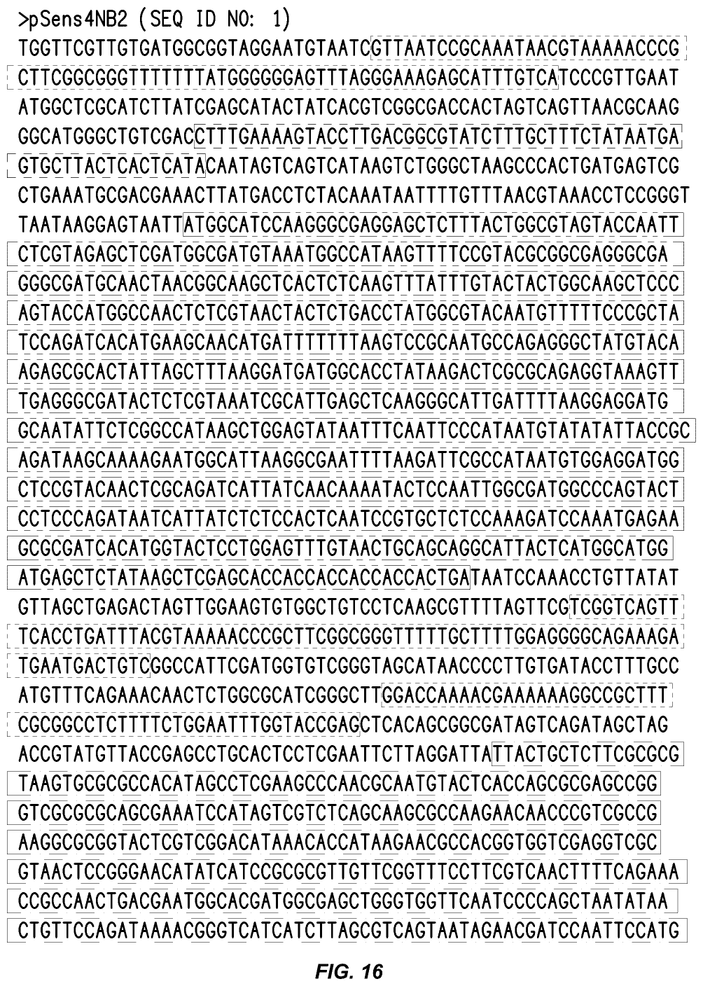

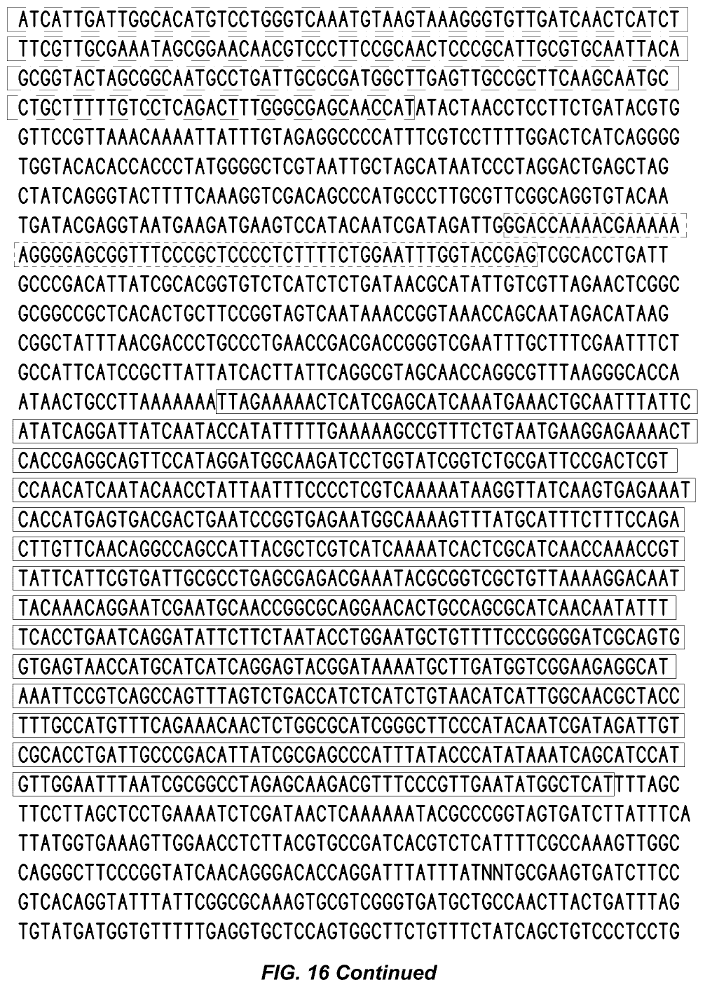

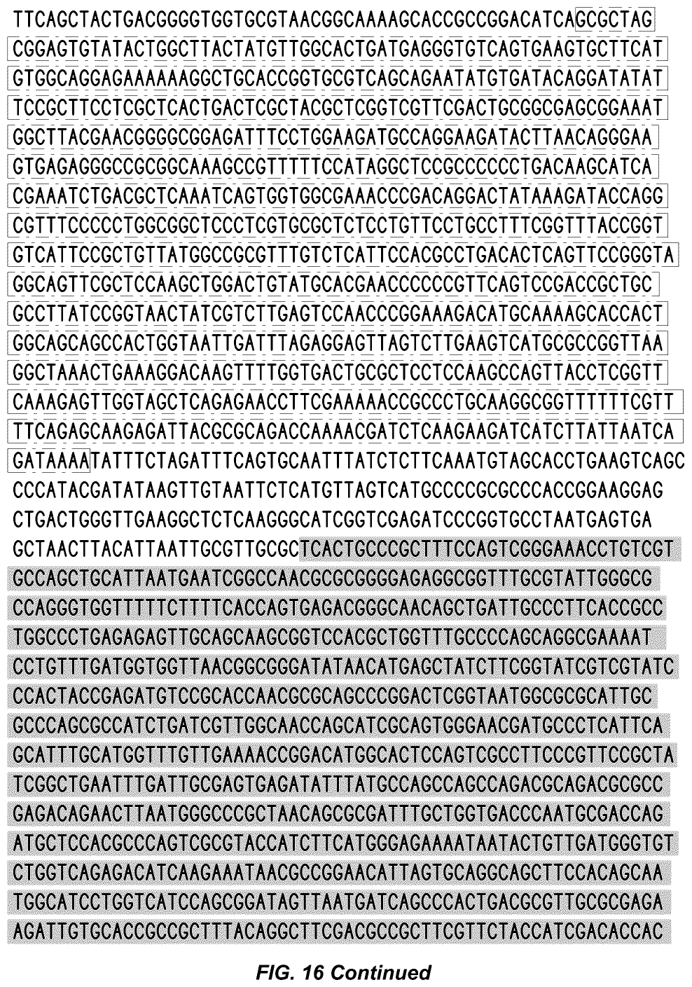

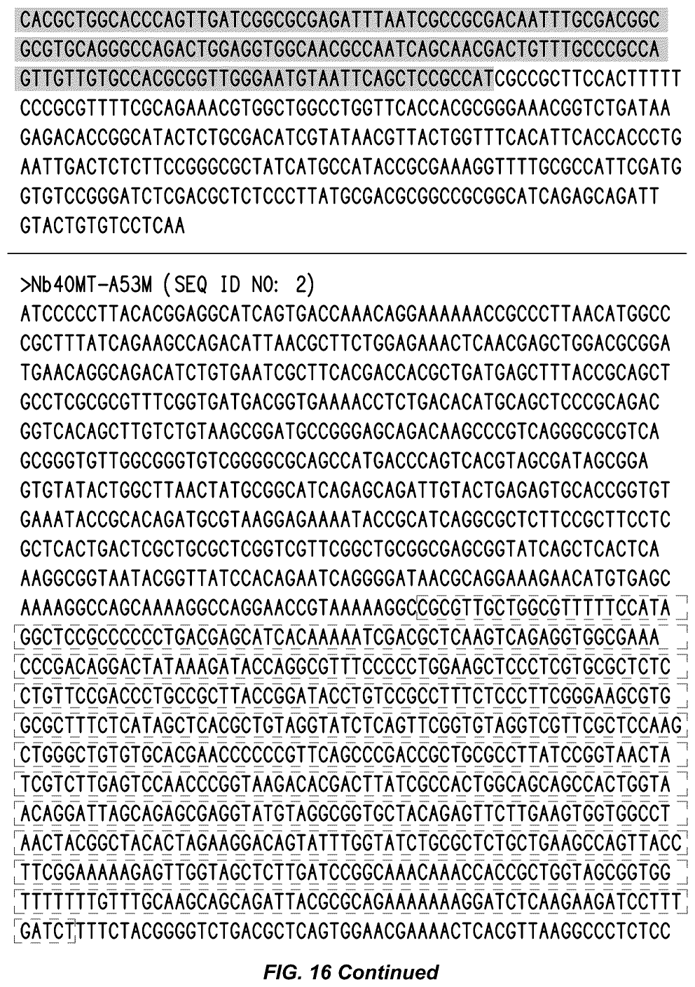

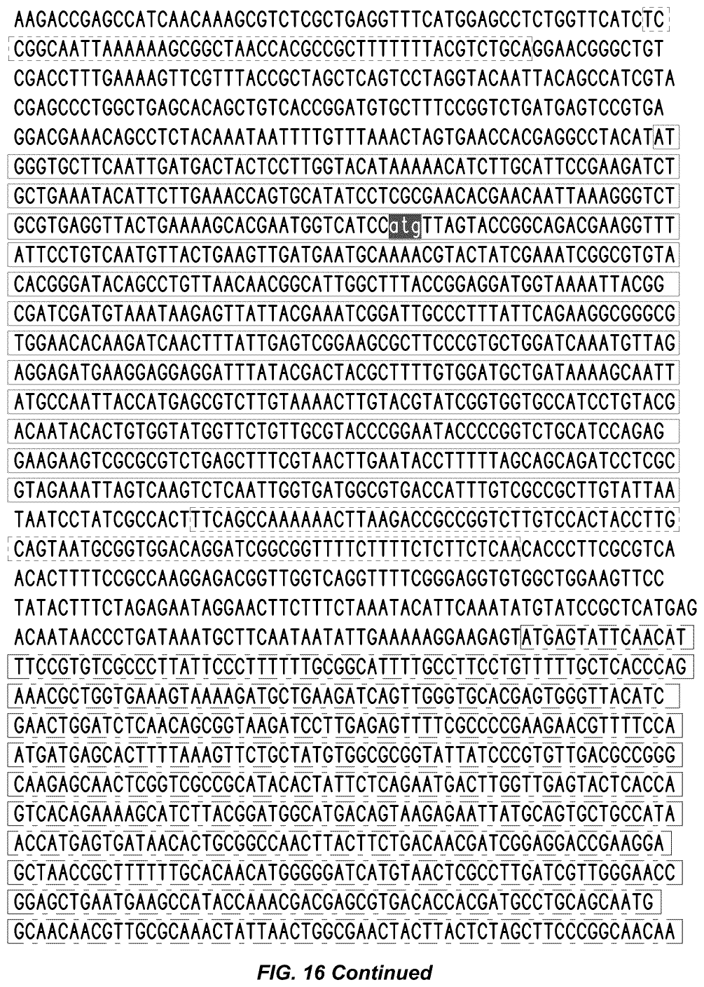

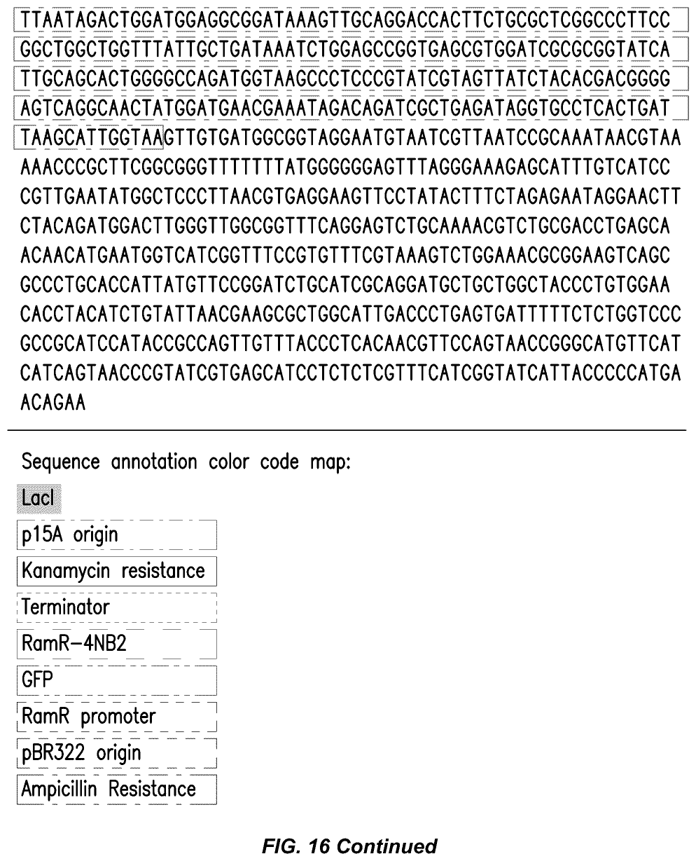

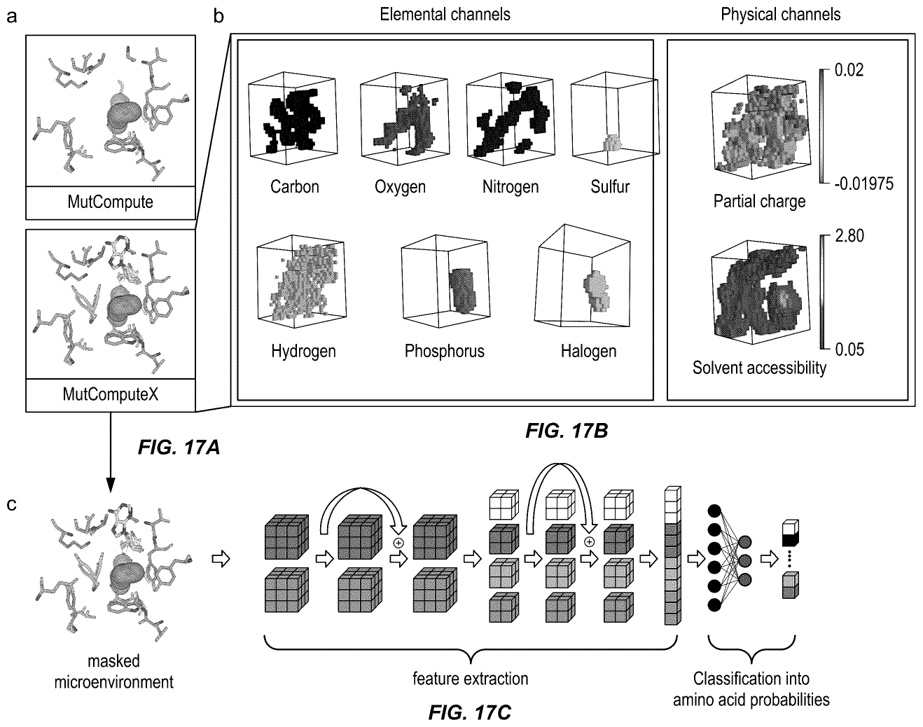

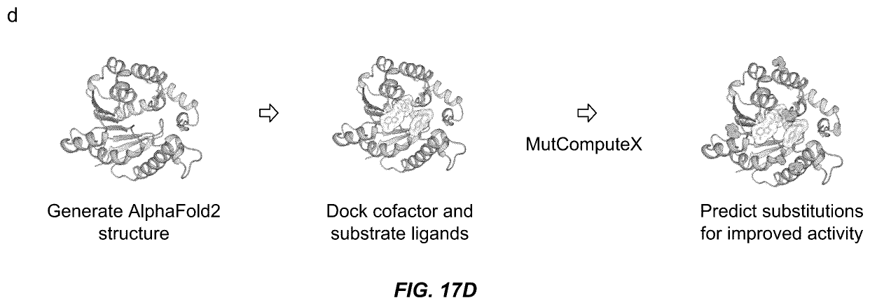

E. coli DH10B (New England Biolabs) was used for all routine cloning and directed evolution. All biosensor systems were characterized in E. coli DH10B. LB Miller (LB) medium (BD) was used for routine cloning, fluorescence assays, directed evolution and orthogonality assays unless specifically noted. LB with 1.5% agar (BD) plates were used for routine cloning and directed evolution. The plasmids described in this work were constructed using Gibson assembly and standard molecular biology techniques. Synthetic genes, obtained as gBlocks, and primers were purchased from IDT. Plasmid designs and sequences are listed in FIG. 16.

### Chemicals

4-O'Methylnorbelladine was purchased from Toronto Research Chemicals (Toronto Research Chemicals. CAT #: H948930). Tyramine (T90344), 3,4-dihydroxybenzaldehyde (37520), dichloromethane (439223), and NaBH₄ were purchased from Sigma Aldrich. NMR solvents (d⁶-DMSO, CD₃OD) were purchased from Cambridge isotope laboratories.

### Chemical Synthesis and NMR Analysis of Norbelladine

The aldehyde (3,4-dihydroxybenzaldehyde) (1 mM, 138 mg) and tyramine (1 mM, 137 mg) were dissolved in dichloromethane (5 mL) and converted to the imine in situ compound by stirring for 4 hr at room temperature. The imine compound was reduced with NaBH₄ (2 mM, 75.6 mg), washed with water and dried to produce crude product. The crude material was then purified by combinatorial flash chromatography to yield norbelladine (10-90% MeCN in H2O, 20 min; 130 mg recovered, beige orange solid, 50% yield), which was confirmed via NMR (FIGS. 14A-14B). NMR spectra were taken on the 500 MHZ Bruker prodigy at University of Texas at Austin.

### Chemical Transformation

For routine transformations, strains were made competent for chemical transformation. Five milliliters of an overnight culture of DH10B cells was subcultured into 500 mL LB medium and grown at 37° C. and 250 r.p.m. until an optical density of 0.7 was reached (˜3 h). Cultures were centrifuged (3,500 g, 4° C., 10 min), and pellets were washed with 70 mL chemical competence buffer (10% glycerol, 100 mM CaCl2) and centrifuged again (3,500 g, 4° C., 10 min). The resulting pellets were resuspended in 20 mL chemical competence buffer. After 30 min on ice, cells were divided into 250-μL aliquots and flash frozen in liquid nitrogen. Competent cells were stored at −80° C. until use.

### Biosensor Response Assay

The pReg-RamR and Pramr-GFP plasmids were co-transformed into DH10B cells, which were then plated on LB agar plates containing appropriate antibiotics. Three separate colonies were picked for each transformation and were grown overnight. The following day, 20 μL of each culture was then used to inoculate six separate wells in a 2-mL 96-deep-well plate (Corning, P-DW-20-C-S) sealed with an AeraSeal film (Excel Scientific) containing 900 μL LB medium, one for each test ligand and a solvent control. After 2 h of growth at 37° C., cultures were induced with 100 μL LB medium containing either 10 μL DMSO or 100 μL LB medium containing the target AA dissolved in 10 μL DMSO. Cultures were grown for an additional 4 h at 37° C. and 250 r.p.m. and subsequently centrifuged (3,500 g, 4° C., 10 min). Supernatant was removed, and cell pellets were resuspended in 1 mL PBS (137 mM NaCl, 2.7 mM KCl, 10 mM Na2HPO4, 1.8 mM KH2PO4, pH 7.4). One hundred microliters of the cell resuspension for each condition was transferred to a 96-well microtiter plate (Corning, 3904), from which the fluorescence (excitation, 485 nm; emission, 509 nm) and absorbance (600 nm) were measured using the Tecan Infinite M1000 plate reader.

### RamR Library Design and Construction

Three semi-rational libraries were designed, each targeting three inward-facing residues within the RamR ligand-binding pocket (FIG. 5). Libraries were generated using overlap PCR with redundant NNS codons using AccuPrime Pfx (Thermo Fisher, 12344024) and cloned into pReg-RamR. E. coli DH10B bearing pSELIS-RamR was transformed with the resulting library. Transformation efficiency always exceeded 10⁶ for each round of selection, indicating several fold coverage of the library. Transformed cells were grown in LB medium overnight at 37° C. with carbenicillin and chloramphenicol.

### Directed Evolution of RamR Biosensors

Cell culture (20 μl) bearing the sensor library was seeded into 5 ml fresh LB containing appropriate antibiotics, 100 μg ml-1 zeocin (Thermo Fisher, R25001) and 100 uM of norbelladine (for round two) and grown at 37° C. for 7 h. Following incubation, 0.5 μl of culture was diluted into 1 ml LB medium, from which 100 μl was further diluted into 900 μl LB medium. Three hundred microliters of this mixture was then plated across three LB agar plates (100 μL per plate) containing carbenicillin, chloramphenicol and 4NB dissolved in DMSO. Plates were incubated overnight at 37° C. The following day, the brightest colonies were picked and grown overnight in 1 ml LB medium containing appropriate antibiotics in a 96-deep-well plate sealed with an AeraSeal film at 37° C. A glycerol stock of cells containing pSELIS-RamR and pReg-RamR encoding the template RamR variant was also inoculated into 5 ml LB for overnight growth.

The following day, 20 μl of each culture was used to inoculate two separate wells in a new 96-deep-well plate containing 900 μl LB medium. Additionally, eight separate wells containing 1 ml LB medium were inoculated with 20 μl of the overnight culture expressing the parental RamR variant. After 2 h of growth at 37° C., the top half of the 96-well plate was induced with 100 μl LB medium containing 10 μl DMSO, whereas the bottom half of the plate was induced with 100 μl LB medium containing 4NB dissolved in 10 μl DMSO. The concentration of 4NB used for induction is typically the same concentration used in the LB agar plate for screening during that particular round of evolution. Cultures were grown for an additional 4 h at 37° C. and 250 r.p.m. and subsequently centrifuged (3,500 g, 4° C., 10 min). Supernatant was removed, and cell pellets were resuspended in 1 ml PBS. One hundred microliters of the cell resuspension for each condition was transferred to a 96-well microtiter plate, from which the fluorescence (excitation, 485 nm; emission, 509 nm) and absorbance (600 nm) were measured using the Tecan Infinite M1000 plate reader. Clones with the highest signal-to-noise ratio (generally the top 5-10% of the screened clones) were then sequenced and subcloned into a fresh pReg-RamR vector.

For sensor variant validation, the subcloned pReg-RamR vectors expressing the sensor variants were transformed into DH10B cells expressing Pramr-GFP. These cultures were then assayed, as described in Biosensor response assay, using eight different concentrations of the 4NB. The sensor variant that displayed a combination of low background, a reduced EC₅₀ for 4NB and a high signal-to-noise ratio was then used as the template for the next round of evolution.

### Dose-Response Measurements

Glycerol stocks (20% glycerol) of strains containing the plasmids of interest were inoculated into 1 ml LB medium and grown overnight at 37° C. Twenty microliters of overnight culture was seeded into 900 μl LB medium containing ampicillin and chloramphenicol in a 2-ml 96-deep-well plate sealed with an AeraSeal film. Following growth at 37° C. and 250 r.p.m. for 2 h, cultures were induced with 100 μl of an LB medium solution containing appropriate antibiotics and the inducer molecule dissolved in 10 μl DMSO. Cultures were grown for an additional 4 h at 37° C. and 250 r.p.m. and subsequently centrifuged (3,500 g, 4° C., 10 min). Supernatant was removed, and cell pellets were resuspended in 1 ml PBS. The cell resuspension (100 μl) for each condition was transferred to a 96-well microtiter plate, from which the fluorescence (excitation, 485 nm; emission, 509 nm) and absorbance (600 nm) were measured using the Tecan Infinite M1000 plate reader.

### Biosensor-Linked O-Methyltransferase Activity Assay

Nb4OMT was expressed with the P150-RBS (riboJ) promoter-RBS on the pReg-RamR plasmid backbone (no regulator present). Cells were co-transformed with both the 40MT plasmid and the 4NB reporter plasmid and plated on an LB agar plate containing appropriate antibiotics. Three individual colonies from each transformation were picked into LB and grown overnight. Resulting cultures were diluted 50-fold into 1 ml LB medium containing the indicated concentration of norbelladine in a 96-deep-well plate and were grown at the indicated temperature for 24 h. Subsequently, the fluorescence of cultures was measured in the same manner as previously described in Dose-response measurement above.

Ternary Complex Generation with AlphaFold2 and GNINA1.0

Nb4OMT wild type sequence (uniprot id: A0A077EWA5) was run through the AlphaFold2-multimer as a homodimer using the publicly available collab notebook. This resulted in a computational structure with a pLDDT of 0.955 and a pTM of 0.94. The initial coordinates for the SAH cofactor were transplanted onto the AlphaFold structure from the 1SUI pdb structure and then optimized with GNINA1.0's—local_only and—minimize flags. Norbelladine's initial 3D coordinates were obtained from the PubChem database (id: 416247) and docked into the active site of the A protomer. To dock norbelladine, we generated a bounding box for the GNINA docking procedure by finding the largest 3D box from the atomic coordinates of the following residues: L10, W50, S52, A53, D155, D157, K158, W185, Y186, A204. GNINA was run several times with different seeds and all docked poses were manually screened for known mechanistic insight. The docked pose that best satisfied the mechanistic insight and received a high GNINA docking score was then minimized with the—local_only and—minimize flags. The docking results from GNINA for SAH and NB are shown in Table 1.

### Building MutComputeX:

### Structure File Pre-Processing:

To generate voxelized matrices of microenvironments that span between protein: non-protein atoms, experimental CIF files were pre-processed with 1) ChimeraX to add hydrogen atoms to the proteins, nucleic acids, and organic ligands; 2) ChargeFW2 to add polarized charges that bridge protein: non-protein interfaces; and 3) FreeSASA to add solvent accessible surface area values that take into account protein: non-protein interactions. CIF read and write functionality for ChargeFW2 and FreeSASA were implemented and merged to both open-sourced libraries.

### Voxelized Matrix Generation:

To generate a voxelized molecular representation of a microenvironment, a 20 Å cube of atoms was filtered from the structure centered on the Calpha and oriented with respect to the backbone where the side chain was along the +z axis. All atoms in the center residue are then removed prior to insertion into a voxelized grid with 1 Å resolution. Each atom is placed into a corresponding element channel except halogen atoms (which are placed into a multi-atom channel that consist of F, Cl, Br, I) and each atom's partial charge and SASA value are placed into the partial charge and SASA channels, respectively. For all channels, atom values are gaussian blurred according to their Van-der-Waals radii. The P and Halogen channels were added to the original MutCompute framework in order to generalize to ligands and nucleic acids.

### Dataset Generation:

A dataset of 50% sequence similar protein chains with at least a 3.0 Å resolution was downloaded in November 2021 from the RCSB. This provided us with X protein sequences from Y PDB entries. To generate microenvironment datasets, each protein where residues within 5 Å of a non-protein entity were prioritized and then randomly backfilled until 200 residues or half of the protein sequence was sampled. A total of 2,569,256 microenvironments were sampled from 22,759 protein sequences and split 90:10 to generate our training and test set splits for interfaces and non-interface residues are shown in Table 2.

### Model Training:

The 3D residual neural network was built in Tensorflow 2.7. The architecture is provided in FIG. 11. Each model run was parallelized over 4 AMD Radeon Instinct MI50s with a batch size of 200. Models were trained for up to 8 epochs where each epoch was saved as a checkpoint with a variety of hyperparameters. A scheduled learning rate was used that began at 0.001 and had an exponential decay constant of either 0.3 or 0.5 and an adaptive learning rate that would lower the learning rate by 0.25 if the training accuracy did not improve by 0.1% after either 30K, 50K, and 60K training instances. Weights were updated with the Adam optimizer and all convolutional layers had weight decay regularization of 0.001.

### Model Benchmarking:

To ensure the datasets were enabling the 3D ResNet models to generalize across protein: non-protein interfaces, we monitored the overall wildtype accuracy and wildtype accuracy for residues at DNA, RNA, and ligand interfaces on our test set. To select models to ensemble and generate engineering predictions zero shot-predictions were engineered for all mutational data in FireProtDB and chose the models that had the highest correlation with the single point mutation ATM experimental data. The zero-shot predictions were generated by taking the prediction assigned to the wildtype and mutant amino acid from FireProtDB and taking the log odds where a positive log odd means a stabilizing prediction and a negative log odd means a destabilizing prediction. The ensembled model had a Pearson and Spearman correlation coefficients of 0.367 and 0.425 with the 2719 single point mutations with ATM experimental data in FireProtDB and a Pearson and Spearman correlation coefficients of −0.407 and −0.457 with the 4889 single point mutations with ΔΔG experimental data in FireProtDB. Correlation coefficients for the independent models can be found in Table 3.

### Mutational Designs

Mutations were designed with two goals: stabilizing the protein away from the active site and investigating point mutations where predictions differed between the docked and apo protein structures. With these objectives, residues were sorted based on the log odds between the predicted and wild type amino acids. For the stability objective, predictions that recapitulate known chemical phenomena such as salt bridges, hydrogen bonding, proline capping were prioritized. Process for the manual curation of Nb4OMT variants:
S188:
SER: <0.01 ARG: 0.15 MET: 0.12
ARG and/or MET may have the opportunity to interact with the phenol of norbelladine.
Rank: MET>ARG
A53:
ALA: 0.01 MET: 0.37 ARG: 0.38

MutComputeX prefers MET in the presence of ligand/cofactor, and it prefers ARG with no ligand/cofactor. ARG and MET may form either a Cation-pi interaction or a Sulfur-pi interaction with the catechol ring of norbelladine, respectively.
Rank: MET >ARG
E49:
GLU: 0.07 PRO: 0.66

MutComputeX strongly predicts PRO at the end of an alpha helix and the beginning of a loop involved in ligand binding.
RANK: PRO
E201:
GLU: 0.05 GLN: 0.38 ARG: 0.13 HIS: 0.14 LYS: 0.11

MutComputeX predicts to turn this acid into an amide or into a cation. May interact with the docked phenolic ring of Norbelladine. May form cation-pi interaction or pi-pi interactions.
Rank: GLN>HIS>LYS
W50:
TRP: 0.01 ASN: 0.20 LEU: 0.22 HIS: 0.18

MutComputeX strongly dislikes TRP in both chains. Predicts ASN in one and LEU in the other. HIS is also better predicted compared to TRP.
RANK: HIS>ASN>LEU
S52:
SER: 0.08 THR: 0.67

Net strongly predicts mutating to a THR. SER is directly contacting the amine in norbelladine.
Rank: THR
S227:
SER: 0.04 PRO: 0.77

Net strongly predicts a PRO at the end of a beta strand and beginning of a loop. Can potentially form a salt bridge with D58 of the adjacent protomer in the homodimer. In a hydrophobic pocket, so it might also be worth trying LEU.
Rank: PRO>LYS>LEU
A162:
ALA: 0.02 VAL: 0.22 PRO: 0.13 ILE: ARG: 0.08 LYS: 0.7

At the interface of two alpha helices and is semi-solvent exposed.
RANK: VAL>ARG>PRO
R237:
ARG: 0.07 VAL: 0.85
Net strongly predicts a VAL.
Rank: VAL
V11:
VAL: 0.01 LYS: 0.21
Might form a salt bridge with E47.
Rank: LYS
S159:
SER: 0.05 GLU: 0.44
Net strongly predicts GLU.
Rank: GLU
V203:
VAL: 0.02 GLU: 0.17 LYS: 0.21

MutComputeX predicts either a GLU or LYS depending on the protomer. It is worth trying both.
Rank: LYS>GLU
Y151:
TYR: 0.07 TRP: 0.89

MutComputeX strongly predicts mutating to TRP. This is in a hydrophobic pocket in the core of the protein. TRP is a more hydrophobic aromatic.
Rank: TRP>PHE
H17:
HIS: 0.09 ARG: 0.45 LYS: 0.23

At the interface of two protomers in the homodimer complex. Net strongly predicts a cation here.
Rank: ARG>LYS
E36P & G40E:

MutComputeX strongly predicts a PRO at position 36 to cap the alpha helix. However, at position 40 in the crystal structure of the 1SUI homolog there is a GLU that can form a salt bridge with K118. By removing GLU at E36 the salt bridge will be lost. By making the G40E substitution together with E36P, we can preserve the salt bridge and proline cap the alpha helix.

### High-Performance Liquid Chromatography Analysis

Assay samples were filtered using a 0.2-um PTFE syringe filter prior to running the HPLC. The measurement of Norbelladine and 4-O'Methyl-norbelladine was performed using a Vanquish HPLC system (Thermo Fisher Scientific) equipped with a BDS Hypersil TM C18 (3.0×150 mm 2, 3 um) (Thermo Fisher Scientific) with detection wavelength 277 nm. The mobile phase consisted of 0.1% formic acid in water or 0.1% formic acid in acetonitrile over the course of 28 minutes under the following conditions: 10% organic (vol/vol) for 2 minutes, 10 to 30% organic (vol/vol) for 13 minutes, 30 to 90% organic (vol/vol) for 0.1 minutes, 90% organic (vol/vol) for 4.9 minutes, 90 to 10% organic (vol/vol) for 1 minute, and 10% organic (vol/vol) for 7 minutes. The flow rate was fixed at 0.8 ml min-1. A standard curve for norbelladine was prepared using synthesized norbelladine (see Chemical synthesis and NMR analysis of norbelladine). A standard curve for 4-O'Methyl-norbelladine was prepared using commercially available 4-O'Methyl-norbelladine.

Reactions for kinetics measurements were performed in triplicate for all enzyme variants. For each variant 1.5 ml reactions containing 3.5 nM of enzyme, 500 μM SAM, 2 mM CaCl₂), and 15.625, 31.25, 62.5, 125, 250, or 500 μM norbelladine in PBS pH 7.5 were incubated at 37° C. for 4 hours. Every hour a 200 μl aliquot of each reaction was quenched by pipetting it into a 1.5 ml microcentrifuge tube with 20 μl of 2M HCl. The concentration of 4′-O-Methylnorbelladine was then determined using HPLC as described.

### Liquid Chromatography—Mass Spectrometry

Cells containing the plasmid expressing each Nb4OMT variant with the P150-RBS (RiboJ) promoter were transformed and plated onto an LB agar plate containing appropriate antibiotics. The following day, three colonies from each plate were cultured overnight in LB and subsequently diluted 50-fold into 1 ml LB containing 1 mM norbelladine. These cultures were grown for 24 h at 37° C. and centrifuged at 16,000 g for 1 min, and the resulting supernatant was filtered using a 0.2-μm filter.

Samples were analyzed using an Agilent 6530 Q-TOF LC-MS with a dual Agilent Jet Stream electrospray ionization source in positive mode. Chromatographic separations were obtained under gradient conditions by injecting 10 μl onto an Agilent RRHD Eclipse Plus C18 column (50×2.1 mm, 1.8-μm particle size) with an Agilent ZORBAX Eclipse Plus C18 narrow-bore guard column (12.5×2.1 mm, 5-μm particle size) on an Agilent 1260 Infinity II liquid chromatography system. The mobile phase consisted of eluent A (water with 0.1% formic acid) and eluent B (acetonitrile). The gradient was as follows: Hold 95% A/5% B from 0 to 2 min (0.7 ml min-1), 80% A/20% B from 2 to 15 min (0.7 ml min-1), 70% A/5% B from 15 to 18 min (0.7 ml min-1). The sample tray and column compartment were set to 7° C. and 30° C., respectively. The fragmentor was set to 100V. Q-TOF data were processed using the Agilent MassHunter Qualitative Analysis software. Both products and the residual substrate of the wildtype reactions were identified with MS/MS with a collision cell energy of 5 V.

To create the chromatograms (shown in FIG. 4B and FIG. 10), signal counts from the EIC within a window ±0.05 min relative to the retention time of the substrate and products were extracted for each scan (m/z ratios 260.1281 and 274.1438).

### Enzyme Kinetics Calculations

Kinetic data were fit in KinTek Explorer simulation and data fitting software v11 (Roy, 2018). The following minimal model was used as an input. Each line represents a step in the model and the forward reaction goes from left to right while the reverse reaction goes from right to left as written.
(1) E+S=ES
(2) ES=EP
(3) EP=E+P
(4) S=P2

Starting concentrations were entered into the software just as the reactions were performed:

3.5 nM enzyme and 15.625, 31.25, 62.5, 125, 250, and 500 M substrate. The output observable was defined as EP+P. Substrate oxidation was modeled in step (4) as irreversible with a best fit value from globally fitting data from all variants to derive k₄=0.00547 min⁻¹.To get kₒᵤₜ/Kₘ and k_{cat}: k₋₁, k₋₂, and k₋₃ were locked at 0 min-1 (irreversible reactions). k+3 was locked at 10,000 min-1 as to not limit the rate of turnover. k₊₁ and k₊₂ were used as variable parameters in the fitting. Under these conditions, k₊₂=k_{cat} and k₊₁=k_{cat}/Kₘ. For estimates of 95% confidence intervals on kinetic parameters, confidence contour analysis was used with the FitSpace function in KinTek Explorer (Bhattacharya, 2015). Confidence contour plots are calculated by systematically varying a single rate constant and holding it fixed at a particular value while refitting the data, allowing other rate constants to float. The goodness of fit was scored by the resulting χ² value. The confidence interval is defined based on a threshold in χ² calculated from the F-distribution based on the number of data points and number of variable parameters to give the 95% confidence limits. For the data given in FIGS. 20A-20C, this threshold was 0.85 to estimate the upper and lower limits for each parameter. While the model described above is the simplest model that could describe the data and gave reasonable estimates for k_{cat} and k_{cat}/Kₘ, there was evidence for substrate inhibition at the highest norbelladine concentration for the two variants (A53M and triple mutant) that this model did not account for. We then fit the data for these two variants to the model shown below, accounting for substrate inhibition.

$$E + S = ES \qquad ( 1 )$$

$$ES = EP \qquad ( 2 )$$

$$EP = E + P \qquad ( 3 )$$

$$E + S = S E \qquad ( 4 )$$

$$SE + S = S E S \qquad ( 5 )$$

$$ES + S = S E S \qquad ( 6 )$$

$$S = P 2 \qquad ( 7 )$$

As before, k₊₁ was allowed to float in the fitting to give k_{cat}/Kₘ, and k₋₁ was locked at 0 min⁻¹, k₊₂ was allowed to float in the fitting to give k_{cat}, and k₋₂ was locked at 0 min⁻¹. k₊₃ was locked at 10,000 min⁻¹, and k₋₃ was locked at 0 min⁻¹. k₊₄ and k₊₆ were locked at 100 μM⁻¹ min⁻¹, and k₋₄ and k₋₆ were allowed to float in the fitting as linked parameters. k₊₅ was linked to k₊₁ and k₋₅ was locked at 0 min⁻¹. k₋₇ was locked at 0 min⁻¹, and k₊₇ was locked at 0.00547 min⁻¹. With limited inhibition at the highest substrate concentrations tested, confidence contour analysis showed that only lower limits on k_{cat}, k_{cat}/Kₘ, and substrate inhibition could be obtained from the analysis, and these limits are reported in Table 7.

### Protein Expression and Purification

For bacterial overexpression of Nb4OMT wild type and its variants (A53M and E36P+G40E+A53M), E. coli BL21 (DE3) was used as the expression host and its competent cell was transformed with the corresponding constructed plasmids. A single colony of an E. coli BL21 (DE3) strain harboring one of the constructed plasmids was inoculated into 2 mL of Luria Bertani broth (LB) medium with 100 μg/mL ampicillin and grown overnight at 37° C./225 rpm. The overnight-grown culture (using 1 mL) was scaled up into a 500-mL autoinduction media at 37° C./225 rpm. Protein expression was automatically induced and cells were cultured for 24 hrs at 25° C./225 rpm. The induced cell culture was harvested by centrifugation at 4,000 g and 4° C. for 20 mins. Cell pellets were then resuspended in 200 mL of lysis buffer (50 mM TRIS pH 8.0, 500 mM NaCl, 20 mM Imidazole, 10% Glycerol, 10 mM β-mercaptoethanol, and 0.1% Triton-X). Cells were lysed by sonication and the resulting cell lysate was centrifuged at 15,000 g and 4° C. for 20 mins to obtain the supernatant that contains soluble proteins. The supernatant was equilibrated with HisPur™ Ni-NTA Resin (Thermo Fisher Scientific, Waltham, MA) and washed with 10× bed volumes of wash buffer (50 mM TRIS pH 8.0, 500 mM NaCl, 20 mM Imidazole, 10% Glycerol, 10 mM B-mercaptoethanol). Then protein was eluted by using a 10 mL elution buffer (50 mM TRIS pH8.0, 500 mM NaCl, 250 mM Imidazole, 10% Glycerol, 10 mM β-mercaptoethanol). The eluate was dialyzed with 3C protease added to the dialysis cassette, into the appropriate buffer (20 mM TRIS pH 7.5, 100 mM NaCl, 10 mM B-mercaptoethanol) followed by size-exclusion fast protein liquid chromatography. All Nb4OMT variants were stored in 20 mM Tris (pH 7.5), 100 mM NaCl and 10 mM B-mercaptoethanol.

### Protein X-ray Crystallography

To identify crystallization conditions of the Nb4OMT variant with triple mutations (E36P+G40E+A53M), 20 mg/ml purified enzyme samples were directly used in sparse matrix screening. Rod-shaped crystals formed after incubating screening plates at room temperature for 3 days. A crystallization condition with the best crystal morphology (0.1M Calcium Acetate, 0.1M MES pH6.5, and 20% PEG3350) was chosen and further optimized by manually setting sitting-drop vapor diffusion experiments by varying pH and precipitant concentration, resulting diffraction-quality single crystals in 0.1M Calcium Acetate, 0.1M MES pH 7.0, and 26% PEG3350.

Individual Nb4OMT variant (E36P+G40E+A53M) crystals were flash-frozen directly in liquid nitrogen after brief incubation with a reservoir solution supplemented with 30% (v/v) glycerol. X-ray diffraction data were collected at BL 8.2.2 in ALS (Berkeley, CA). X-ray diffraction data were processed to 2.4 Å using HKL2000. In Phenix software, phases were obtained by molecular replacement using an AlphaFold2 model of Nb4OMT as the initial search model. The molecular replacement solution was iteratively built and refined using Coot and Phenix refine package. The quality of the final refined structures was evaluated by MolProbity. The final statistics for data collection and structure determination are shown in FIG. 16.

### Differential Scanning Fluorimetry

Purified Nb4OMT variants in the concentration of 5 μM were prepared in 96-well low-profile PCR plates (ABgene, Thermo Scientific). 10×SYPRO® Orange (Molecular Probes) was added into each well and mixed prior to measurement in an RT-PCR machine (LightCycler 480, Roche). The protein melting experiments were carried out with a continuous temperature acquisition mode using 10 acquisitions per 1° C. in each cycle from 20° C. to 95° C. The melting curves of the Nb4OMT variants were monophasic and Tₘ values were derived using Boltzmann equation.

### Statistical Analysis and Reproducibility

All data in the text are displayed as mean±s.e.m. unless specifically indicated. Bar graphs, fluorescence and growth curves, dose-response functions were all plotted in Python 3.6.9 using Matplotlib. Dose-response curves and EC₅₀ values were estimated by fitting to the Hill equation y=d+(a−d)xb(cb+xb)−1 (where y=output signal, b=Hill coefficient, x=ligand concentration, d=background signal, a=maximum signal and c=EC₅₀), with the scipy.optimize.curve_fit library in Python.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the claims.

SEQUENCES

SEQ. ID. NO: 1
>pSens4NB2 nucleic acid
TGGTTCGTTGTGATGGCGGTAGGAATGTAATCGTTAATCCGCAAATAACGTA
AAAACCCGCTTCGGCGGGTTTTTTTATGGGGGGAGTTTAGGGAAAGAGCATTTGTCA
TCCCGTTGAATATGGCTCGCATCTTATCGAGCATACTATCACGTCGGCGACCACTAG
TCAGTTAACGCAAGGGCATGGGCTGTCGACCTTTGAAAAGTACCTTGACGGCGTATC
TTTGCTTTCTATAATGAGTGCTTACTCACTCATACAATAGTCAGTCATAAGTCTGGGC
TAAGCCCACTGATGAGTCGCTGAAATGCGACGAAACTTATGACCTCTACAAATAATT
TTGTTTAACGTAAACCTCCGGGTTAATAAGGAGTAATTATGGCATCCAAGGGCGAG
GAGCTCTTTACTGGCGTAGTACCAATTCTCGTAGAGCTCGATGGCGATGTAAATGGC
CATAAGTTTTCCGTACGCGGCGAGGGCGAGGGCGATGCAACTAACGGCAAGCTCAC
TCTCAAGTTTATTTGTACTACTGGCAAGCTCCCAGTACCATGGCCAACTCTCGTAAC
TACTCTGACCTATGGCGTACAATGTTTTTCCCGCTATCCAGATCACATGAAGCAACA
TGATTTTTTTAAGTCCGCAATGCCAGAGGGCTATGTACAAGAGCGCACTATTAGCTT
TAAGGATGATGGCACCTATAAGACTCGCGCAGAGGTAAAGTTTGAGGGCGATACTC
TCGTAAATCGCATTGAGCTCAAGGGCATTGATTTTAAGGAGGATGGCAATATTCTCG
GCCATAAGCTGGAGTATAATTTCAATTCCCATAATGTATATATTACCGCAGATAAGC
AAAAGAATGGCATTAAGGCGAATTTTAAGATTCGCCATAATGTGGAGGATGGCTCC
GTACAACTCGCAGATCATTATCAACAAAATACTCCAATTGGCGATGGCCCAGTACTC
CTCCCAGATAATCATTATCTCTCCACTCAATCCGTGCTCTCCAAAGATCCAAATGAG
AAGCGCGATCACATGGTACTCCTGGAGTTTGTAACTGCAGCAGGCATTACTCATGGC
ATGGATGAGCTCTATAAGCTCGAGCACCACCACCACCACCACTGATAATCCAAACC
TGTTATATGTTAGCTGAGACTAGTTGGAAGTGTGGCTGTCCTCAAGCGTTTTAGTTC
GTCGGTCAGTTTCACCTGATTTACGTAAAAACCCGCTTCGGCGGGTTTTTGCTTTTGG
AGGGGCAGAAAGATGAATGACTGTCGGCCATTCGATGGTGTCGGGTAGCATAACCC
CTTGTGATACCTTTGCCATGTTTCAGAAACAACTCTGGCGCATCGGGCTTGGACCAA
AACGAAAAAAGGCCGCTTTCGCGGCCTCTTTTCTGGAATTTGGTACCGAGCTCACAG
CGGCGATAGTCAGATAGCTAGACCGTATGTTACCGAGCCTGCACTCCTCGAATTCTT
AGGATTATTACTGCTCTTCGCGCGTAAGTGCGCGCCACATAGCCTCGAAGCCCAACG
CAATGTACTCACCAGCGCGAGCCGGGTCGCGCGCAGCGAAATCCATAGTCGTCTCA
GCAAGCGCCAAGAACAACCCGTCGCCGAAGGCGCGGTACTCGTCGGACATAAACAC
CATAAGAACGCCACGGTGGTCGAGGTCGCGTAACTCCGGGAACATATCATCCGCGC
GTTGTTCGGTTTCCTTCGTCAACTTTTCAGAAACCGCCAACTGACGAATGGCACGAT
GGCGAGCTGGGTGGTTCAATCCCCAGCTAATATAACTGTTCCAGATAAAACGGGTC
ATCATCTTAGCGTCAGTAATAGAACGATCCAATTCCATGATCATTGATTGGCACATG
TCCTGGGTCAAATGTAAGTAAAGGGTGTTGATCAACTCATCTTTCGTTGCGAAATAG
CGGAACAACGTCCCTTCCGCAACTCCCGCATTGCGTGCAATTACAGCGGTACTAGCG
GCAATGCCTGATTGCGCGATGGCTTGAGTTGCCGCTTCAAGCAATGCCTGCTTTTTG
TCCTCAGACTTTGGGCGAGCAACCATATACTAACCTCCTTCTGATACGTGGTTCCGT
TAAACAAAATTATTTGTAGAGGCCCCATTTCGTCCTTTTGGACTCATCAGGGGTGGT
ACACACCACCCTATGGGGCTCGTAATTGCTAGCATAATCCCTAGGACTGAGCTAGCT
ATCAGGGTACTTTTCAAAGGTCGACAGCCCATGCCCTTGCGTTCGGCAGGTGTACAA
TGATACGAGGTAATGAAGATGAAGTCCATACAATCGATAGATTGGGACCAAAACGA
AAAAAGGGGAGCGGTTTCCCGCTCCCCTCTTTTCTGGAATTTGGTACCGAGTCGCAC
CTGATTGCCCGACATTATCGCACGGTGTCTCATCTCTGATAACGCATATTGTCGTTA
GAACTCGGCGCGGCCGCTCACACTGCTTCCGGTAGTCAATAAACCGGTAAACCAGC
AATAGACATAAGCGGCTATTTAACGACCCTGCCCTGAACCGACGACCGGGTCGAAT
TTGCTTTCGAATTTCTGCCATTCATCCGCTTATTATCACTTATTCAGGCGTAGCAACC
AGGCGTTTAAGGGCACCAATAACTGCCTTAAAAAAATTAGAAAAACTCATCGAGCA
TCAAATGAAACTGCAATTTATTCATATCAGGATTATCAATACCATATTTTTGAAAAA
GCCGTTTCTGTAATGAAGGAGAAAACTCACCGAGGCAGTTCCATAGGATGGCAAGA
TCCTGGTATCGGTCTGCGATTCCGACTCGTCCAACATCAATACAACCTATTAATTTCC
CCTCGTCAAAAATAAGGTTATCAAGTGAGAAATCACCATGAGTGACGACTGAATCC
GGTGAGAATGGCAAAAGTTTATGCATTTCTTTCCAGACTTGTTCAACAGGCCAGCCA
TTACGCTCGTCATCAAAATCACTCGCATCAACCAAACCGTTATTCATTCGTGATTGC
GCCTGAGCGAGACGAAATACGCGGTCGCTGTTAAAAGGACAATTACAAACAGGAAT
CGAATGCAACCGGCGCAGGAACACTGCCAGCGCATCAACAATATTTTCACCTGAAT
CAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTT
TGCCATGTTTCAGAAACAACTCTGGCGCATCGGGCTTCCCATACAATCGATAGATTG
TCGCACCTGATTGCCCGACATTATCGCGAGCCCATTTATACCCATATAAATCAGCAT
CCATGTTGGAATTTAATCGCGGCCTAGAGCAAGACGTTTCCCGTTGAATATGGCTCA
TTTTAGCTTCCTTAGCTCCTGAAAATCTCGATAACTCAAAAAATACGCCCGGTAGTG
ATCTTATTTCATTATGGTGAAAGTTGGAACCTCTTACGTGCCGATCACGTCTCATTTT
CGCCAAAGTTGGCCAGGGCTTCCCGGTATCAACAGGGACACCAGGATTTATTTATN
NTGCGAAGTGATCTTCCGTCACAGGTATTTATTCGGCGCAAAGTGCGTCGGGTGATG
CTGCCAACTTACTGATTTAGTGTATGATGGTGTTTTTGAGGTGCTCCAGTGGCTTCTG
TTTCTATCAGCTGTCCCTCCTGTTCAGCTACTGACGGGGTGGTGCGTAACGGCAAAA
GCACCGCCGGACATCAGCGCTAGCGGAGTGTATACTGGCTTACTATGTTGGCACTGA
TGAGGGTGTCAGTGAAGTGCTTCATGTGGCAGGAGAAAAAAGGCTGCACCGGTGCG
TCAGCAGAATATGTGATACAGGATATATTCCGCTTCCTCGCTCACTGACTCGCTACG
CTCGGTCGTTCGACTGCGGCGAGCGGAAATGGCTTACGAACGGGGCGGAGATTTCC
TGGAAGATGCCAGGAAGATACTTAACAGGGAAGTGAGAGGGCCGCGGCAAAGCCG
TTTTTCCATAGGCTCCGCCCCCCTGACAAGCATCACGAAATCTGACGCTCAAATCAG
TGGTGGCGAAACCCGACAGGACTATAAAGATACCAGGCGTTTCCCCCTGGCGGCTC
CCTCGTGCGCTCTCCTGTTCCTGCCTTTCGGTTTACCGGTGTCATTCCGCTGTTATGG
CCGCGTTTGTCTCATTCCACGCCTGACACTCAGTTCCGGGTAGGCAGTTCGCTCCAA
GCTGGACTGTATGCACGAACCCCCCGTTCAGTCCGACCGCTGCGCCTTATCCGGTAA
CTATCGTCTTGAGTCCAACCCGGAAAGACATGCAAAAGCACCACTGGCAGCAGCCA
CTGGTAATTGATTTAGAGGAGTTAGTCTTGAAGTCATGCGCCGGTTAAGGCTAAACT
GAAAGGACAAGTTTTGGTGACTGCGCTCCTCCAAGCCAGTTACCTCGGTTCAAAGA
GTTGGTAGCTCAGAGAACCTTCGAAAAACCGCCCTGCAAGGCGGTTTTTTCGTTTTC
AGAGCAAGAGATTACGCGCAGACCAAAACGATCTCAAGAAGATCATCTTATTAATC
AGATAAAATATTTCTAGATTTCAGTGCAATTTATCTCTTCAAATGTAGCACCTGAAG
TCAGCCCCATACGATATAAGTTGTAATTCTCATGTTAGTCATGCCCCGCGCCCACCG
GAAGGAGCTGACTGGGTTGAAGGCTCTCAAGGGCATCGGTCGAGATCCCGGTGCCT
AATGAGTGAGCTAACTTACATTAATTGCGTTGCGCTCACTGCCCGCTTTCCAGTCGG
GAAACCTGTCGTGCCAGCTGCATTAATGAATCGGCCAACGCGCGGGGAGAGGCGGT
TTGCGTATTGGGCGCCAGGGTGGTTTTTCTTTTCACCAGTGAGACGGGCAACAGCTG
ATTGCCCTTCACCGCCTGGCCCTGAGAGAGTTGCAGCAAGCGGTCCACGCTGGTTTG
CCCCAGCAGGCGAAAATCCTGTTTGATGGTGGTTAACGGCGGGATATAACATGAGC
TATCTTCGGTATCGTCGTATCCCACTACCGAGATGTCCGCACCAACGCGCAGCCCGG
ACTCGGTAATGGCGCGCATTGCGCCCAGCGCCATCTGATCGTTGGCAACCAGCATCG
CAGTGGGAACGATGCCCTCATTCAGCATTTGCATGGTTTGTTGAAAACCGGACATGG
CACTCCAGTCGCCTTCCCGTTCCGCTATCGGCTGAATTTGATTGCGAGTGAGATATTT
ATGCCAGCCAGCCAGACGCAGACGCGCCGAGACAGAACTTAATGGGCCCGCTAACA
GCGCGATTTGCTGGTGACCCAATGCGACCAGATGCTCCACGCCCAGTCGCGTACCAT
CTTCATGGGAGAAAATAATACTGTTGATGGGTGTCTGGTCAGAGACATCAAGAAAT
AACGCCGGAACATTAGTGCAGGCAGCTTCCACAGCAATGGCATCCTGGTCATCCAG
CGGATAGTTAATGATCAGCCCACTGACGCGTTGCGCGAGAAGATTGTGCACCGCCG
CTTTACAGGCTTCGACGCCGCTTCGTTCTACCATCGACACCACCACGCTGGCACCCA
GTTGATCGGCGCGAGATTTAATCGCCGCGACAATTTGCGACGGCGCGTGCAGGGCC
AGACTGGAGGTGGCAACGCCAATCAGCAACGACTGTTTGCCCGCCAGTTGTTGTGC
CACGCGGTTGGGAATGTAATTCAGCTCCGCCATCGCCGCTTCCACTTTTTCCCGCGTT
TTCGCAGAAACGTGGCTGGCCTGGTTCACCACGCGGGAAACGGTCTGATAAGAGAC
ACCGGCATACTCTGCGACATCGTATAACGTTACTGGTTTCACATTCACCACCCTGAA
TTGACTCTCTTCCGGGCGCTATCATGCCATACCGCGAAAGGTTTTGCGCCATTCGAT
GGTGTCCGGGATCTCGACGCTCTCCCTTATGCGACGCGGCCGCGGCATCAGAGCAG
ATTGTACTGTGTCCTCAA
SEQ ID NO: 2
>Nb4OMT-A53M nucleic acid
ATCCCCCTTACACGGAGGCATCAGTGACCAAACAGGAAAAAACCGCCCTTAA
CATGGCCCGCTTTATCAGAAGCCAGACATTAACGCTTCTGGAGAAACTCAACGAGC
TGGACGCGGATGAACAGGCAGACATCTGTGAATCGCTTCACGACCACGCTGATGAG
CTTTACCGCAGCTGCCTCGCGCGTTTCGGTGATGACGGTGAAAACCTCTGACACATG
CAGCTCCCGCAGACGGTCACAGCTTGTCTGTAAGCGGATGCCGGGAGCAGACAAGC
CCGTCAGGGCGCGTCAGCGGGTGTTGGCGGGTGTCGGGGCGCAGCCATGACCCAGT
CACGTAGCGATAGCGGAGTGTATACTGGCTTAACTATGCGGCATCAGAGCAGATTG
TACTGAGAGTGCACCGGTGTGAAATACCGCACAGATGCGTAAGGAGAAAATACCGC
ATCAGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGG
GATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTA
CACTAGAAGGACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTT
TGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGCCCTCTCC
AAGACCGAGCCATCAACAAAGCGTCTCGCTGAGGTTTCATGGAGCCTCTGGTTCATC
TCCGGCAATTAAAAAAGCGGCTAACCACGCCGCTTTTTTTACGTCTGCAGGAACGGG
CTGTCGACCTTTGAAAAGTTCGTTTACCGCTAGCTCAGTCCTAGGTACAATTACAGC
CATCGTACGAGCCCTGGCTGAGCACAGCTGTCACCGGATGTGCTTTCCGGTCTGATG
AGTCCGTGAGGACGAAACAGCCTCTACAAATAATTTTGTTTAAACTAGTGAACCAC
GAGGCCTACATATGGGTGCTTCAATTGATGACTACTCCTTGGTACATAAAAACATCT
TGCATTCCGAAGATCTGCTGAAATACATTCTTGAAACCAGTGCATATCCTCGCGAAC
ACGAACAATTAAAGGGTCTGCGTGAGGTTACTGAAAAGCACGAATGGTCATCCatgT
TAGTACCGGCAGACGAAGGTTTATTCCTGTCAATGTTACTGAAGTTGATGAATGCAA
AACGTACTATCGAAATCGGCGTGTACACGGGATACAGCCTGTTAACAACGGCATTG
GCTTTACCGGAGGATGGTAAAATTACGGCGATCGATGTAAATAAGAGTTATTACGA
AATCGGATTGCCCTTTATTCAGAAGGCGGGCGTGGAACACAAGATCAACTTTATTGA
GTCGGAAGCGCTTCCCGTGCTGGATCAAATGTTAGAGGAGATGAAGGAGGAGGATT
TATACGACTACGCTTTTGTGGATGCTGATAAAAGCAATTATGCCAATTACCATGAGC
GTCTTGTAAAACTTGTACGTATCGGTGGTGCCATCCTGTACGACAATACACTGTGGT
ATGGTTCTGTTGCGTACCCGGAATACCCCGGTCTGCATCCAGAGGAAGAAGTCGCG
CGTCTGAGCTTTCGTAACTTGAATACCTTTTTAGCAGCAGATCCTCGCGTAGAAATT
AGTCAAGTCTCAATTGGTGATGGCGTGACCATTTGTCGCCGCTTGTATTAATAATCC
TATCGCCACTTTCAGCCAAAAAACTTAAGACCGCCGGTCTTGTCCACTACCTTGCAG
TAATGCGGTGGACAGGATCGGCGGTTTTCTTTTCTCTTCTCAACACCCTTCGCGTCAA
CACTTTTCCGCCAAGGAGACGGTTGGTCAGGTTTTCGGGAGGTGTGGCTGGAAGTTC
CTATACTTTCTAGAGAATAGGAACTTCTTTCTAAATACATTCAAATATGTATCCGCTC
ATGAGACAATAACCCTGATAAATGCTTCAATAATATTGAAAAAGGAAGAGTATGAG
TATTCAACATTTCCGTGTCGCCCTTATTCCCTTTTTTGCGGCATTTTGCCTTCCTGTTT
TTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGC
CCCGAAGAACGTTTTCCAATGATGAGCACTTTTAAAGTTCTGCTATGTGGCGCGGTA
TTATCCCGTGTTGACGCCGGGCAAGAGCAACTCGGTCGCCGCATACACTATTCTCAG
AATGACTTGGTTGAGTACTCACCAGTCACAGAAAAGCATCTTACGGATGGCATGAC
AGTAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATACC
AAACGACGAGCGTGACACCACGATGCCTGCAGCAATGGCAACAACGTTGCGCAAAC
TATTAACTGGCGAACTACTTACTCTAGCTTCCCGGCAACAATTAATAGACTGGATGG
AGGCGGATAAAGTTGCAGGACCACTTCTGCGCTCGGCCCTTCCGGCTGGCTGGTTTA
TTGCTGATAAATCTGGAGCCGGTGAGCGTGGATCGCGCGGTATCATTGCAGCACTG
GGGCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAAGC
ATTGGTAAGTTGTGATGGCGGTAGGAATGTAATCGTTAATCCGCAAATAACGTAAA
AACCCGCTTCGGCGGGTTTTTTTATGGGGGGAGTTTAGGGAAAGAGCATTTGTCATC
CCGTTGAATATGGCTCCCTTAACGTGAGGAAGTTCCTATACTTTCTAGAGAATAGGA
ACTTCTACAGATGGACTTGGGTTGGCGGTTTCAGGAGTCTGCAAAACGTCTGCGACC
TGAGCAACAACATGAATGGTCATCGGTTTCCGTGTTTCGTAAAGTCTGGAAACGCGG
AAGTCAGCGCCCTGCACCATTATGTTCCGGATCTGCATCGCAGGATGCTGCTGGCTA
CCCTGTGGAACACCTACATCTGTATTAACGAAGCGCTGGCATTGACCCTGAGTGATT
TTTCTCTGGTCCCGCCGCATCCATACCGCCAGTTGTTTACCCTCACAACGTTCCAGTA
ACCGGGCATGTTCATCATCAGTAACCCGTATCGTGAGCATCCTCTCTCGTTTCATCG
GTATCATTACCCCCATGAACAGAA
SEQ ID NO: 3: Natural enzyme (norbelladine 4′-O-Methyltransferase)
MGASIDDYSLVHKNILHSEDLLKYILETSAYPREHEQLKGLREVTEKHEWSSALV
PADEGLFLSMLLKLMNAKRTIEIGVYTGYSLLTTALALPEDGKITAIDVNKSYYEIGLPFI
QKAGVEHKINFIESEALPVLDQMLEEMKEEDLYDYAFVDADKSNYANYHERLVKLVRI
GGAILYDNTLWYGSVAYPEYPGLHPEEEVARLSFRNLNTFLAADPRVEISQVSIGDGVTI
CRRLY
Improved enzyme variants (SEQ ID NOS: 4-8, 17 and 18)
SEQ ID NO: 4: A53M
MGASIDDYSLVHKNILHSEDLLKYILETSAYPREHEQLKGLREVTEKHEWSSML
VPADEGLFLSMLLKLMNAKRTIEIGVYTGYSLLTTALALPEDGKITAIDVNKSYYEIGLP
FIQKAGVEHKINFIESEALPVLDQMLEEMKEEDLYDYAFVDADKSNYANYHERLVKLV
RIGGAILYDNTLWYGSVAYPEYPGLHPEEEVARLSFRNLNTFLAADPRVEISQVSIGDGV
TICRRLY
SEQ ID NO: 5: S159E
MGASIDDYSLVHKNILHSEDLLKYILETSAYPREHEQLKGLREVTEKHEWSSALV
PADEGLFLSMLLKLMNAKRTIEIGVYTGYSLLTTALALPEDGKITAIDVNKSYYEIGLPFI
QKAGVEHKINFIESEALPVLDQMLEEMKEEDLYDYAFVDADKENYANYHERLVKLVRI
GGAILYDNTLWYGSVAYPEYPGLHPEEEVARLSFRNLNTFLAADPRVEISQVSIGDGVTI
CRRLY
SEQ ID NO: 6: V203E
MGASIDDYSLVHKNILHSEDLLKYILETSAYPREHEQLKGLREVTEKHEWSSALV
PADEGLFLSMLLKLMNAKRTIEIGVYTGYSLLTTALALPEDGKITAIDVNKSYYEIGLPFI
QKAGVEHKINFIESEALPVLDQMLEEMKEEDLYDYAFVDADKSNYANYHERLVKLVRI
GGAILYDNTLWYGSVAYPEYPGLHPEEEEARLSFRNLNTFLAADPRVEISQVSIGDGVTI
CRRLY
SEQ ID NO: 7: H17K
MGASIDDYSLVHKNILKSEDLLKYILETSAYPREHEQLKGLREVTEKHEWSSALV
PADEGLFLSMLLKLMNAKRTIEIGVYTGYSLLTTALALPEDGKITAIDVNKSYYEIGLPFI
QKAGVEHKINFIESEALPVLDQMLEEMKEEDLYDYAFVDADKSNYANYHERLVKLVRI
GGAILYDNTLWYGSVAYPEYPGLHPEEEVARLSFRNLNTFLAADPRVEISQVSIGDGVTI
CRRLY
SEQ ID NO: 8: E36P-G40E
MGASIDDYSLVHKNILHSEDLLKYILETSAYPREHPQLKELREVTEKHEWSSALV
PADEGLFLSMLLKLMNAKRTIEIGVYTGYSLLTTALALPEDGKITAIDVNKSYYEIGLPFI
QKAGVEHKINFIESEALPVLDQMLEEMKEEDLYDYAFVDADKSNYANYHERLVKLVRI
GGAILYDNTLWYGSVAYPEYPGLHPEEEVARLSFRNLNTFLAADPRVEISQVSIGDGVTI
CRRLY
SEQ ID NO: 17: H17R
MGASIDDYSLVHKNILRSEDLLKYILETSAYPREHEQLKGLREVTEKHEWSSALV
PADEGLFLSMLLKLMNAKRTIEIGVYTGYSLLTTALALPEDGKITAIDVNKSYYEIGLPFI
QKAGVEHKINFIESEALPVLDQMLEEMKEEDLYDYAFVDADKSNYANYHERLVKLVRI
GGAILYDNTLWYGSVAYPEYPGLHPEEEVARLSFRNLNTFLAADPRVEISQVSIGDGVTI
CRRLY
SEQ ID NO: 18: E36P-G40E-A53M
MGASIDDYSLVHKNILHSEDLLKYILETSAYPREHPQLKELREVTEKHEWSSML
VPADEGLFLSMLLKLMNAKRTIEIGVYTGYSLLTTALALPEDGKITAIDVNKSYYEIGLP
FIQKAGVEHKINFIESEALPVLDQMLEEMKEEDLYDYAFVDADKSNYANYHERLVKLV
RIGGAILYDNTLWYGSVAYPEYPGLHPEEEVARLSFRNLNTFLAADPRVEISQVSIGDGV
TICRRLY
Natural biosensor: SEQ ID NO: 9
MVARPKSEDKKQALLEAATQAIAQSGIAASTAVIARNAGVAEGTLFRYFATKDE
LINTLYLHLKQDLCQSMIMELDRSITDAKMMTRFIWNSYISWGLNHPARHRAIRQLAVS
EKLTKETEQRADDMFPELRDLCHRSVLMVFMSDEYRAFGDGLFLALAETTMDFAARDP
ARAGEYIALGFEAMWRALTREEQ
Improved biosensor variants (SEQ ID NOS: 10-16)
SEQ ID NO: 10: L133T, C134E, S127T
MVARPKSEDKKQALLEAATQAIAQSGIAASTAVIARNAGVAEGTLFRYFATKDE
LINTLYLHLKQDLCQSMIMELDRSITDAKMMTRFIWNSYISWGLNHPARHRAIRQLAVS
EKLTKETEQRADDMFPELRDTEHRTVLMVFMSDEYRAFGDGLFLALAETTMDFAARD
PARAGEYIALGFEAMWRALTREEQ
SEQ ID NO: 11: K63T, L66M
MVARPKSEDKKQALLEAATQAIAQSGIAASTAVIARNAGVAEGTLFRYFATKDE
LINTLYLHLTQDMCQSMIMELDRSITDAKMMTRFIWNSYISWGLNHPARHRAIRQLAVS
EKLTKETEQRADDMFPELRDLCHRSVLMVFMSDEYRAFGDGLFLALAETTMDFAARDP
ARAGEYIALGFEAMWRALTREEQ
SEQ ID NO: 12: K63R, M70T
MVARPKSEDKKQALLEAATQAIAQSGIAASTAVIARNAGVAEGTLFRYFATKDE
LINTLYLHLRQDLCQSTIMELDRSITDAKMMTRFIWNSYISWGLNHPARHRAIRQLAVS
EKLTKETEQRADDMFPELRDLCHRSVLMVFMSDEYRAFGDGLFLALAETTMDFAARDP
ARAGEYIALGFEAMWRALTREEQ
SEQ ID NO: 13: K63T, L66M, C134D, S137G
MVARPKSEDKKQALLEAATQAIAQSGIAASTAVIARNAGVAEGTLFRYFATKDE
LINTLYLHLTQDMCQSMIMELDRSITDAKMMTRFIWNSYISWGLNHPARHRAIRQLAVS
EKLTKETEQRADDMFPELRDLDHRGVLMVFMSDEYRAFGDGLFLALAETTMDFAARD
PARAGEYIALGFEAMWRALTREEQ
SEQ ID NO: 14: K63T, L66M, C134D, S137N
MVARPKSEDKKQALLEAATQAIAQSGIAASTAVIARNAGVAEGTLFRYFATKDE
LINTLYLHLTQDMCQSMIMELDRSITDAKMMTRFIWNSYISWGLNHPARHRAIRQLAVS
EKLTKETEQRADDMFPELRDLDHRNVLMVFMSDEYRAFGDGLFLALAETTMDFAARD
PARAGEYIALGFEAMWRALTREEQ
SEQ ID NO: 15 K63T, L66M, C134E, S137D
MVARPKSEDKKQALLEAATQAIAQSGIAASTAVIARNAGVAEGTLFRYFATKDE
LINTLYLHLTQDMCQSMIMELDRSITDAKMMTRFIWNSYISWGLNHPARHRAIRQLAVS
EKLTKETEQRADDMFPELRDLCHRDVLMVFMSDEYRAFGDGLFLALAETTMDFAARD
PARAGEYIALGFEAMWRALTREEQ
SEQ ID NO: 16: K63T, L66M, C134N, S137G
MVARPKSEDKKQALLEAATQAIAQSGIAASTAVIARNAGVAEGTLFRYFATKDE
LINTLYLHLTQDMCQSMIMELDRSITDAKMMTRFIWNSYISWGLNHPARHRAIRQLAVS
EKLTKETEQRADDMFPELRDLNHRGVLMVFMSDEYRAFGDGLFLALAETTMDFAARD
PARAGEYIALGFEAMWRALTREEQ

TABLE 1
GNINA1.0 docking metrics for S-adenosyl-
Homocysteine(SAH) and Norbelladine.
TABLES
GNINA1.0 docking results

| | minimized | Minimised | | CNN |
|---|---|---|---|---|
| | Affinity | RMSD | CNN Score | Affinity |
| ligand | (Kcal/mol) | (Angstrom) | (probability) | (pK) |
| S-adenosyl- | −7.918 | 0.557 | 0.835 | 5.851 |
| Homocysteine | | | | |
| Norbelladine | −7.261 | 0.162 | 0.824 | 5.303 |

TABLE 2
Dataset composition for training/testing 3DResNet models. Dataset
consists of 22,759 protein sequences clustered at 50% sequence
similarity from the PDB. The sequences were sampled to generate
a 2,569,256 microenvironment dataset, which was then split 90:10
to generate the training and test set, respectively. A) percentage
of the dataset sampled at functional interfaces (within 5 angstroms
of a non-protein atom or randomly from the core or surface of
proteins. B) Amino acid composition of the dataset.
a)

| Interface Type | Percentage (%) |
|---|---|
| DNA | 0.04 |
| RNA | 0.72 |
| Ligand | 10.9 |
| Halogen | 1.17 |
| Protein | 23.98 |
| random core/surface | 63.19 |

b)

| Amino Acid | Percentage (%) |
|---|---|
| ALA | 7.84 |
| ARG | 5.51 |
| ASN | 4.39 |
| ASP | 6.01 |
| CYS | 1.4 |
| GLN | 3.72 |
| GLU | 6.68 |
| GLY | 7.18 |
| HIS | 2.71 |
| ILE | 5.6 |
| LEU | 9.08 |
| LYS | 5.6 |
| MET | 2.25 |
| PHE | 4.17 |
| PRO | 4.48 |
| SER | 5.89 |
| THR | 5.43 |
| TRP | 1.53 |
| TYR | 3.79 |
| VAL | 6.75 |

TABLE 3
Wildtype accuracy and Pearson and Spearman correlation metrics with ΔTm and ΔΔG
experimental values for point mutations in FireProtDB (Hekkelman, 2023). Correlations were
calculated with the log odds predicted by the deep learning model for the mutated and wildtype
$amino acids : log log ( \frac{mutAA\_probability}{wtAA\_probability} ) .$

| | FP Wild | | | | | ΔTm | ΔΔG |
|---|---|---|---|---|---|---|---|
| | Type | Pearson | Spearman | Pearson | Spearman | dataset | dataset |
| Model | Accuracy | ΔTm | ΔTm | ΔΔG | ΔΔG | size | size |
| 3DCNN | 0.628 | 0.325 | 0.369 | −0.369 | −0.412 | 2719 | 4889 |
| ensemble | | | | | | | |
| 3DResNet | 0.597 | 0.367 | 0.425 | −0.408 | −0.457 | 2719 | 4889 |
| ensemble | | | | | | | |
| 3DResNet | 0.626 | 0.332 | 0.383 | −0.381 | −0.429 | 2719 | 4889 |
| 1 | | | | | | | |
| 3DResNet | 0.656 | 0.405 | 0.426 | −0.421 | −0.452 | 2719 | 4889 |
| 2 | | | | | | | |
| 3DResNet | 0.591 | 0.378 | 0.435 | −0.423 | −0.474 | 2719 | 4889 |
| 3 | | | | | | | |

TABLE 4
Kinetic and thermal parameters of the wild-type and mutant Nb4OMTs.
Lower and upper bounds for the 95% confidence interval from confidence
contour analysis for k_{cat}/Kₘ and k_{cat} given in parentheses. Kₘ was
calculated by dividing k_{cat} by k_{cat}/Kₘ. Due to the weak substrate
inhibition term for the A53M and triple mutation variants, general
upper and lower limits on steady state kinetic parameters are reported
(see Methods).

| | k_{cal}/Kₘ | k_{cat} | | |
|---|---|---|---|---|
| Enzyme | (μM⁻¹ min⁻¹) | (min⁻¹) | Kₘ (μM) | Tₘ(° C.) |
| Wild-type | 1.18 | 73 | 62 | 52.8 |
| Nb4OMT | (0.85-1.69) | (63-89) | (37-104) | (52.8-52.8) |
| A53M mutant | >2.1 | >190 | <90 | 54.5 |
| | | | | (54.3-54.6) |
| E36P/G40E/A53M | >1.4 | >120 | <83 | 58.4 |
| mutant | | | | (58.4-58.4) |

TABLE 5
4′-O-methylnorbelladine measurements made with HPLC and
the 4NB2.1 biosensor. HPLC values represent the measured
AUC fit to a standard curve. Biosensor measurements represent
Relative Fluorescence Units divided by OD600 (RFU/OD).
Error represents the standard deviation +/− the mean.

| 4NB ligand | | | 4NB2.1 | 4NB2.1 |
|---|---|---|---|---|
| supplemented | HPLC | HPLC | biosensor | biosensor |
| into media(uM) | measurement | error | measurement | error |
| 0 | 4.25 | 7.22 | 183.85 | 10.94 |
| 1 | 2.96 | 1.28 | 200.81 | 4.13 |
| 2.5 | 8.53 | 10.15 | 240.62 | 11.25 |
| 5 | 17.02 | 10.57 | 395.69 | 19.35 |
| 10 | 22.47 | 9.78 | 944.28 | 46.35 |
| 25 | 33.94 | 9.7 | 5648.44 | 409.69 |
| 50 | 50.89 | 0.82 | 21194.72 | 716.82 |
| 100 | 122.04 | 14.18 | 43800.94 | 1702.33 |
| 250 | 260.25 | 1.5 | 64506.12 | 3283.8 |
| 500 | 523.06 | 3.62 | 67029.6 | 2473.36 |
| 1000 | 1058.36 | 7.48 | 62040.21 | 1175.72 |

TABLE 6
Genotypes and aliases of combinatorial Nb4OMT mutants.

| Mutant Alias | Genotype |
|---|---|
| 17-203 | H17K, V203E |
| 17-36 | H17K, E36P, G40E |
| 17-159 | H17K, S159E |
| 36-203 | E36P, G40E, V203E |
| 17-53 | H17K, A53M |
| 17-53-203 | H17K, A53M, V203E |
| 17-53-159 | H17K, A53M, S159E |
| 53-159 | A53M, S159E |
| 53-203 | A53M, V203E |
| 36-53 | E36P, G40E, A53M |
| 36-53-203 | E36P, G40E, A53M, V203E |
| 36-53-159 | E36P, G40E, A53M, S159E |

TABLE 7
X-ray crystallography data collection and refinement statistics.

| Data collection | | |
|---|---|---|
| Space group | P2₁ | |
| Cell dimensions | | |
| a, b, c (A) | 95.97, 79.58, 98.38 | |
| α, β, γ (°) | 90.00, 105.62, 90.00 | |
| Resolution (A) | 50.00 − 2.40 | (2.44 − 2.40)* |
| Rsym/ Rpim | 0.163(0.441)/0.095(0.278) | |
| CC ½ ^{γ} | 0.966 | (0.778) |
| I/σ | 5.6 | (1.6) |
| Completeness (%) | 99.3 | (93.9) |
| Redundancy | 3.8 | (3.2) |
| Refinement | | |
| Resolution (A) | 47.692 − 2.398 | (2.483 − 2.398) |
| No. reflections | 55735 | (5310) |
| R_{work} | 0.1891 | (0.2357) |
| R_{free}^{±} | 0.2489 | (0.3285) |

| | |
|---|---|
| No. atoms | 11657 |
| Protein | 11349 |
| Ligand/ion | 9 |
| Water | 299 |
| B-factors (A²) | |
| Protein | 23.1 |
| Ligand/ion | 22.5 |
| Water | 24.6 |
| R.m.s. deviations | |
| Bond lengths (A) | 0.0023 |
| Bond angles (°) | 0.58 |
| Ramachandran plot | |
| Favored | 97.31% |
| Allowed | 2.69% |
| Outliers | 0.00% |
| Molprobity score | 1.42/99th percentile |

*Values for the corresponding parameters in the outermost shell in parenthesis.
^{γ} CC_{1/2} is the Pearson correlation coefficient for a random half of the data, the two numbers represent the lowest and highest resolution shell respectively.
^{±}R_{free} is the R_{work} calculated for about 10% of the reflections randomly selected and omitted from refinement.

### REFERENCES

Berkov, S., Osorio, E., Viladomat, F. & Bastida, J. Chapter Two-Chemodiversity, chemotaxonomy and chemoccology of Amaryllidaceae alkaloids. inThe Alkaloids: Chemistry and Biology (ed. Knölker, H.-J.) vol. 83 113-185 (Academic Press, 2020).
Evidente, A.et al.Biological Evaluation of Structurally Diverse Amaryllidaceae Alkaloids and their Synthetic Derivatives: Discovery of Novel Leads for Anticancer Drug Design.Planta Med. 75, 501-507 (2009).
Cahlíková, L.et al. The Amaryllidaceae alkaloids hacmanthamine, haemanthidine and their semisynthetic derivatives as potential drugs. Phytochem. Rev. 20, 303-323 (2021).
Roy, M.et al.Lycorine: A prospective natural lead for anticancer drug discovery.Biomed. Pharmacother. 107, 615-624 (2018).
Bhattacharya, S., Maclicke, A. & Montag, D. Nasal Application of the Galantamine Pro-drug Memogain Slows Down Plaque Deposition and Ameliorates Behavior in 5×Familial Alzheimer's Disease Mice.J. Alzheimers Dis. 46, 123-136 (2015).
Mucke, H. A. The case of galantamine: repurposing and late blooming of a cholinergic drug.Future Sci. OA1, (2015).
Akram, M. N., Verpoorte, R. & Pomahačová, B. Effect of bulb age on alkaloid contents of narcissus pseudonarcissus bulbs.South Afr. J. Bot. 136, 182-189 (2021).
Marco-Contelles, J., do Carmo Carreiras, M., Rodríguez, C., Villarroya, M. & García, A. G. Synthesis and Pharmacology of Galantamine. Chem. Rev. 106, 116-133 (2006).
Fraser, M. D., Vallin, H. E., Davies, J. R. T., Rowlands, G. E. & Chang, X. Integrating Narcissus-derived galanthamine production into traditional upland farming systems.Sci. Rep. 11, 1389 (2021).
Effect of Fertilizers on Galanthamine and Metabolite Profiles in Narcissus Bulbs by 1H NMR| Journal of Agricultural and Food Chemistry. https://pubs-acs-org.czproxy.lib.utexas.cdu/doi/10.1021/jf104422m.
Thodey, K., Galanic, S. & Smolke, C. D. A microbial biomanufacturing platform for natural and semisynthetic opioids.Nat. Chem. Biol. 10, 837-844 (2014).
Payne, J. T., Valentic, T. R. & Smolke, C. D. Complete biosynthesis of the bisbenzylisoquinoline alkaloids guattegaumerine and berbamunine in yeast.Proc. Natl. Acad. Sci. 118, e2112520118 (2021).
Srinivasan, P. & Smolke, C. D. Biosynthesis of medicinal tropane alkaloids in yeast.Nature585, 614-619 (2020).
Zhang, J.et al.A microbial supply chain for production of the anti-cancer drug vinblastine.Nature609, 341-347 (2022).
Kilgore, M. B. & Kutchan, T. M. The Amaryllidaceae alkaloids: biosynthesis and methods for enzyme discovery.Phytochem. Rev. 15, 317-337 (2016).
Ehrenworth, A. M. & Peralta-Yahya, P. Accelerating the semisynthesis of alkaloid-based drugs through metabolic engineering.Nat. Chem. Biol. 13, 249-258 (2017).
d′Oelsnitz, S.et al. Using fungible biosensors to evolve improved alkaloid biosyntheses. Nat. Chem. Biol. 18, 981-989 (2022).
Schendziclorz, G.et al.Taking Control over Control: Use of Product Sensing in Single Cells to Remove Flux Control at Key Enzymes in Biosynthesis Pathways.ACS Synth. Biol. 3, 21-29 (2014).
Zhang, J.et al.Combining mechanistic and machine learning models for predictive engineering and optimization of tryptophan metabolism.Nat. Commun. 11, 4880 (2020).
Tang, S.-Y.et al.Screening for Enhanced Triacetic Acid Lactone Production by Recombinant Escherichia coli Expressing a Designed Triacetic Acid Lactone Reporter.J. Am. Chem. Soc. 135, 10099-10103 (2013).
Lu, H.et al. Machine learning-aided engineering of hydrolases for PET depolymerization.Nature604, 662-667 (2022).
Hic, B. L. & Yang, K. K. Adaptive machine learning for protein engineering.Curr. Opin. Struct. Biol. 72, 145-152 (2022).
Greenhalgh, J. C., Fahlberg, S. A., Pfleger, B. F. & Romero, P. A. Machine learning-guided acyl-ACP reductase engineering for improved in vivo fatty alcohol production.Nat. Commun. 12, 5825 (2021).
Wu, Z., Kan, S. B. J., Lewis, R. D., Wittmann, B. J. & Arnold, F. H. Machine learning-assisted directed protein evolution with combinatorial libraries.Proc. Natl. Acad. Sci. U.S.A. 116, 8852-8858 (2019).
McNutt, A. T.et al.GNINA 1.0: molecular docking with deep learning.J. Cheminformatics13, 43 (2021).
Jumper, J.et al. Highly accurate protein structure prediction with AlphaFold.Nature596, 583-589 (2021).
Kilgore, M. B.et al.Cloning and Characterization of a Norbelladine 4′-O-Methyltransferase Involved in the Biosynthesis of the Alzheimer's Drug Galanthamine in Narcissus sp. aff. pseudonarcissus.PLOS ONE9, e 103223 (2014).
Cravens, A., Payne, J. & Smolke, C. D. Synthetic biology strategies for microbial biosynthesis of plant natural products.Nat. Commun. 10, 2142 (2019).
Shroff, R.et al.Discovery of Novel Gain-of-Function Mutations Guided by Structure-Based Deep Learning.ACS Synth. Biol. 9, 2927-2935 (2020).
Paik, I.et al.Improved Bst DNA Polymerase Variants Derived via a Machine Learning Approach.Biochemistry62, 410-418 (2023).
Kulikova, A. V., Diaz, D. J., Loy, J. M., Ellington, A. D. & Wilke, C. O. Learning the local landscape of protein structures with convolutional neural networks.J. Biol. Phys. 47, 435-454 (2021).
He, K., Zhang, X., Ren, S. & Sun, J. Deep Residual Learning for Image Recognition. Preprint at https://doi.org/10.48550/arXiv.1512.03385 (2015).
He, K., Zhang, X., Ren, S. & Sun, J. Identity Mappings in Deep Residual Networks. Preprint at https://doi.org/10.48550/arXiv.1603.05027 (2016).
Stourac, J.et al.FireProtDB: database of manually curated protein stability data.Nucleic Acids Res. 49, D319-D324 (2021).
Newton, R. J., Hay, F. R. & Ellis, R. H. Temporal patterns of seed germination in early spring-flowering temperate woodland geophytes are modified by warming.Ann. Bot. 125, 1013-1023 (2020).
Ferrer, J.-L., Zubieta, C., Dixon, R. A. & Nocl, J. P. Crystal Structures of Alfalfa Caffeoyl Coenzyme A 3-O-Methyltransferase.Plant Physiol. 137, 1009-1017 (2005).
Jin, J.-Q.et al.Characterization of two O-methyltransferases involved in the biosynthesis of O-methylated catechins in tea plant.Nat. Commun. 14, 5075 (2023).
Baek, M.et al.Accurate prediction of protein structures and interactions using a three-track neural network.Science373, 871-876 (2021).
Lin, Z.et al.Evolutionary-scale prediction of atomic-level protein structure with a language model.Science379, 1123-1130 (2023).
Wu, R.et al.High-resolution de novo structure prediction from primary sequence. 2022.07.21.500999 Preprint at https://doi.org/10.1101/2022.07.21.500999 (2022).
Watson, J. L.et al. De novo design of protein structure and function with RFdiffusion.Nature620, 1089-1100 (2023).
Eguchi, R. R., Choc, C. A. & Huang, P.-S. Ig-VAE: Generative modeling of protein structure by direct 3D coordinate generation.PLOS Comput. Biol. 18, e1010271 (2022).
Hekkelman, M. L., de Vries, I., Joosten, R. P. & Perrakis, A. AlphaFill: enriching AlphaFold models with ligands and cofactors. Nat. Methods20, 205-213 (2023).
Zhang, Y., Ma, L., Su, P., Huang, L. & Gao, W. Cytochrome P450s in plant terpenoid biosynthesis: discovery, characterization and metabolic engineering. Crit. Rev. Biotechnol. 43, 1-21 (2023).
Noda, S.et al. Evaluation of Brachypodium distachyon L-Tyrosine Decarboxylase Using L-Tyrosine Over-Producing Saccharomyces cerevisiae. PLOS ONE10, c0125488 (2015).
Curran, K. A., Leavitt, J. M., Karim, A. S. & Alper, H. S. Metabolic engineering of muconic acid production in Saccharomyces cerevisiae. Metab. Eng. 15, 55-66 (2013).
Abatemarco, J.et al. RNA-aptamers-in-droplets (RAPID) high-throughput screening for secretory phenotypes. Nat. Commun. 8, 332 (2017).
Kilgore, M. B., Holland, C. K., Jez, J. M. & Kutchan, T. M. Identification of a Noroxomaritidine Reductase with Amaryllidaceae Alkaloid Biosynthesis Related Activities *. J. Biol. Chem. 291, 16740-16752 (2016).
Singh, A.et al. Cloning and characterization of norbelladine synthase catalyzing the first committed reaction in Amaryllidaceae alkaloid biosynthesis. BMC Plant Biol. 18, 338 (2018).
Tousignant, L.et al.Transcriptome analysis of Leucojum aestivum and identification of genes involved in norbelladine biosynthesis.Planta255, 30 (2022).
Kilgore, M. B., Augustin, M. M., May, G. D., Crow, J. A. & Kutchan, T. M. CYP96T1 of Narcissus sp. aff. pseudonarcissus Catalyzes Formation of the Para-Para′ C-C Phenol Couple in the Amaryllidaceae Alkaloids. Front. Plant Sci. 7, (2016).
Mehta, N., Meng, Y., Zare, R., Kamenetsky-Goldstein, R. & Sattely, E. A developmental gradient reveals biosynthetic pathways to eukaryotic toxins in monocot geophytes. 2023.05.12.540595 Preprint at https://doi.org/10.1101/2023.05.12.540595 (2023).
Raček, T. et al. Atomic Charge Calculator II: web-based tool for the calculation of partial atomic charges. Nucleic Acids Res. 48, W591-W596 (2020).
Mitternacht, S. FreeSASA: An open source C library for solvent accessible surface area calculations. Preprint at https://doi.org/10.12688/f1000research. 7931.1 (2016).
Wojdyr, M. GEMMI: A library for structural biology. J. Open Source Softw. 7, 4200 (2022).
Johnson, K. A., Simpson, Z. B. & Blom, T. Global Kinetic Explorer: A new computer program for dynamic simulation and fitting of kinetic data. Anal. Biochem. 387, 20-29 (2009).
Johnson, K. A., Simpson, Z. B. & Blom, T. FitSpace Explorer: An algorithm to evaluate multidimensional parameter space in fitting kinetic data. Anal. Biochem. 387, 30-41 (2009).
Park, J. B. Synthesis and characterization of norbelladine, a precursor of Amaryllidaceae alkaloid, as an anti-inflammatory/anti-COX compound. Bioorganic & Medicinal Chemistry Letters 24, 5381-5384 (2014).
Stourac, J., Dubrava, J., Musil, M., Horackova, J., Damborsky, J., Mazurenko, S., Bednar, D., 2020: FireProtDB: Database of Manually Curated Protein Stability Data. Nucleic Acids Research 49: D319-D324.
Mehta N, Meng Y, Zare R, Kamenetsky-Goldstein R, Sattely E. A developmental gradient reveals biosynthetic pathways to eukaryotic toxins in monocot geophytes. bioRxiv [Preprint]. 2023 May 12:2023.05.12.540595.

1. A non-naturally occurring methyltransferase, wherein said methyltransferase can methylate norbelladine to form 4-O'Methylnorbelladine.

2. The methyltransferase of claim 1, wherein when the non-naturally occurring methyltransferase methylates norbelladine to form 4-O'Methylnorbelladine, less 3-O'Methylnorbelladine is formed compared to a native norbelladine methyltransferase.

3. The methyltransferase of claim 2, wherein at least 2-fold less 3-O'Methylnorbelladine is formed.

4. The methyltransferase of claim 1, wherein the methyltransferase is at least 5% more active than a non-native methyltransferase from which it is derived, as represented by SEQ ID NO 3.

5. The methyltransferase of claim 1, wherein the methyltransferase is represented as a protein with 90% or more identity to any one of SEQ ID NO: 4-8, 17, or 18, wherein for SEQ ID NO: 4, position 53M does not vary; for SEQ ID NO: 5, position 159E does not vary; for SEQ ID NO: 6, position 203E does not vary; for SEQ ID NO: 7, position 17K does not vary; for SEQ ID NO: 8, neither position 36P nor position 40E vary, for SEQ ID NO: 17, position 17R does not vary, and for SEQ ID NO: 18, none of positions 36P, 40E, not 53M vary.

6. The methyltransferase of claim 1, wherein the methyltransferase has 95% or more identity to any one of SEQ ID NO: 4-8, 17, or 18, wherein for SEQ ID NO: 4, position 53M does not vary; for SEQ ID NO: 5, position 159E does not vary; for SEQ ID NO: 6, position 203E does not vary; for SEQ ID NO: 7, position 17K does not vary; for SEQ ID NO: 8, neither position 36P nor position 40E vary, for SEQ ID NO: 17, position 17R does not vary, and for SEQ ID NO: 18, none of positions 36P, 40E, not 53M vary. The methyltransferase of claim 6, wherein the methyltransferase comprises any of SEQ ID NOS: 4-8, 17, or 18.

7. A nucleic acid encoding the methyltransferase of claim 1.

8. A host cell comprising the nucleic acid of claim 7.

9. The host cell of claim 8, wherein the cell further comprises a second nucleic acid encoding a protein from a different organism than the host cell.

10. The host cell of claim 9, wherein the host cell also comprises a third nucleic acid encoding a protein from a different organism than the host cell.

11. The host cell of claim 9, wherein the host cell comprises at least one additional nucleic acid which encodes a non-naturally occurring protein.

12. The host cell of claim 9, wherein the host cell encodes nucleic acids encoding two or more components of an amaryllidaceae alkaloid pathway.

13. The host cell of claim 12, wherein the amaryllidaceae alkaloid pathway produces galantamine, lycorine, crinine, or haemanthamine.

14. The methyltransferase of claim 1, wherein the methyltransferase is in a cell-free environment.

15. A method of preparing an amaryllidaceae alkaloid, wherein the amaryllidaceae alkaloid composition requires methylation of norbelladine to form 4-O'Methylnorbelladine, the method comprising:
a. culturing a host cell under suitable conditions, wherein the host cell comprises nucleic acid encoding a non-naturally occurring methyltransferase;
b. exposing the methyltransferase to norbelladine; and
c. allowing the methyltransferase to methylate norbelladine, thereby producing a methylated composition of interest.

16-23. (canceled)

24. A biosensor for detecting 4-O'Methylnorbelladine, wherein the biosensor comprises an engineered substrate-promiscuous regulator, wherein said substrate-promiscuous regulator has been engineered to interact more efficiently with 4-O'Methylnorbelladine than does a naturally occurring substrate promiscuous regulator; and further wherein the biosensor is engineered to provide an output signal, wherein said output signal is generated when the biosensor interacts with 4-O'Methylnorbelladine.

25. The biosensor of claim 24, wherein the engineered biosensor is derived from RamR of Salmonella typhimurium.

26. The biosensor of claim 25, wherein the biosensor comprises at least one substitution of K63T and/or L66M compared to native RamR (as represented by SEQ ID NO: 3).

27-46. (canceled)

47. A kit comprising a 4-O'Methylnorbelladine biosensor comprising an engineered substrate-promiscuous regulator, wherein said substrate-promiscuous regulator has been engineered to interact more efficiently with 4-O'Methylnorbelladine than does the naturally occurring substrate promiscuous regulator.

48-60. (canceled)

61. A method comprising:
a. First generating a 3D molecular structural model of a protein with a machine learning structure prediction tools (such as AlphaFold, RosettaFold, OmegaFold, ESMFold, OpenFold);
b. Docking the 1 or more relevant ligands, including cofactors, substrates, and products to the computationally generated 3D protein structure; and
c. using the generated protein-ligand(s) complex as input to a trained AI classifier to generate one or more mutation predictions of residues within the first and second contact shell of the ligands.

62-66. (canceled)
